(19) United States
(12) Patent Application Publication
(10) Pub. No.: US 2025/0275965 A1
(43) Pub. Date: Sep. 4, 2025

(54) COMPOUNDS AS CASEIN KINASE INHIBITORS

(71) Applicant: GRITSCIENCE BIOPHARMACEUTICALS CO.,LTD., Beijing (CN)

(72) Inventors: Enxing ZHOU, Beijing (CN); Yuan LIU, Beijing (CN); Hanping WANG, Beijing (CN); Guanglong WU, Beijing (CN); Wei DENG, Beijing (CN); Guosheng WU, Beijing (CN); Yulai ZHANG, Beijing (CN); Shengquan LIU, Beijing (CN)

(21) Appl. No.: 18/265,673

(22) PCT Filed: Dec. 14, 2021
(86) PCT No.: PCT/CN2021/137685

§ 371 Date: Jun. 6, 2023

(30) Foreign Application Priority Data
Dec. 15, 2020 (WO) PCT/CN2020/136537

(51) Int. Cl. A61K 31/5025; A61K 31/4365; A61K 31/519; C07D 487/04; C07D 495/04; C07D 519/00

(52) U.S. Cl. CPC A61K 31/5025; A61K 31/4365; A61K 31/519; C07D 487/04; C07D 495/04; C07D 519/00

(57) ABSTRACT

Provided herein are casein kinase inhibitors, or pharmaceutically acceptable salts thereof. Corresponding compositions, methods of treatment, and intermediates are also provided.

### BACKGROUND OF THE INVENTION

The circadian clock links our daily cycles of sleep and activity to the external environment. Deregulation of the clock is implicated in a number of human disorders, including depression, seasonal affective disorder, and metabolic disorders. For example, the circadian clock may regulate multiple downstream rhythms, such as those in sleep and awakening, body temperature, and hormone secretion (Ko and Takahashi, Hum Mol Gen 15: R271-R277.). Furthermore, diseases such as depression, seasonal affective disorder, and metabolic disorders, may have a circadian origin (Barnard and Nolan, PLoS Genet. 2008 May; 4(5): e1000040.).

Casein kinase (CK) is closely related Ser-Thr protein kinases that serve as key clock regulators that dramatically alter the circadian period. There is a continuing need for CK inhibitors in the treatment of diseases.

### SUMMARY OF THE INVENTION

The present disclosure provides a series of compounds as potent inhibitors of casein kinase.

In one aspect, the present application provides a compound having the structure of formula (I),

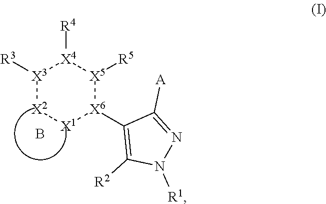

or a pharmaceutically acceptable salt, or prodrug thereof, or a solvate or hydrate of any of the forgoing,
wherein,
each A and B is independently selected from the group consisting of optionally substituted C₆-C₁₄ aryl, and optionally substituted C₂-C₉ heteroaryl;
each dashed line (---) represents a single or double bond, each X¹, X², X³, X⁴, X⁵ and X⁶ is independently selected from the group consisting of C, N, and optionally substituted CH;
R¹ is selected from the group consisting of hydrogen, protium, deuterium, tritium, halogen, cyano, nitro, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl;
R² is selected from the group consisting of hydrogen, protium, deuterium, tritium, halogen, cyano, nitro, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl,
or R¹ and R² combined with the atoms to which they are attached form an optionally substituted ring;
each R³, R⁴ and R⁵ is independently absent or is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl,
or R³ and R⁴ combined with the atoms to which they are attached form an optionally substituted ring,
or R³ and R⁵ combined with the atoms to which they are attached form an optionally substituted ring,
or R⁴ and R⁵ combined with the atoms to which they are attached form an optionally substituted ring.

In some embodiments, wherein, said B is optionally substituted C₂-C₉ heteroaryl.

In some embodiments, wherein, said B is selected from the group consisting of optionally substituted pyrazole, optionally substituted imidazole, optionally substituted thiophene, optionally substituted pyrrole, and optionally substituted triazole.

In some embodiments, wherein, said B is optionally substituted imidazole.

In some embodiments, wherein, said B is substituted with one or more R⁶, each R⁶ is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl.

In some embodiments, wherein, each R⁶ is independently selected from the group consisting of hydrogen, halogen, ═O, optionally substituted (C₁-C₆)alkyl, optionally substituted (C₁-C₆)acyl, and optionally substituted amino.

In some embodiments, wherein, each R⁶ is independently selected from the group consisting of optionally substituted methyl, optionally substituted ethyl and optionally substituted isopropyl.

In some embodiments, wherein, said R⁶ is substituted with one or more R⁷, each R⁷ is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl.

In some embodiments, wherein, said R⁷ is independently selected from the group consisting of hydrogen, optionally substituted (C₁-C₆)alkyl, optionally substituted (C₁-C₆)acyl, optionally substituted amino, and optionally substituted hydroxy.

In some embodiments, wherein, said R⁷ is substituted with one or more R⁸, each R⁸ is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl.

In some embodiments, wherein, said R⁸ is independently selected from the group consisting of hydrogen, optionally substituted (C₁-C₆)alkyl, optionally substituted (C₃-C₁₀)carbocycle, and optionally substituted (C₁-C₆)acyl.

In some embodiments, wherein, said R⁸ is independently selected from the group consisting of optionally substituted methyl and optionally substituted cyclopropyl.

In some embodiments, wherein, said R⁸ is substituted with one or more R⁹, each R⁹ is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl.

In some embodiments, wherein, said R⁹ is independently selected from the group consisting of hydrogen, halogen and optionally substituted (C₁-C₆)alkyl.

In some embodiments, wherein, each X¹ and X² is independently selected from the group consisting of C and N.

In some embodiments, wherein, said X¹ is C and said X² is N.

In some embodiments, wherein the compound has the structure selected from the group consisting of

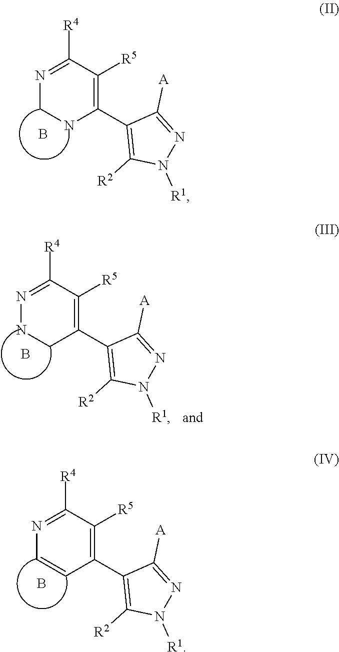

In some embodiments, wherein, each R⁴ and R⁵ is independently selected from the group consisting of hydrogen, protium, deuterium, tritium, halogen, cyano, nitro, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl.

In some embodiments, wherein, each R⁴ and R⁵ is independently selected from the group consisting of hydrogen and halogen.

In some embodiments, wherein, said R⁴ is hydrogen and said R⁵ is hydrogen.

In some embodiments, wherein, said R¹ is optionally substituted (C₁-C₆)alkyl.

In some embodiments, wherein, said R¹ is optionally substituted methyl.

In some embodiments, wherein, said R² is hydrogen.

In some embodiments, wherein, R¹ and R² combined with the atoms to which they are attached form an optionally substituted ring C, said ring C is selected from the group consisting of optionally substituted (C₃-C₁₀) carbocycle, optionally substituted (C₂-C₉) heterocycle, optionally substituted (C₆-C₁₀) aryl, and optionally substituted (C₁-C₉) heteroaryl.

In some embodiments, wherein, said ring C is optionally substituted (C₂-C₉) heterocycle.

In some embodiments, wherein, said ring C is optionally substituted piperazine.

In some embodiments, wherein, said ring C is substituted with one or more R¹⁰, each R¹⁰ is independently absent or is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl.

In some embodiments, wherein, said R¹⁰ is independently selected from the group consisting of optionally substituted (C₁-C₆)acyl and optionally substituted (C₁-C₆)alkyl.

In some embodiments, wherein, said A is optionally substituted C₆-C₁₄ aryl.

In some embodiments, wherein, said A is optionally substituted phenyl.

In some embodiments, wherein, said A is substituted with one or more R¹¹, each R¹¹ is independently absent or is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl.

In some embodiments, wherein, said R¹¹ is halogen.

In some embodiments, wherein, said R¹¹ is F.

In another aspect, the present application provides a compound or a pharmaceutically acceptable salt, prodrug, or metabolite thereof, or a solvate or hydrate of any of the foregoing, wherein, said compound is selected from the group consisting of:

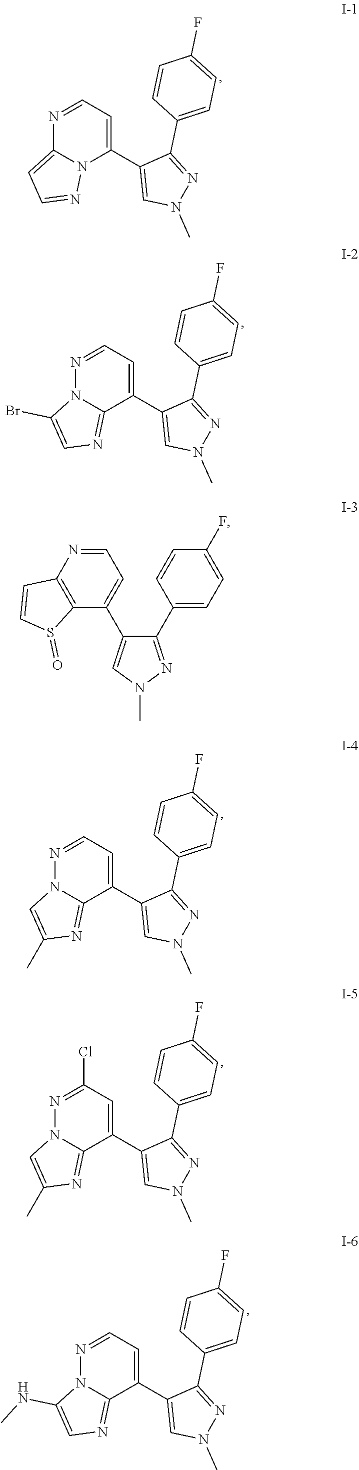

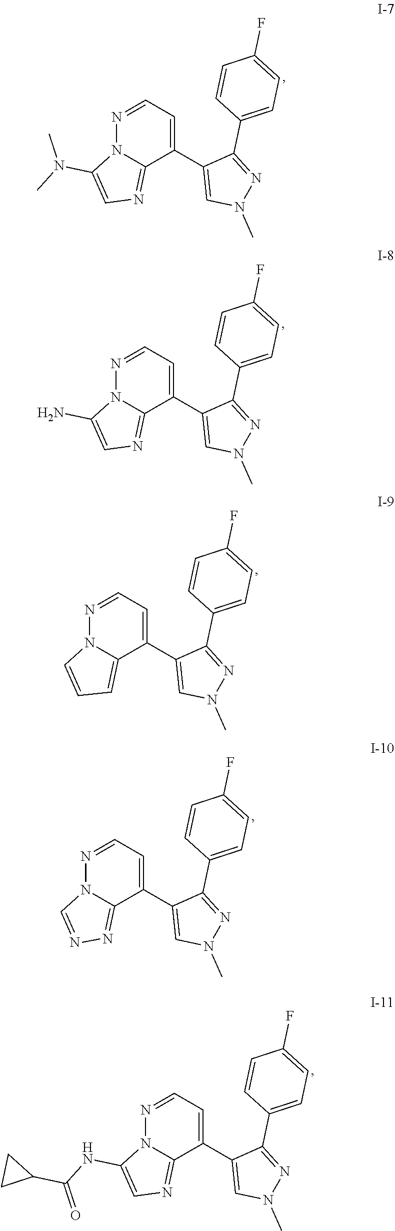

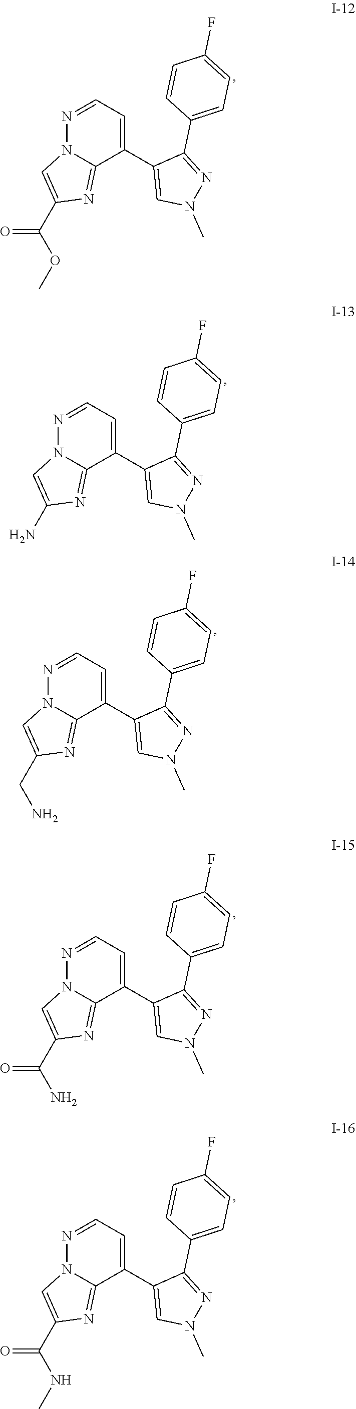

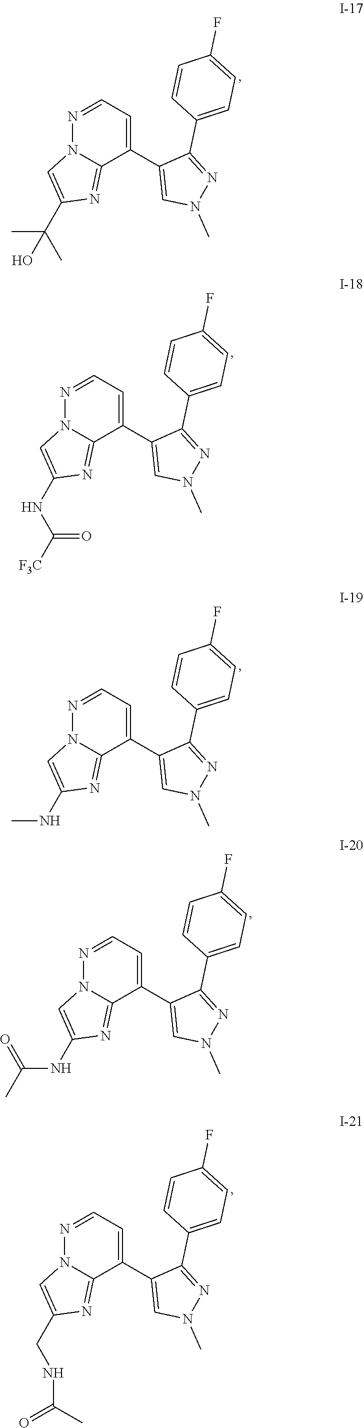

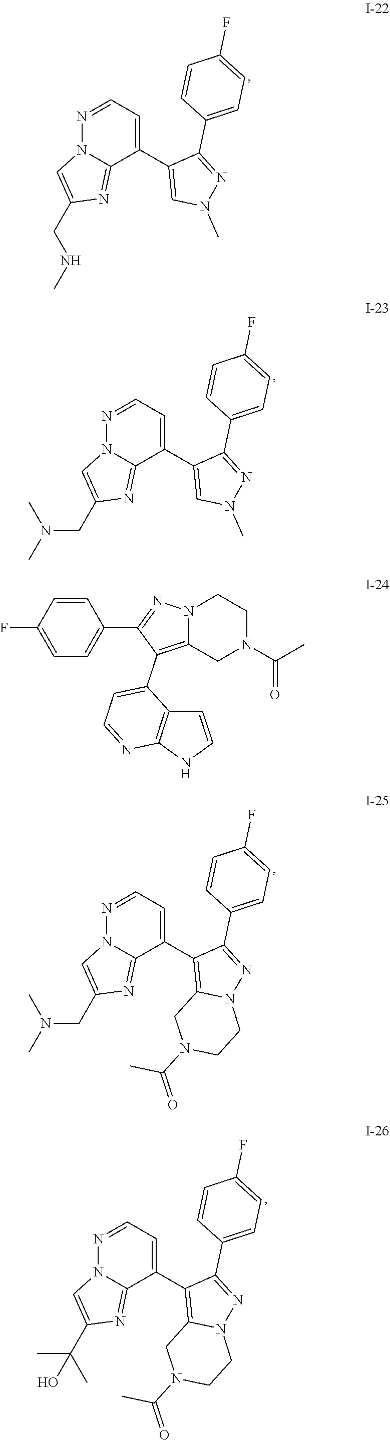

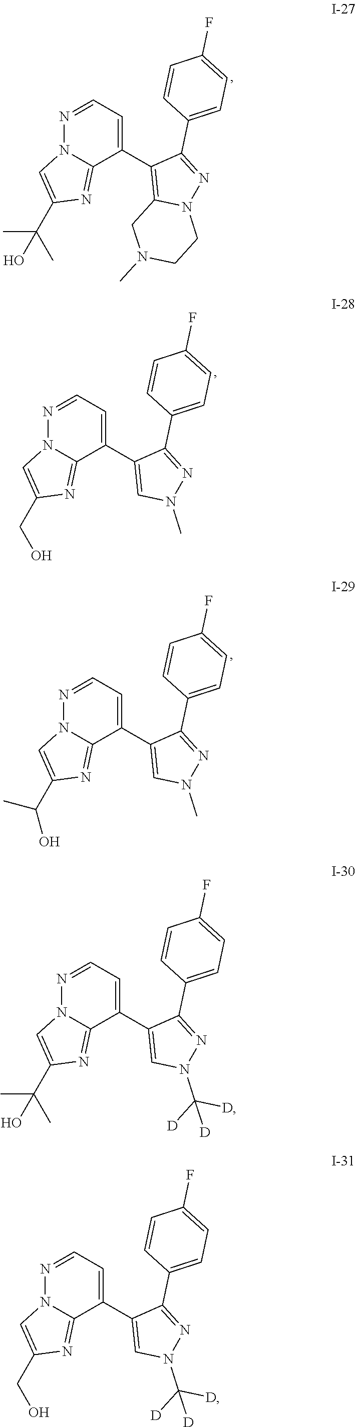

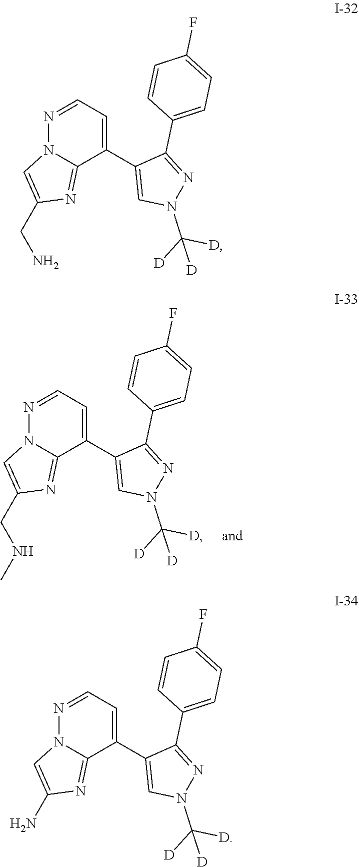

In another aspect, the present application provides a composition comprising a compound of any one of formula (I), or a pharmaceutically acceptable salt, prodrug, or metabolite thereof, or a solvate or hydrate of any of the foregoing, and optionally a pharmaceutically acceptable carrier.

In another aspect, the present application provides a method for inhibiting casein kinase (CK) activity, said method comprising administering to a subject in need thereof an effective amount of the compound of any of formula (I), or a pharmaceutically acceptable salt, prodrug, or metabolite thereof, or a solvate or hydrate of any of the foregoing.

In some embodiments, wherein said casein kinase (CK) is selected from the group consisting of casein kinase I alpha (CK1α), casein kinase I delta (CK1δ) and casein kinase I epsilon (CK1ε).

In some embodiments, wherein said method is selected from the group consisting of an in vitro method, an ex vivo method, and an in vivo method.

In another aspect, the present application provides a method for preventing and/or treating a disease or disorder, said method comprising administering to a subject in need thereof an effective amount of the compound of any of formula (I), or a pharmaceutically acceptable salt, prodrug, or metabolite thereof, or a solvate or hydrate of any of the foregoing.

In some embodiments, wherein said disease or disorder is selected from the group consisting of neurological disease and psychiatric disease.

In some embodiments, wherein said disease or disorder is selected from the group consisting of mood disorder, sleep disorder, and circadian disorder.

In some embodiments, wherein said disease or disorder is selected from the group consisting of depressive disorder and bipolar disorder.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIG. 1 to FIG. 29 illustrate the synthetic schemes of compound I-1 to compound I-29.

FIG. 30a and FIG. 30b illustrate the synthetic scheme of compound I-30.

FIG. 31 to FIG. 34 illustrate the synthetic schemes of compound I-31 to compound I-34.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

As used herein, the term “alkyl”, either alone or within other terms, generally refers to a linear or branched-chain saturated hydrocarbyl substituent (i.e., a substituent obtained from a hydrocarbon by removal of a hydrogen) containing from one to twenty carbon atoms; for example, from one to twelve carbon atoms; in another example, from one to ten carbon atoms; in another embodiment, from one to six carbon atoms; and in another embodiment, from one to four carbon atoms (such as 1, 2, 3 or more carbon atoms). Examples of such substituents may include e.g., methyl, ethyl, propyl (including n-propyl and isopropyl), butyl (including n-butyl, isobutyl, sec-butyl and terf-butyl), pentyl, isoamyl, hexyl and the like. In some instances, the number of carbon atoms in a hydrocarbyl substituent (i.e., alkyl, alkenyl, cycloalkyl, aryl, etc.) may be indicated by the prefix “Cₐ-C_{b}” wherein a is the minimum and b is the maximum number of carbon atoms in the substituent. Thus, for example, “C₁-C₆ alkyl” may refer to an alkyl substituent containing from 1 to 6 carbon atoms. The “alkyl” groups may be optionally substituted with one or more substitutions.

As used herein, the term “alkenyl”, either alone or within other terms, generally refers to a linear or branched-carbon radicals having at least one carbon-carbon double bond. The term “alkenyl” may contain conjugated and non-conjugated carbon-carbon double bonds or combinations thereof. An alkenyl group, for example and without being limited thereto, may contain two to about twenty carbon atoms or, in a particular embodiment, two to about twelve carbon atoms. In embodiments, alkenyl groups may contain two to about four carbon atoms (such as 2, 3 or more carbon atoms). Examples of alkenyl groups include, but are not limited thereto, ethenyl, propenyl, allyl, propenyl, butenyl and 4-methylbutenyl. The terms “alkenyl” contain groups having “cis” and “trans” orientations, or alternatively, “E” and “Z” orientations. In some instances, the number of carbon atoms may be indicated by the prefix “Cₐ-C_{b}” wherein a is the minimum and b is the maximum number of carbon atoms in the substituent. The “alkenyl” groups may be optionally substituted with one or more substitutions.

As used herein, the term “alkynyl”, either alone or within other terms, generally refers to linear or branched carbon radicals having at least one carbon-carbon triple bond. The term “alkynyl” may contain conjugated and non-conjugated carbon-carbon triple bonds or combinations thereof. Alkynyl group, for example and without being limited thereto, may contain two to about twenty carbon atoms or, in a particular embodiment, two to about twelve carbon atoms. In embodiments, alkynyl groups may contain two to about ten carbon atoms. Some examples may be alkynyl having two to about four carbon atoms (such as 2, 3 or more carbon atoms). In some instances, the number of carbon atoms may be indicated by the prefix “Cₐ-C_{b}” wherein a is the minimum and b is the maximum number of carbon atoms in the substituent. Examples of such groups include propargyl, butynyl, and the like. The “alkynyl” groups may be optionally substituted with one or more substitutions.

As used herein, the term “amino”, either alone or within other terms, generally refers to formula —NH₂ group. The “amino” groups may be optionally substituted with one or more substitutions.

As used herein, the term “carbocycle”, either alone or within other terms, generally refers to a saturated or unsaturated non-aromatic monocyclic, bicyclic, or polycyclic ring system having from 3 to 14 ring atoms (and all combinations and subcombinations of ranges and specific numbers of carbon atoms therein) wherein all of the ring atoms are carbon atoms. Monocyclic carbocycles may have 3 to 6 ring atoms, or 5 to 6 ring atoms. Bicyclic carbocycles may have 7 to 12 ring atoms, e.g., arranged as a bicyclo[4,5], [5,5], [5,6] or [6,6] system, or 9 or 10 ring atoms arranged as a bicyclo[5,6] or [6,6] system. The term “carbocycle” may contain, for example, a monocyclic carbocycle ring fused to an aryl ring (e.g., a monocyclic carbocycle ring fused to a benzene ring). Carbocyles may have 3 to 8 carbon ring atoms. In some instances, the number of carbon atoms may be indicated by the prefix “Cₐ C_{b}” wherein a is the minimum and b is the maximum number of carbon atoms in the substituent. The “carbocycle” groups may be optionally substituted with one or more substitutions.

As used herein, the term “heterocycle”, either alone or within other terms, generally refers to a monocyclic, bicyclic, or polycyclic ring system having from 3 to 14 ring atoms (also referred to as ring members) wherein at least one ring atom in at least one ring may be a heteroatom selected from N, O, P, or S (and all combinations and subcombinations of ranges and specific numbers of carbon atoms and heteroatoms therein). The heterocycle may have from 1 to 4 ring heteroatoms independently selected from N, O, P, or S. One or more N, C, or S atoms in a heterocycle may be oxidized. A monocylic heterocycle may have 3 to 7 ring members (e.g., 2 to 6 carbon atoms and 1 to 3 heteroatoms independently selected from N, O, P, or S), and a bicyclic heterocycle may have 5 to 10 ring members (e.g., 4 to 9 carbon atoms and 1 to 3 heteroatoms independently selected from N, O, P, or S). The heterocycle that contains the heteroatom may be non-aromatic. Unless otherwise noted, the heterocycle is attached to its pendant group at any heteroatom or carbon atom that results in a stable structure. In some instances, the number of carbon atoms may be indicated by the prefix “Cₐ-C_{b}” wherein a is the minimum and b is the maximum number of carbon atoms in the substituent. The “heterocycle” groups may be optionally substituted with one or more substitutions.

As used herein, the term “aryl”, either alone or within other terms, generally refers to an aromatic substituent containing one ring or two or three fused rings, The aryl substituent may have six to eighteen carbon atoms. As an example, the aryl substituent may have six to fourteen carbon atoms. The term “aryl” may refer to substituents such as phenyl, naphthyl and anthracenyl. The term “aryl” may also contain substituents such as phenyl, naphthyl and anthracenyl that are fused to a C₄-C₁₀ carbocyclic ring, such as a C₅ or a C₆ carbocyclic ring, or to a 4- to 10-membered heterocyclic ring, wherein a group having such a fused aryl group as a substituent is bound to an aromatic carbon of the aryl group. When such a fused aryl group is substituted with one more substituent, the one or more substituents, unless otherwise specified, may be each bound to an aromatic carbon of the fused aryl group. The fused C₄-C₁₀ carbocyclic or 4- to 10-membered heterocyclic ring may optionally be optionally substituted. Examples of aryl groups may include accordingly phenyl, naphthalenyl, tetrahydronaphthalenyl (also known as “tetralinyl”), indenyl, isoindenyl, indanyl, anthracenyl, phenanthrenyl, benzonaphthenyl (also known as “phenalenyl”), and fluorenyl. In some instances, the number of carbon atoms may be indicated by the prefix “Cₐ-C_{b}” wherein a is the minimum and b is the maximum number of carbon atoms in the substituent. The “aryl” groups may be optionally substituted with one or more substitutions.

As used herein, the term “heteroaryl”, either alone or within other terms, generally refers to an aromatic ring structure containing from 5 to 14 ring atoms in which at least one of the ring atoms is a heteroatom (for example, oxygen, nitrogen, or sulfur), with the remaining ring atoms being independently selected from the group consisting of carbon, oxygen, nitrogen, and sulfur. A heteroaryl may be a single ring or 2 or 3 fused rings. Examples of heteroaryl substituents may include but not limited to: 6-membered ring substituents such as pyridyl, pyrazyl, pyrimidinyl, and pyridazinyl; 5-membered ring substituents such as triazolyl, imidazolyl, furanyl, thiophenyl, pyrazolyl, oxazolyl, isoxazolyl, thiazolyl, 1,2,3-, 1, 2, 4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6-membered fused ring substituents such as quinolinyl, isoquinolinyl, cinnolinyl, quinazolinyl, and 1,4-benzoxazinyl. In a group that has a heteroaryl substituent, the ring atom of the heteroaryl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heteroaryl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom. In some instances, the number of carbon atoms may be indicated by the prefix “Cₐ-C_{b}” wherein a is the minimum and b is the maximum number of carbon atoms in the substituent. The “heteroaryl” groups may be optionally substituted with one or more substitutions.

As used herein, the term “halogen”, either alone or within other terms, generally refers to fluorine (which may be depicted as —F), chlorine (which may be depicted as —Cl), bromine (which may be depicted as —Br), or iodine (which may be depicted as —I). In one embodiment, the halogen may be chlorine. In another embodiment, the halogen may be fluorine. In another embodiment, the halogen may be bromine.

As used herein, the term “cyano”, either alone or within other terms, generally refers to formula —CN group.

As used herein, the term “nitro”, either alone or within other terms, generally refers to formula —NO₂ group.

As used herein, the term “hydroxy”, either alone or within other terms, generally refers to formula —OH group. The “hydroxy” groups may be optionally substituted with one or more substitutions.

As used herein, the term “phosphorous-containing group”, either alone or within other terms, generally refers to functional group containing on or more phosphorous atoms. The phosphorous-containing group may refer to —O—P—(OH)₂, —O—PH—(OH), —O—PH₂, —P—(OH)₂, —PH—(OH), —PH₄, —PH₂═CH₂, —CH═PH₃, —O—P(═O)₂, —O—P(═O)—(OH)₂, —O—PH(═O)—OH, —P(═O)—(OH)₂, —O—PH₂(═O), —PH(═O)—OH, —PH₂(═O), —O—P(═O)(OH)—P(═O)(OH)₂, —O—P(═O)(OH)—O—P(═O)(OH)₂, —PH—PH₂, or —P═PH. The “phosphorous-containing group” may be optionally substituted with one or more substitutions.

As used herein, the term “silicon-containing group”, either alone or within other terms, generally refers to functional group containing on or more silicon atoms. The silicon-containing group may refer to —SiH₃. The “silicon-containing group” may be optionally substituted with one or more substitutions.

As used herein, the term “thio”, either alone or within other terms, generally refers to formula —SH group. The “thio” groups may be optionally substituted with one or more substitutions.

As used herein, the term “carboxyl”, either alone or within other terms, generally refers to formula —C(═O)OH group. The “carboxyl” groups may be optionally substituted with one or more substitutions.

As used herein, the term “sulfonyl”, either alone or within other terms, generally refers to formula —S(═O)₂—H, group. The “sulfonyl” groups may be optionally substituted with one or more substitutions.

As used herein, the term “sulfinyl”, either alone or within other terms, generally refers to formula —S(═O)—H group. The “sulfinyl” groups may be optionally substituted with one or more substitutions.

As used herein, the term “acyl”, either alone or within other terms, generally refers to a carboxylic acid ester of the formula —C(O)R in which the non-carbonyl moiety of the ester group (i.e., R) may be selected from straight, branched, or cyclic alkyl. The term acyl may include but not limited to acetyl, propionyl, butyryl and pentanoyl. In some instances, the number of carbon atoms may be indicated by the prefix “Cₐ-C_{b}” wherein a is the minimum and b is the maximum number of carbon atoms in the substituent. The “acyl” groups may be optionally substituted with one or more substitutions.

As used herein, the term “thioacyl”, either alone or within other terms, generally refers to the formula —C(S)R in which the moiety of the ester group (i.e., R) may be selected from straight, branched, or cyclic alkyl. In some instances, the number of carbon atoms may be indicated by the prefix “Cₐ-C_{b}” wherein a is the minimum and b is the maximum number of carbon atoms in the substituent. The “thioacyl” groups may be optionally substituted with one or more substitutions.

As used herein, the term “ring”, either alone or within other terms, generally refers to any covalently closed structure. Rings may include, for example, carbocycles, heterocycles, aryls and heteroaryls. Rings may be monocyclic or polycyclic. The “ring” groups may be optionally substituted with one or more substitutions.

As used herein, the term “treating”, unless otherwise indicated, generally refers to reversing, alleviating the progress of, or preventing the disorder or condition to which such term applies, or one or more symptoms of such disorder or condition. The term “treatment”, as used herein, unless otherwise indicated, generally refers to the act of treating as “treating” is defined immediately above. The term “treating” may also include adjuvant and neo-adjuvant treatment of a subject.

As used herein, the term “preventing” unless otherwise indicated, generally refers to precluding, averting, obviating, forestalling, stopping, or hindering something from happening, especially by advance action. It may be understood that where reduce, inhibit or prevent are used herein, unless specifically indicated otherwise, the use of the other two words may be also expressly disclosed.

As used herein, the term “pharmaceutically acceptable salt” generally refers to a salt that may be pharmaceutically acceptable and that may possess the desired pharmacological activity of the parent compound. Such salts may include: acid addition salts, formed with inorganic acids or formed with organic acids or basic addition salts formed with the conjugate bases of any of the inorganic acids wherein the conjugate bases comprise a cationic component.

As used herein, the term “pharmaceutically acceptable carrier” generally refers to aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles may include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity may be maintained, for example, by the use of coating materials such as lecithin, by the maintenance of the required particle size in the case of dispersions and by the use of surfactants. These compositions may also contain adjuvants such as preservatives, wetting agents, emulsifying agents and dispersing agents. Prevention of the action of microorganisms may be ensured by the inclusion of various antibacterial and antifungal agents such as paraben, chlorobutanol, phenol, sorbic acid and the like. It may also be desirable to include isotonic agents such as sugars, sodium chloride and the like. Prolonged absorption of the injectable pharmaceutical form may be brought about by the inclusion of agents, such as aluminum monostearate and gelatin, which delay absorption. Injectable depot forms may be made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release may be controlled. Depot injectable formulations may be also prepared by entrapping the drug in liposomes or microemulsions which are compatible with body tissues. The injectable formulations may be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers may include sugars such as lactose. Desirably, at least 95% by weight of the particles of the active ingredient may have an effective particle size in the range of 0.01 to 10 micrometers.

As used herein, the term “prodrug” generally refers to a compound that is metabolized, for example hydrolyzed or oxidized, in the host to form the compound of the present invention. Typical examples of prodrugs may include compounds that have biologically labile protecting groups on a functional moiety of the active compound. Prodrugs may include compounds that can be oxidized, reduced, aminated, deaminated, hydroxylated, dehydroxylated, hydrolyzed, dehydrolyzed, alkylated, dealkylated, acylated, dedcylated, phosphorylated, dephosphorylated to produce the active compound.

As used herein, the term “casein kinase” generally refers to a protein having an activity of catalyzing the serine/threonine-selective phosphorylation of proteins. This activity may be referred to as “casein kinase activity”. The Gene ID for gene encoding casein kinase may be 1453 or 1454.

As used herein, the term “subject” generally refers to an animal, which may include, but not limited to, cattle, pigs, sheep, chicken, turkey, buffalo, llama, ostrich, dogs, cats, and humans, and the subject may be a human. It may be contemplated that in the method of treating a subject thereof of the sixth embodiment can be any of the compounds either alone or in combination with another compound of the present invention.

As used herein, the term “effective amount” generally refers to an amount of an agent or a compound being administered which will treat a disease or disorder, or some or all of the symptom. The result can be reduction and/or alleviation of the signs, symptoms, or causes of a disease or disorder, or any other desired alteration of a biological system. For example, an “effective amount” for therapeutic uses is the amount of the composition including a compound as disclosed herein required to provide a clinically significant decrease in a disease or disorder symptoms without undue adverse side effects.

As used herein, the term “administering” generally refers to the compound may be administered by any appropriate route, for example, orally, parenterally, intravenously, intradermally, subcutaneously, or topically, in liquid or solid form.

As used herein, a substituent is “substitutable” or can be “substituted” if it comprises at least one atom that is bonded to one or more hydrogen atoms. If a substituent is described as being “substituted,” hydrogen or a non-hydrogen substituent is in the place of a hydrogen substituent on a atom of the substituent. Thus, for example, a substituted alkyl substituent is an alkyl substituent wherein at least one hydrogen or a non-hydrogen substituent is in the place of a hydrogen substituent on the alkyl substituent. To illustrate, monofluoroalkyl is alkyl substituted with a fluoro substituent, and difluoroalkyl is alkyl substituted with two fluoro substituents. It should be recognized that if there is more than one substitution on a substituent, each substituent may be identical or different (unless otherwise stated).

If substituents are described as being “independently selected” from a group, each substituent may be selected independent of the other(s). Each substituent therefore may be identical to or different from the other substituent(s).

As used herein, the term “optionally substituted” generally refers to a given moiety may consist of only hydrogen substituents through available valencies (unsubstituted) or may further comprise one or more non-hydrogen substituents through available valencies (substituted) that are not otherwise specified by the name of the given moiety. For example, “R^{x} is optionally substituted” or R^{x} is optionally substituted with R^{y}” may mean that R^{x} may be substituted with 0, 1, 2, 3, 4, 5, 6, 7, 8, or 9 R^{y}, for example, Rt may be substituted with 0, 1, 2, 3, 4, or 5 R^{y}, for example, R^{x} may be substituted with 1, 2, or 3 R^{y}, for example, R^{x} may be substituted with one R^{y}, for example, R^{x} may be substituted with 2 R^{y}, for example, R^{x} may be substituted with 3 R^{y}, for example, R^{x} may be substituted with 4 R^{y}, for example, R^{x} may be substituted with 5 R^{y}, for example, R^{x} may be substituted with 6 R^{y}, for example, R^{x} may be substituted with 7 R^{y}, for example, R^{x} may be substituted with 8 R^{y}, for example, R^{x} may be substituted with 9 R^{y}. In general, a non-hydrogen substituent may be any substituent that may be bound to an atom of the given moiety that is specified to be substituted. Examples of substituents include, but are not limited to, hydrogen, protium, deuterium, tritium, halogen, cyano, nitro, ═S, ═O, N₃, trifluoromethyl, hydroxy, phosphorous-containing group, silicon-containing group, thio, amino, carboxyl, sulfonyl, sulfinyl, (C₁-C₆)acyl, (C₁-C₆)thioacyl, (C₁-C₆)alkyl, (C₂-C₆)alkenyl, (C₂-C₆)alkynyl, (C₃-C₁₀)carbocycle, (C₂-C₉)heterocycle, (C₆-C₁₀)aryl, (C₁-C₉)heteroaryl, trifluoromethyl(C₁-C₆)alkyl, cyano(C₁-C₆)alkyl, halo(C₁-C₆)alkyl, nitro(C₁-C₆)alkyl, hydroxy(C₁-C₆)alkyl, (C₁-C₆)alkoxy, (C₁-C₆)alkylthio, thio(C₁-C₆)alkyl, amino(C₁-C₆)alkyl, (C₁-C₆)alkylamino, ((C₁-C₆)alkyl)₂amino, (C₁-C₆)acyl(C₁-C₆)alkyl, (C₁-C₆)alkylsulfonyl, (C₁-C₆)alkylsulfinyl, hydroxysulfonyl, hydroxysulfinyl, (C₃-C₁₀)carbocycle(C₁-C₆)alkyl, (C₂-C₉)heterocycle(C₁-C₆)alkyl, (C₆-C₁₀)aryl(C₁-C₆)alkyl, and (C₁-C₉)heteroaryl(C₁-C₆)alkyl. In addition, the substituent is itself optionally substituted by a further substituent. In one particular embodiment, examples of the further substituent include, but are not limited to, hydrogen, protium, deuterium, tritium, halogen, cyano, nitro, ═S, ═O, N₃, trifluoromethyl, hydroxy, phosphorous-containing group, silicon-containing group, thio, amino, carboxyl, sulfonyl, sulfinyl, (C₁-C₆)acyl, (C₁-C₆)thioacyl, (C₁-C₆)alkyl, (C₂-C₆)alkenyl, (C₂-C₆)alkynyl, (C₃-C₁₀)carbocycle, (C₂-C₉)heterocycle, (C₆-C₁₀)aryl, (C₁-C₉)heteroaryl, trifluoromethyl(C₁-C₆)alkyl, cyano(C₁-C₆)alkyl, halo(C₁-C₆)alkyl, nitro(C₁-C₆)alkyl, hydroxy(C₁-C₆)alkyl, (C₁-C₆)alkoxy, (C₁-C₆)alkylthio, thio(C₁-C₆)alkyl, amino(C₁-C₆)alkyl, (C₁-C₆)alkylamino, ((C₁-C₆)alkyl)₂amino, (C₁-C₆)acyl(C₁-C₆)alkyl, (C₁-C₆)alkylsulfonyl, (C₁-C₆)alkylsulfinyl, hydroxysulfonyl, hydroxysulfinyl, (C₃-C₁₀)carbocycle(C₁-C₆)alkyl, (C₂-C₉)heterocycle(C₁-C₆)alkyl, (C₆-C₁₀)aryl(C₁-C₆)alkyl, and (C₁-C₉)heteroaryl(C₁-C₆)alkyl.

As used herein, the term “formula” may be hereinafter referred to as a “compound(s) of the invention”. Such terms are also defined to include all forms of the compound of formula, including hydrates, solvates, isomers, crystalline and non-crystalline forms, isomorphs, polymorphs, and metabolites thereof. For example, the compounds of formula, or pharmaceutically acceptable salts thereof, may exist in unsolvated and solvated forms. When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

The compounds of “formula” may have asymmetric carbon atoms. The carbon-carbon bonds of the compounds of formula may be depicted herein using a solid line, a solid wedge, or a dotted wedge. The use of a solid line to depict bonds to asymmetric carbon atoms may be meant to indicate that all possible stereoisomers (e.g. specific enantiomers, racemic mixtures, etc.) at that carbon atom are included. The use of either a solid or dotted wedge to depict bonds to asymmetric carbon atoms may be meant to indicate that only the stereoisomer shown is meant to be included. It is possible that compounds of the present application may contain more than one asymmetric carbon atom. In those compounds, the use of a solid line to depict bonds to asymmetric carbon atoms may be meant to indicate that all possible stereoisomers are meant to be included. For example, unless stated otherwise, it may be intended that the compounds of formula can exist as enantiomers and diastereomers or as racemates and mixtures thereof. The use of a solid line to depict bonds to one or more asymmetric carbon atoms in a compound of formula and the use of a solid or dotted wedge to depict bonds to other asymmetric carbon atoms in the same compound may be meant to indicate that a mixture of diastereomers is present.

The compounds of the present application (e.g., the compounds of formula) may exist as clathrates or other complexes. Included within the scope of the invention are complexes such as clathrates, drug-host inclusion complexes wherein, in contrast to the aforementioned solvates, the drug and host may be present in stoichiometric or non-stoichiometric amounts. Also included may be complexes of formula containing two or more organic and/or inorganic components which may be in stoichiometric or non-stoichiometric amounts. The resulting complexes may be ionized, partially ionized, or non-ionized. For a review of such complexes, see J. Pharm. Sci., 64 (8), 1269-1288 by Haleblian (August 1975).

Stereoisomers of formula may include cis and trans isomers, optical isomers such as R and S enantiomers, diastereomers, geometric isomers, rotational isomers, conformational isomers, and tautomers of the compounds of formula, including compounds exhibiting more than one type of isomerism; and mixtures thereof (such as racemates and diastereomeric pairs). Also included may be acid addition or base addition salts wherein the counterion is optically active, for example, D-lactate or L-lysine, or racemic, for example, DL-tartrate or DL-arginine.

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

The compounds of formula may exhibit the phenomena of tautomerism and structural isomerism. For example, the compounds of formula may exist in several tautomeric forms, including the enol and imine forms, and the keto and enamine forms, and geometric isomers and mixtures thereof. All such tautomeric forms may be included within the scope of compounds of formula. Tautomers may exist as mixtures of a tautomeric set in solution. In solid form, usually one tautomer predominates. Even though one tautomer may be described, the present invention includes all tautomers of the compounds of formula.

The present invention also includes isotopically-labeled compounds, which are identical to those recited in formula above, but for the fact that one or more atoms may be replaced by an atom having an atomic mass or mass number different from the atomic mass or mass number usually found in nature. Examples of isotopes that may be incorporated into compounds of formula include isotopes of hydrogen, carbon, nitrogen, oxygen, phosphorus, fluorine and chlorine, such as, but not limited to, ²H, ³H, ¹³C, ¹⁴C, ⁵N, ¹⁸O, ¹⁷O, ³¹P, ³²P, ³⁵S, ¹⁸F, and ³⁶Cl. Certain isotopically-labeled compounds of formula, for example those into which radioactive isotopes such as ³H and ¹⁴C are incorporated, may be useful in drug and/or substrate tissue distribution assays. Tritiated, i.e., ³H, and carbon-14, i.e., ¹⁴C, isotopes may be particularly used for their ease of preparation and detectability. Further, substitution with heavier isotopes such as deuterium, i.e., ²H, may afford certain therapeutic advantages resulting from greater metabolic stability, for example increased in vivo half-life or reduced dosage requirements and, hence, may be used in some circumstances. Isotopically-labeled compounds of formula may generally be prepared by carrying out the procedures disclosed in the Schemes and/or in the Examples below, by substituting an isotopically-labeled reagent for a non-isotopically-labeled reagent.

The compounds of the present application may be used in the form of salts derived from inorganic or organic acids. Depending on the particular compound, a salt of the compound may be advantageous due to one or more of the salt's physical properties, such as enhanced pharmaceutical stability in differing temperatures and humidity, or a desirable solubility in water or oil. In some instances, a salt of a compound also may be used as an aid in the isolation, purification, and/or resolution of the compound.

### Compounds

In one aspect, the present application provides a compound having the structure of formula (I),

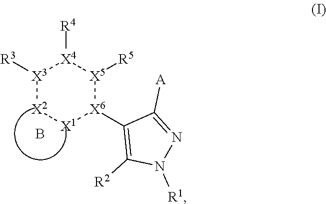

or a pharmaceutically acceptable salt, or prodrug thereof, or a solvate or hydrate of any of the forgoing,
wherein,
each A and B may be independently selected from the group consisting of optionally substituted C₆-C₁₄ aryl, and optionally substituted C₂-C₉ heteroaryl;
each dashed line ( [symbol] ) represents a single or double bond, each X¹, X², X³, X⁴, X⁵ and X⁶ may be independently selected from the group consisting of C, N, and optionally substituted CH;
R¹ may be selected from the group consisting of hydrogen, protium, deuterium, tritium, halogen, cyano, nitro, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁a)carbocycle, optionally substituted (C₂-C₉)heterocycle, optionally substituted (C₆-C₁₀)aryl, and optionally substituted (C₁-C₉)heteroaryl;
R² may be selected from the group consisting of hydrogen, protium, deuterium, tritium, halogen, cyano, nitro, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl,
or R¹ and R² combined with the atoms to which they are attached form an optionally substituted ring;
each R³, R⁴ and R⁵ may be independently absent or may be independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl,
or R³ and R⁴ combined with the atoms to which they are attached may form an optionally substituted ring,
or R³ and R⁵ combined with the atoms to which they are attached may form an optionally substituted ring,
or R⁴ and R⁵ combined with the atoms to which they are attached may form an optionally substituted ring.

In some embodiments, B may be optionally substituted C₂-C₉ heteroaryl.

In some embodiments, B may be selected from the group consisting of optionally substituted pyrazole, optionally substituted imidazole, optionally substituted thiophene, optionally substituted pyrrole, and optionally substituted triazole.

In some embodiments, B may be optionally substituted imidazole.

In some embodiments, B may be substituted with one or more R⁶, each R⁶ may be independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl. For example, B may be substituted with 1, 2, 3, 4, 5, 6, 7, 8, or 9 R⁶. For example, B may be substituted with 1, 2, 3, 4, or 5 R⁶. For example, B may be substituted with 1, 2, or 3 R⁶. For example, B may be substituted with one R⁶. For example, B may be substituted with 2 R⁶. For example, B may be substituted with 3 R⁶.

In some embodiments, each R⁶ may be independently selected from the group consisting of hydrogen, halogen, ═O, optionally substituted (C₁-C₆)alkyl, optionally substituted (C₁-C₆)acyl, and optionally substituted amino.

In some embodiments, each R⁶ may be independently selected from the group consisting of optionally substituted methyl, optionally substituted ethyl and optionally substituted isopropyl.

In some embodiments, B may be optionally substituted imidazole, B may be substituted with one or more R⁶, each R⁶ may be independently selected from the group consisting of optionally substituted methyl, optionally substituted ethyl and optionally substituted isopropyl.

In some embodiments, each R⁶ may be independently substituted with one or more R⁷, each R⁷ may be independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl. For example, R⁶ may be substituted with 1, 2, 3, 4, 5, 6, 7, 8, or 9 R⁷. For example, R⁶ may be substituted with 1, 2, 3, 4, or 5 R⁷. For example, R⁶ may be substituted with 1, 2, or 3 R⁷. For example, R⁶ may be substituted with one R⁷. For example, R⁶ may be substituted with 2 R⁷. For example, R⁶ may be substituted with 3 R⁷.

In some embodiments, each R⁷ may be independently selected from the group consisting of hydrogen, optionally substituted (C₁-C₆)alkyl, optionally substituted (C₁-C₆)acyl, optionally substituted amino, and optionally substituted hydroxy.

In some embodiments, B may be optionally substituted imidazole, B may be substituted with one or more R⁶, each R⁶ may be independently selected from the group consisting of optionally substituted methyl, optionally substituted ethyl and optionally substituted isopropyl, each R⁶ may be independently substituted with one or more R⁷, each R⁷ may be independently selected from the group consisting of hydrogen, optionally substituted (C₁-C₆)alkyl, optionally substituted (C₁-C₆)acyl, optionally substituted amino, and optionally substituted hydroxy.

In some embodiments, each R⁷ may be independently substituted with one or more R⁸, each R⁸ may be independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl. For example, R⁷ may be substituted with 1, 2, 3, 4, 5, 6, 7, 8, or 9 R⁸. For example, R⁷ may be substituted with 1, 2, 3, 4, or 5 R⁸. For example, R⁷ may be substituted with 1, 2, or 3 R⁸. For example, R⁷ may be substituted with one R⁸. For example, R⁷ may be substituted with 2 R⁸. For example, R⁷ may be substituted with 3 R⁸.

In some embodiments, each R⁸ may be independently selected from the group consisting of hydrogen, optionally substituted (C₁-C₆)alkyl, optionally substituted (C₃-C₁₀)carbocycle, and optionally substituted (C₁-C₆)acyl.

In some embodiments, each R⁸ may be independently selected from the group consisting of optionally substituted methyl and optionally substituted cyclopropyl.

In some embodiments, B may be optionally substituted imidazole, B may be substituted with one or more R⁶, each R⁶ may be independently selected from the group consisting of optionally substituted methyl, optionally substituted ethyl and optionally substituted isopropyl, each R⁶ may be independently substituted with one or more R⁷, each R⁷ may be independently selected from the group consisting of hydrogen, optionally substituted (C₁-C₆)alkyl, optionally substituted (C₁-C₆)acyl, optionally substituted (C₁-C₆)thioacyl, optionally substituted amino, and optionally substituted hydroxy, each R⁷ may be independently substituted with one or more R⁸, each R⁸ may be independently selected from the group consisting of optionally substituted methyl and optionally substituted cyclopropyl.

In some embodiments, each R⁸ may be independently substituted with one or more R⁹, each R⁹ may be independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl. For example, R⁸ may be substituted with 1, 2, 3, 4, 5, 6, 7, 8, or 9 R⁹. For example, R⁸ may be substituted with 1, 2, 3, 4, or 5 R⁹. For example, R⁸ may be substituted with 1, 2, or 3 R⁹. For example, R⁸ may be substituted with one R⁹. For example, R⁸ may be substituted with 2 R⁹. For example, R⁸ may be substituted with 3 R⁹.

In some embodiments, each R⁹ may be independently selected from the group consisting of hydrogen, halogen and optionally substituted (C₁-C₆)alkyl.

In some embodiments, B may be optionally substituted imidazole, B may be substituted with one or more R⁶, each R⁶ may be independently selected from the group consisting of optionally substituted methyl, optionally substituted ethyl and optionally substituted isopropyl, each R⁶ may be independently substituted with one or more R⁷, each R⁷ may be independently selected from the group consisting of hydrogen, optionally substituted (C₁-C₆)alkyl, optionally substituted (C₁-C₆)acyl, optionally substituted (C₁-C₆)thioacyl, optionally substituted amino, and optionally substituted hydroxy, each Rt may be independently substituted with one or more R⁸, each R⁸ may be independently selected from the group consisting of optionally substituted methyl and optionally substituted cyclopropyl, each R⁸ may be independently substituted with one or more R⁹, each R⁹ may be independently selected from the group consisting of hydrogen, halogen and optionally substituted (C₁-C₆)alkyl.

In some embodiments, each X¹ and X² may be independently selected from the group consisting of C and N.

In some embodiments, X¹ may be C and X² may be N. In some embodiments, X¹ may be N and X² may be C. In some embodiments, X¹ may be C and X² may be C. In some embodiments, X¹ may be N and X² may be N.

In some embodiments, the compound has the structure selected from the group consisting of

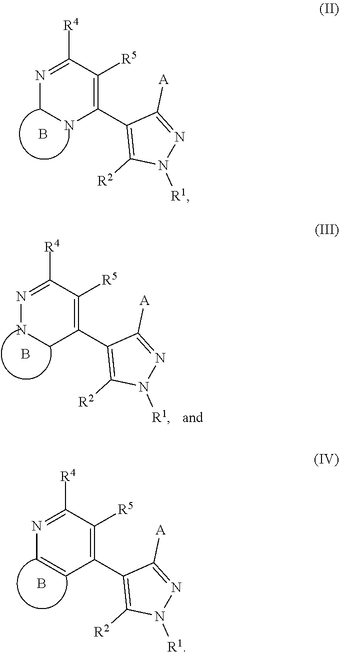

In some embodiments, each R⁴ and R⁵ may be independently selected from the group consisting of hydrogen, protium, deuterium, tritium, halogen, cyano, nitro, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl.

In some embodiments, each R⁴ and R⁵ may be independently selected from the group consisting of hydrogen and halogen.

In some embodiments, R⁴ may be hydrogen and R⁵ may be hydrogen. In some embodiments, R⁴ may be hydrogen and R⁵ may be F or Cl.

In some embodiments, R¹ may be optionally substituted (C₁-C₆)alkyl.

In some embodiments, R¹ may be optionally substituted methyl.

In some embodiments, R² may be hydrogen.

In some embodiments, R¹ and R² combined with the atoms to which they are attached form an optionally substituted ring C, said ring C may be selected from the group consisting of optionally substituted (C₃-C₁₀) carbocycle, optionally substituted (C₂-C₉) heterocycle, optionally substituted (C₆-C₁₀) aryl, and optionally substituted (C₁-C₉) heteroaryl.

In some embodiments, ring C may be optionally substituted (C₂-C₉) heterocycle.

In some embodiments, ring C may be optionally substituted piperazine.

In some embodiments, ring C may be substituted with one or more R¹⁰, each R¹⁰ may be independently absent or may be independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl. For example, ring C may be substituted with 1, 2, 3, 4, 5, 6, 7, 8, or 9 R¹⁰. For example, ring C may be substituted with 1, 2, 3, 4, or 5 R¹⁰. For example, ring C may be substituted with 1, 2, or 3 R¹⁰. For example, ring C may be substituted with one R¹⁰. For example, ring C may be substituted with 2 R¹⁰. For example, ring C may be substituted with 3 R¹⁰.

In some embodiments, each R¹⁰ may be independently selected from the group consisting of optionally substituted (C₁-C₆)acyl and optionally substituted (C₁-C₆)alkyl.

In some embodiments, A may be optionally substituted C₆-C₁₄ aryl.

In some embodiments, A may be optionally substituted phenyl.

In some embodiments, A may be substituted with one or more R¹¹, each R¹¹ may be independently absent or may be independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl. For example, A may be substituted with 1, 2, 3, 4, 5, 6, 7, 8, or 9 R¹¹. For example, A may be substituted with 1, 2, 3, 4, or 5 R¹¹. For example, A may be substituted with 1, 2, or 3 R¹¹. For example, A may be substituted with one R¹¹. For example, A may be substituted with 2 R¹¹. For example, A may be substituted with 3 R¹¹.

In some embodiments, each R¹¹ may independently be halogen. In some embodiments, each R¹¹ may independently be F or Cl.

In some embodiments, each R¹¹ may independently be F.

In some embodiments, A may be optionally substituted phenyl, A may be substituted with one or more R¹¹, each R¹¹ may independently be F.

In some embodiments, the present application provides a compound having the structure of formula (III),

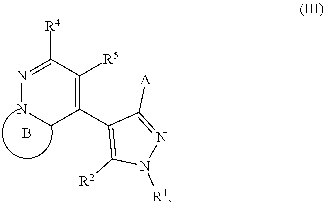

B may be optionally substituted imidazole, B may be substituted with one or more R⁶, each R⁶ may be independently selected from the group consisting of optionally substituted methyl, optionally substituted ethyl and optionally substituted isopropyl, each R⁶ may independently be substituted with one or more R⁷, each R⁷ may be independently selected from the group consisting of hydrogen, optionally substituted (C₁-C₆)alkyl, optionally substituted (C₁-C₆)acyl, optionally substituted amino, and optionally substituted hydroxy, each R⁷ may be independently substituted with one or more R⁸, each R⁸ may be independently selected from the group consisting of optionally substituted methyl and optionally substituted cyclopropyl, each R⁸ may be independently substituted with one or more R⁹, each R⁹ may be independently selected from the group consisting of hydrogen, halogen and optionally substituted (C₁-C₆)alkyl, R⁴ may be hydrogen and R⁵ may be hydrogen, R¹ may be optionally substituted methyl, R² may be hydrogen, A may be optionally substituted phenyl, A may be substituted with one or more R¹¹, R¹¹ may be F.

In some embodiments, the present application provides a compound having the structure of formula (III),

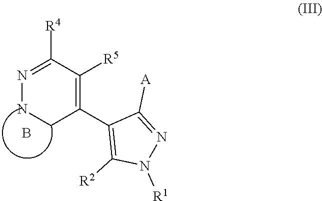

B may be optionally substituted imidazole, B may be substituted with one or more R⁶, each R⁶ may be independently selected from the group consisting of optionally substituted methyl, optionally substituted ethyl and optionally substituted isopropyl, each R⁶ may be independently substituted with one or more R⁷, each R⁷ may be independently selected from the group consisting of optionally substituted amino, and optionally substituted hydroxy, R⁴ may be hydrogen and R⁵ may be hydrogen, R¹ may be optionally substituted methyl, R² may be hydrogen, A may be optionally substituted phenyl, A may be substituted with one or more R¹¹, R¹¹ may be F.

In some cases, the compound may be one of the compounds in Table 1.

TABLE 1

| Com- | | |
|---|---|---|
| pound | Structure | IUPAC Name |
| I-1 | 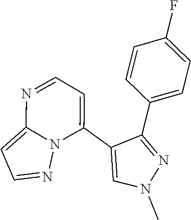 [SMILES: Cn1cc(-c2ccnc3ccnn23)c(-c2ccc(F)cc2)n1] | 7-(3-(4-fluorophenyl)-1-methyl-1H-pyrazol-4-yl)pyrazolo[1,5- a]pyrimidine |
| | I-1 | |
| I-2 | 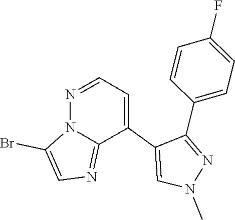 [SMILES: Cn1cc(-c2ccnn3c(Br)cnc23)c(-c2ccc(F)cc2)n1] | 3-bromo-8-(3-(4-fluorophenyl)-1-methyl-1H-pyrazol-4-yl)imi- dazo[1,2-b]pyridazine |
| | I-2 | |
| I-3 | 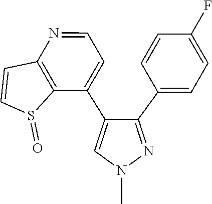 [SMILES: Cn1cc(-c2ccnc3c2S(=O)C=C3)c(-c2ccc(F)cc2)n1] | 7-(3-(4-fluorophenyl)-1-methyl-1H-pyrazol-4-yl)thieno[3,2-b] pyridine 1-oxide |
| | I-3 | |
| I-4 | 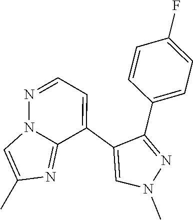 [SMILES: Cc1cn2nccc(-c3cn(C)nc3-c3ccc(F)cc3)c2n1] | 8-(3-(4-fluorophenyl)-1-methyl-1H-pyrazol-4-yl)-2-methyli- midazo[1,2-b]pyridazine |
| | I-4 | |
| I-5 | 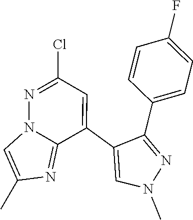 [SMILES: Cc1cn2nc(Cl)cc(-c3cn(C)nc3-c3ccc(F)cc3)c2n1] | 6-chloro-8-(3-(4-fluorophenyl)-1-methyl-1H-pyrazol-4-yl)-2- methylimidazo[1,2-b]pyridazine |
| | I-5 | |
| I-6 | 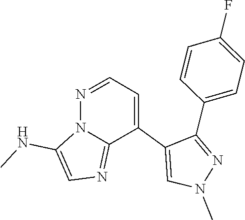 [SMILES: CNc1cnc2c(-c3cn(C)nc3-c3ccc(F)cc3)ccnn12] | 8-(3-(4-fluorophenyl)-1-methyl-1H-pyrazol-4-yl)-N-methyli- midazo[1,2-b]pyridazin-3-amine |
| | I-6 | |
| I-7 | 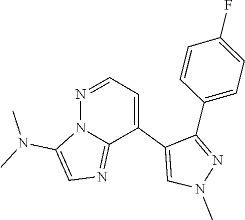 [SMILES: CN(C)c1cnc2c(-c3cn(C)nc3-c3ccc(F)cc3)ccnn12] | 8-(3-(4-fluorophenyl)-1-methyl-1H-pyrazol-4-yl)-N,N-dimeth- ylimidazo[1,2-b]pyridazin-3-amine |
| | I-7 | |
| I-8 | 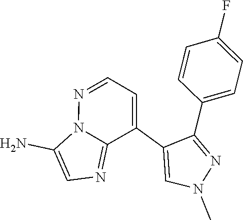 [SMILES: Cn1cc(-c2ccnn3c(N)cnc23)c(-c2ccc(F)cc2)n1] | 8-(3-(4-fluorophenyl)-1-methyl-1H-pyrazol-4-yl)imidazo[1,2- b]pyridazin-3-amine |
| | I-8 | |
| I-9 | 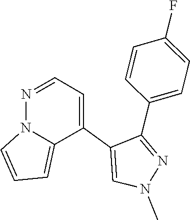 [SMILES: Cn1cc(-c2ccnn3cccc23)c(-c2ccc(F)cc2)n1] | 4-(3-(4-fluorophenyl)-1-methyl-1H-pyrazol-4-yl)pyrrolo[1,2- b]pyridazine |
| | I-9 | |
| I-10 | 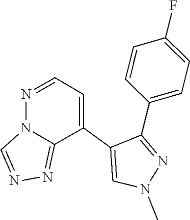 [SMILES: Cn1cc(-c2ccnn3cnnc23)c(-c2ccc(F)cc2)n1] | 8-(3-(4-fluorophenyl)-1-methyl-1H-pyrazol-4-yl)-[1,2,4]triazo- lo[4,3-b]pyridazine |
| | I-10 | |
| I-11 | 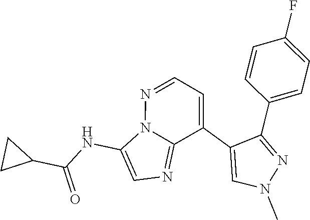 [SMILES: Cn1cc(-c2ccnn3c(NC(=O)C4CC4)cnc23)c(-c2ccc(F)cc2)n1] | N-(8-(3-(4-fluorophenyl)-1-methyl-1H-pyrazol-4-yl)imidazo [1,2-b]pyridazin-3-yl)cyclopropanecarboxamide |
| | I-11 | |
| I-12 | 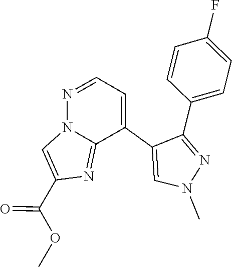 [SMILES: COC(=O)c1cn2nccc(-c3cn(C)nc3-c3ccc(F)cc3)c2n1] | methyl 8-(3-(4-fluorophenyl)-1-methyl-1H-pyrazol-4-yl)imidazo[1,2- b]pyridazine-2-carboxylate |
| | I-12 | |
| I-13 | 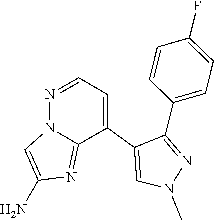 [SMILES: Cn1cc(-c2ccnn3cc(N)nc23)c(-c2ccc(F)cc2)n1] | 8-(3-(4-fluorophenyl)-1-methyl-1H-pyrazol-4-yl)imidazo[1,2- b]pyridazin-2-amine |
| | I-13 | |
| I-14 | 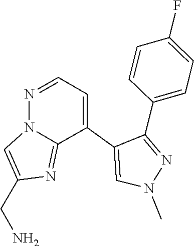 [SMILES: Cn1cc(-c2ccnn3cc(CN)nc23)c(-c2ccc(F)cc2)n1] | (8-(3-(4-fluorophenyl)-1-methyl-1H-pyrazol-4-yl))imidazo[1,2- b]pyridazin-2-yl)methanamine |
| | I-14 | |
| I-15 | 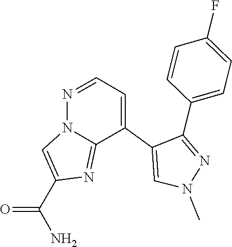 [SMILES: Cn1cc(-c2ccnn3cc(C(N)=O)nc23)c(-c2ccc(F)cc2)n1] | 8-(3-(4-fluorophenyl)-1-methyl-1H-pyrazol-4-yl)imidazo[1,2- b]pyridazine-2-carboxamide |
| | I-15 | |
| I-16 | 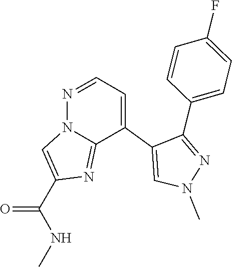 [SMILES: CNC(=O)c1cn2nccc(-c3cn(C)nc3-c3ccc(F)cc3)c2n1] | 8-(3-(4-fluorophenyl)-1-methyl-1H-pyrazol-4-yl)-N-methyli- midazo[1,2-b]pyridazine-2-carboxamide |
| | I-16 | |
| I-17 | 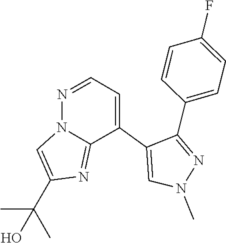 [SMILES: Cn1cc(-c2ccnn3cc(C(C)(C)O)nc23)c(-c2ccc(F)cc2)n1] | 2-(8-(3-(4-fluorophenyl)-1-methyl-1H-pyrazol-4-yl)imidazo[1,2- b]pyridazin-2-yl)propan-2-ol |
| | I-17 | |
| I-18 | 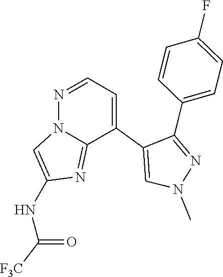 [SMILES: Cn1cc(-c2ccnn3cc(NC(=O)C(F)(F)F)nc23)c(-c2ccc(F)cc2)n1] | 2,2,2-trifluoro-N-(8-(3-(4-fluorophenyl)-1-methyl-1H-pyrazol- 4-yl)imidazo[1,2-b]pyridazin-2-yl)acetamide |
| | I-18 | |
| I-19 | 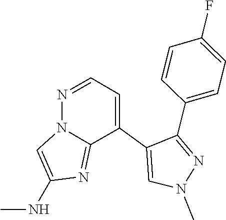 [SMILES: CNc1cn2nccc(-c3cn(C)nc3-c3ccc(F)cc3)c2n1] | 8-(3-(4-fluorophenyl)-1-methyl-1H-pyrazol-4-yl)-N-methyli- midazo[1,2-b]pyridazin-2-amine |
| | I-19 | |
| I-20 | 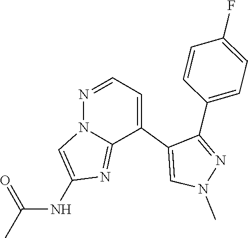 [SMILES: CC(=O)Nc1cn2nccc(-c3cn(C)nc3-c3ccc(F)cc3)c2n1] | N-(8-(3-(4-fluorophenyl)-1-methyl-1H-pyrazol-4-yl)imidazo [1,2-b]pyridazin-2-yl)acetamide |
| | I-20 | |
| I-21 | 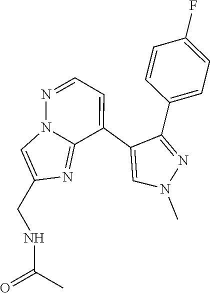 [SMILES: CC(=O)NCc1cn2nccc(-c3cn(C)nc3-c3ccc(F)cc3)c2n1] | N-((8-(3-(4-fluorophenyl)-1-methyl-1H-pyrazol-4-yl)imidazo [1,2-b]pyridazin-2-yl)methyl)acetamide |
| | I-21 | |
| I-22 | 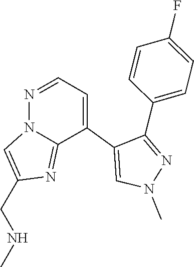 [SMILES: CNCc1cn2nccc(-c3cn(C)nc3-c3ccc(F)cc3)c2n1] | 1-(8-(3-(4-fluorophenyl)-1-methyl-1H-pyrazol-4-yl)imidazo[1,2- b]pyridazin-2-yl)-N-methylmethanamine |
| | I-22 | |
| I-23 | 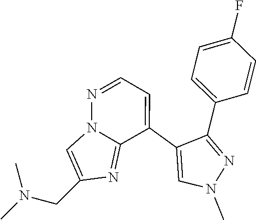 [SMILES: CN(C)Cc1cn2nccc(-c3cn(C)nc3-c3ccc(F)cc3)c2n1] | 1-(8-(3-(4-fluorophenyl)-1-methyl-1H-pyrazol-4-yl)imidazo[1,2- b]pyridazin-2-yl)-N,N-dimethylmethanamine |
| | I-23 | |
| I-24 | 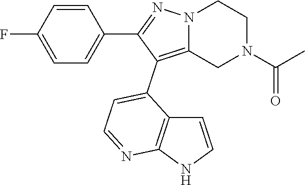 [SMILES: CC(=O)N1CCn2nc(-c3ccc(F)cc3)c(-c3ccnc4[nH]ccc34)c2C1] | 1-(2-(4-fluorophenyl)-3-(1H-pyrrolo[2,3-b]pyridin-4-yl)-6,7- dihydropyrazolo[1,5-a]pyrazin-5(4H)-yl)ethan-1-one |
| | I-24 | |
| I-25 | 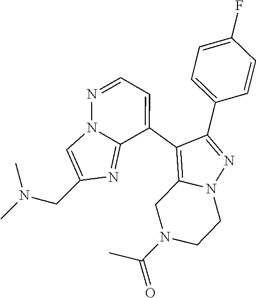 [SMILES: CC(=O)N1CCn2nc(-c3ccc(F)cc3)c(-c3ccnn4cc(CN(C)C)nc34)c2C1] | 1-(3-(2-((dimethylamino)methyl)imidazo[1,2-b]pyridazin-8- yl)-2-(4-fluorophenyl)-6,7-dihydropyrazolo[1,5-a]pyrazin-5(4H)- yl)ethan-1-one |
| | I-25 | |
| I-26 | 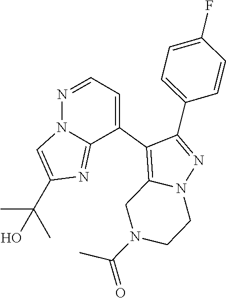 [SMILES: CC(=O)N1CCn2nc(-c3ccc(F)cc3)c(-c3ccnn4cc(C(C)(C)O)nc34)c2C1] | 1-(2-(4-fluorophenyl)-3-(2-(2-hydroxypropan-2-yl)imidazo[1,2- b]pyridazin-8-yl)-6,7-dihydropyrazolo[1,5-a]pyrazin-5(4H)- yl)ethan-1-one |
| | I-26 | |
| I-27 | 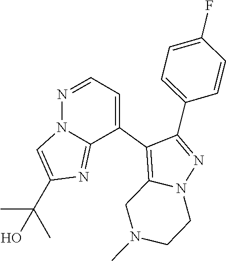 [SMILES: CN1CCn2nc(-c3ccc(F)cc3)c(-c3ccnn4cc(C(C)(C)O)nc34)c2C1] | 2-(8-(2-(4-fluorophenyl)-5-methyl-4,5,6,7-tetrahydropyrazolo [1,5-a]pyrazin-3-yl)imidazo[1,2-b]pyridazin-2-yl)propan-2- ol |
| | I-27 | |
| I-28 | 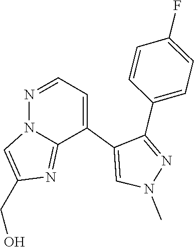 [SMILES: Cn1cc(-c2ccnn3cc(CO)nc23)c(-c2ccc(F)cc2)n1] | (8-(3-(4-fluorophenyl)-1-methyl-1H-pyrazol-4-yl)imidazo[1,2- b]pyridazin-2-yl)methanol |
| | I-28 | |
| I-29 | 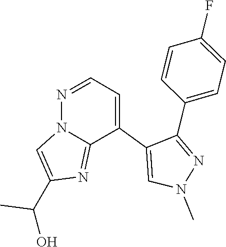 [SMILES: CC(O)c1cn2nccc(-c3cn(C)nc3-c3ccc(F)cc3)c2n1] | 1-(8-(3-(4-fluorophenyl)-1-methyl-1H-pyrazol-4-yl)imidazo[1,2- b]pyridazin-2-yl)ethan-1-ol |
| | I-29 | |
| I-30 | 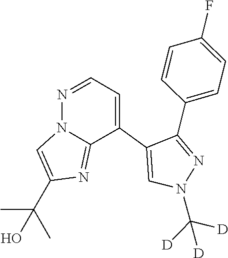 [SMILES: [2H]C([2H])([2H])n1cc(-c2ccnn3cc(C(C)(C)O)nc23)c(-c2ccc(F)cc2)n1] | 2-(8-(3-(4-fluorophenyl)-1-(methyl-d3)-1H-pyrazol-4-yl)imi- dazo[1,2-b]pyridazin-2-yl)propan-2-ol |
| | I-30 | |
| I-31 | 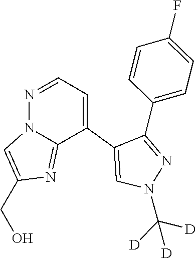 [SMILES: [2H]C([2H])([2H])n1cc(-c2ccnn3cc(CO)nc23)c(-c2ccc(F)cc2)n1] | (8-(3-(4-fluorophenyl)-1-(methyl-d3)-1H-pyrazol-4-yl)imida- zo[1,2-b]pyridazin-2-yl)methanol |
| | I-31 | |
| I-32 | 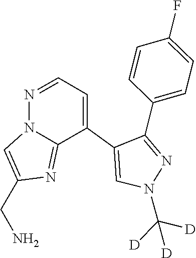 [SMILES: [2H]C([2H])([2H])n1cc(-c2ccnn3cc(CN)nc23)c(-c2ccc(F)cc2)n1] | (8-(3-(4-fluorophenyl)-1-(methyl-d3)-1H-pyrazol-4-yl)imida- zo[1,2-b]pyridazin-2-yl)methanamine |
| | I-32 | |
| I-33 | 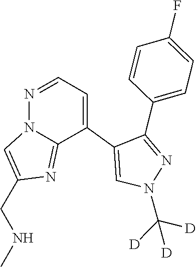 [SMILES: [2H]C([2H])([2H])n1cc(-c2ccnn3cc(CNC)nc23)c(-c2ccc(F)cc2)n1] | 1-(8-(3-(4-fluorophenyl)-1-(methyl-d3)-1H-pyrazol-4-yl)imi- dazo[1,2-b]pyridazin-2-yl)-N-methylmethanamine |
| | I-33 | |
| I-34 | 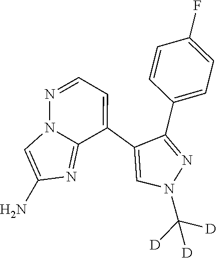 [SMILES: [2H]C([2H])([2H])n1cc(-c2ccnn3cc(N)nc23)c(-c2ccc(F)cc2)n1] | 8-(3-(4-fluorophenyl)-1-(methyl-d3)-1H-pyrazol-4-yl)imidazo [1,2-b]pyridazin-2-amine |
| | I-34 | |

### Medical Use

In one aspect, the present application provides a method for inhibiting casein kinase (CK) activity, said method comprising administering to a subject in need thereof an effective amount of the compound of the present application, or a pharmaceutically acceptable salt, prodrug, or metabolite thereof, or a solvate or hydrate of any of the foregoing. For example, the casein kinase (CK) may be selected from the group consisting of casein kinase I alpha (CK1α), casein kinase I delta (CK1δ) and casein kinase I epsilon (CK1ε). For example, the method may be selected from the group consisting of an in vitro method, an ex vivo method, and an in vivo method.

In another embodiment, the present application provides use the compound of the present application, or a pharmaceutically acceptable salt, prodrug, or metabolite thereof, or a solvate or hydrate of any of the foregoing of the present application in the preparation of a drug and/or a kit for use in inhibiting casein kinase (CK) activity. For example, the casein kinase (CK) may be selected from the group consisting of casein kinase I alpha (CK1α), casein kinase I delta (CK1δ) and casein kinase I epsilon (CK1ε). For example, the method may be selected from the group consisting of an in vitro method, an ex vivo method, and an in vivo method.

In another embodiment, the present application provides the compound of the present application, or a pharmaceutically acceptable salt, prodrug, or metabolite thereof, or a solvate or hydrate of any of the foregoing of the present application for use in inhibiting casein kinase (CK) activity. For example, the casein kinase (CK) may be selected from the group consisting of casein kinase I alpha (CK1α), casein kinase I delta (CK1δ) and casein kinase I epsilon (CK1ε). For example, the method may be selected from the group consisting of an in vitro method, an ex vivo method, and an in vivo method.

In another aspect, the present application provides a method for preventing and/or treating a disease or disorder, said method comprising administering to a subject in need thereof an effective amount of the compound of the present application, or a pharmaceutically acceptable salt, prodrug, or metabolite thereof, or a solvate or hydrate of any of the foregoing. For example, the disease or disorder may be selected from the group consisting of neurological disease and psychiatric disease. For example, the disease or disorder may be selected from the group consisting of mood disorder, sleep disorder, and circadian disorder. For example, the disease or disorder may be selected from the group consisting of depressive disorder and bipolar disorder.

In another embodiment, the present application provides use the compound of the present application, or a pharmaceutically acceptable salt, prodrug, or metabolite thereof, or a solvate or hydrate of any of the foregoing of the present application in the preparation of a drug and/or a kit for use in preventing and/or treating a disease or disorder. For example, the disease or disorder may be selected from the group consisting of neurological disease and psychiatric disease. For example, the disease or disorder may be selected from the group consisting of mood disorder, sleep disorder, and circadian disorder. For example, the disease or disorder may be selected from the group consisting of depressive disorder and bipolar disorder.

In another embodiment, the present application provides the compound of the present application, or a pharmaceutically acceptable salt, prodrug, or metabolite thereof, or a solvate or hydrate of any of the foregoing of the present application for use in preventing and/or treating a disease or disorder. For example, the disease or disorder may be selected from the group consisting of neurological disease and psychiatric disease. For example, the disease or disorder may be selected from the group consisting of mood disorder, sleep disorder, and circadian disorder. For example, the disease or disorder may be selected from the group consisting of depressive disorder and bipolar disorder.

In another embodiment, the present application provides compositions comprising a compound of the present application or a pharmaceutically acceptable salt, prodrug, or metabolite thereof, or a solvate or hydrate of any of the foregoing, and optionally a pharmaceutically acceptable carrier.

The compounds of the application may be administered orally. Oral administration may involve swallowing, so that the compound enters the gastrointestinal tract, or buccal or sublingual administration may be employed by which the compound enters the blood stream directly from the mouth.

In some cases, the compounds of the present application may also be administered directly into the blood stream, into muscle, or into an internal organ. Suitable means for parenteral administration may include intravenous, intraarterial, intraperitoneal, intrathecal, intraventricular, intraurethral, intrasternal, intracranial, intramuscular and subcutaneous. Suitable devices for parenteral administration may include needle (including microneedle) injectors, needle-free injectors and infusion techniques.

The compounds of the present application may also be administered topically to the skin or mucosa, that is, dermally or transdermally. In some cases, the compounds of the present application may also be administered intranasally or by inhalation. In some cases, the compounds of the present application may be administered rectally or vaginally. In another embodiment, the compounds of the present application may also be administered directly to the eye or ear.

The dosage regimen for the compounds and/or compositions containing the compounds is based on a variety of factors, including the type, age, weight, sex and medical condition of the patient; the severity of the condition; the route of administration; and the activity of the particular compound employed. Thus, the dosage regimen may vary widely. Dosage levels of the order from about 0.01 mg to about 100 mg per kilogram of body weight per day may be useful in the treatment of the above-indicated conditions.

Suitable subjects according to the present invention include mammalian subjects. Mammals according to the present invention may include, but are not limited to, canine, feline, bovine, caprine, equine, ovine, porcine, rodents, lagomorphs, primates, and the like, and encompass mammals in utero. In one embodiment, humans are suitable subjects. Human subjects may be of either gender and at any stage of development.

In another embodiment, the present application provides use of one or more compounds of the present application for the preparation of a medicament for the treatment of the conditions recited herein.

For the treatment of the conditions referred to above, the compounds of the present application may be administered as compound per se. Alternatively, pharmaceutically acceptable salts may be suitable for medical applications because of their greater aqueous solubility relative to the parent compound.

In another embodiment, the present application provides compositions. Such compositions may comprise a compound of the present application presented with a pharmaceutically acceptable carrier. The carrier may be a solid product, a liquid, or both, and may be formulated with the compound as a unit-dose composition, for example, a tablet, which can contain from 0.05% to 95% by weight of the active compounds. A compound of the present application may be coupled with suitable polymers as targetable drug carriers. Other pharmacologically active substances may also be present.

The compounds of the present invention may be administered by any suitable route, maybe in the form of a pharmaceutical composition adapted to such a route, and in a dose effective for the treatment intended. The active compounds and compositions, for example, may be administered orally, rectally, parenterally, or topically.

The compounds of the present application may be used, alone or in combination with other therapeutic agents, in the treatment of various conditions or disease states. The compound(s) of the present application and other therapeutic agent(s) may be administered simultaneously (either in the same dosage form or in separate dosage forms) or sequentially.

The administration of two or more compounds “in combination” may mean that the two compounds are administered closely enough in time that the presence of one alters the biological effects of the other. The two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” may mean that the compounds are administered in combination.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Example 1 Preparation of Compounds

### Example 1-1 Preparation of Compound I-1

FIG. 1 illustrates the synthetic scheme of compound I-1. As shown in FIG. 1, the specific synthesis step is as follows:

### Step 1

To a stirred solution of 3-(4-fluorophenyl)-1-methyl-4-(4,4,5,5-tetramethyl-1,3,2-dioxaborolan-2-yl)pyrazole(236.11 mg, 1.20 equiv) and 7-chloropyrazolo[1,5-a]pyrimidine(100.00 mg, 0.651 mmol, 1.00 equiv) in DMF (2.00 mL) were added Pd(dppf)Cl₂CH₂Cl₂ (53.05 mg, 0.065 mmol, 0.10 equiv) and Cs₂CO₃ (636.49 mg, 1.953 mmol, 3.00 equiv) in portions at room temperature under nitrogen atmosphere. The resulting mixture was stirred for 2 h at 80° C. under nitrogen atmosphere. The reaction was monitored by LCMS. The resulting mixture was concentrated under reduced pressure. The residue was purified by silica gel column chromatography, eluted with EA to afford 3-(4-fluorophenyl)-1-methyl-4-[pyrazolo[1,5-a]pyrimidin-7-yl]pyrazole(68 mg, 35.60%) as a white solid. LC/MS (ESI, m/z): [(M+1)]+=294.1. ¹H NMR (300 MHz, Chloroform-d) δ 8.46 (dd, J=7.3, 0.9 Hz, 1H), 8.10 (d, J=2.3 Hz, 1H), 8.05 (s, 1H), 7.60-7.50 (m, 2H), 7.19-7.08 (m, 2H), 6.65-6.57 (m, 2H), 4.02 (s, 3H).

### Example 1-2 Preparation of Compound I-2

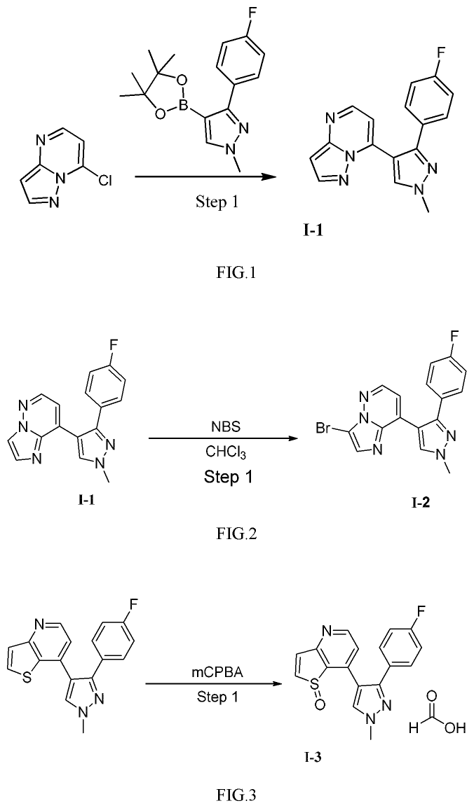

FIG. 2 illustrates the synthetic scheme of compound 1-2. As shown in FIG. 2, the specific synthesis step is as follows:

### Step 1

A solution of 3-(4-fluorophenyl)-4-[imidazo[1,2-b]pyridazin-8-yl]-1-methylpyrazole (50 mg, 0.17 mmol, 1.0 equiv) and NBS (32 mg, 0.18 mmol, 1.1 equiv) in CHCl₃ (1.00 mL) was stirred for 30 min at 60° C. under nitrogen atmosphere. The resulting mixture was diluted with sat.Na₂CO₃ aq. (10 mL). The resulting mixture was extracted with CHCl₃ (3×10 mL). The combined organic layers were washed with brine (2×25 mL), dried over anhydrous Na₂SO₄. After filtration, the filtrate was concentrated under reduced pressure. The residue was purified by reverse flash chromatography with the following conditions: column, C18 silica gel; mobile phase, ACN in water, 5% to 95% gradient in 20 min; detector, UV 254 nm. The fractions containing desired product were concentrated to afford 4-[3-bromoimidazo[1,2-b]pyridazin-8-yl]-3-(4-fluorophenyl)-1-methylpyrazole (43.8 mg, 69%) as a white solid. ¹H NMR (300 MHz, CDCl₃) δ 8.70 (s, 1H), 8.22 (d, J=5.0 Hz, 1H), 7.78 (s, 1H), 7.53-7.46 (m, 2H), 7.15-7.07 (m, 2H), 6.78 (d, J=5.0 Hz, 1H), 4.03 (s, 3H). LC/MS (ESI, m/z): [(M+1)]+=372, 374.

### Example 1-3 Preparation of Compound I-3

FIG. 3 illustrates the synthetic scheme of compound I-3. As shown in FIG. 3, the specific synthesis step is as follows:

### Step 1

A solution of 3-(4-fluorophenyl)-1-methyl-4-[thieno[3,2-b]pyridin-7-yl]pyrazole (20 mg, 0.07 mmol, 1.0 equiv) and m-CPBA (17 mg, 0.1 mmol, 1.5 equiv) in DCM (1 mL) was stirred for 16 h at room temperature under nitrogen atmosphere. The resulting mixture was concentrated under reduced pressure. The residue was purified by reverse flash chromatography with the following conditions: column, C18 silica gel; mobile phase, ACN in water, 5% to 40% gradient in 20 min; detector, UV 254 nm. The fractions containing desired product were concentrated to afford 7-[3-(4-fluorophenyl)-1-methylpyrazol-4-yl]-1lambda4-thieno[3,2-b]pyridin-1-one; formic acid (10.7 mg, 45%) as a white solid. ¹H NMR (300 MHz, CDCl₃) δ 8.22 (d, J=6.5 Hz, 1H), 7.91 (d, J=5.7 Hz, 1H), 7.78 (s, 1H), 7.68 (d, J=5.6 Hz, 1H), 7.42-7.34 (m, 2H), 7.03-6.95 (m, 3H), 4.05 (s, 3H). LC/MS (ESI, m/z): [(M+1−FA)]⁺=326.

### Example 1-4 Preparation of Compound I-4

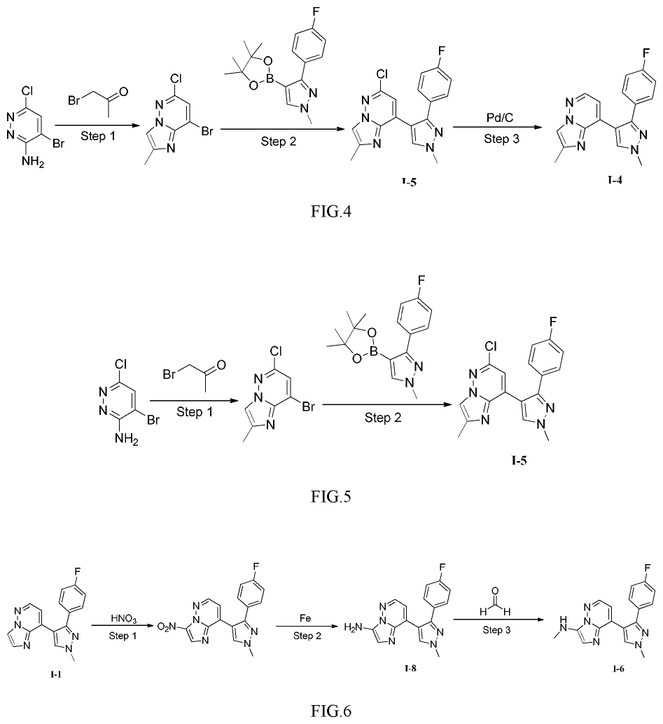

FIG. 4 illustrates the synthetic scheme of compound I-4. As shown in FIG. 4, the specific synthesis steps are as follows:

### Step 1: 8-bromo-6-chloro-2-methylimidazo[1,2-b]pyridazine

To a stirred mixture of 4-bromo-6-chloropyridazin-3-amine (2 g, 9.6 mmol, 1.0 equiv) and bromoacetone (3.9 g, 28 mmol, 3.0 equiv) in IPA (50 mL) was added Na₂CO₃ (3.1 g, 28 mmol, 3.0 equiv) at room temperature under nitrogen atmosphere. The resulting mixture was stirred for 2 h at 90° C. under nitrogen atmosphere. The resulting mixture was concentrated under reduced pressure. The residue was purified by silica gel column chromatography, eluted with PE/EA (3:1) to afford 8-bromo-6-chloro-2-methylimidazo[1,2-b]pyridazine (1.6 g, 68%) as a light yellow solid. ¹HNMR (300 MHz, CDCl₃) δ 7.77 (s, 1H), 7.32 (s, 1H), 2.53 (s, 3H). LC/MS (ESI, m/z): [(M+1)]⁺=246, 248.

### Step 2: 4-[6-chloro-2-methylimidazo[1,2-b]pyridazin-8-yl]-3-(4-fluorophenyl)-1-methylpyrazole

To a stirred mixture of 8-bromo-6-chloro-2-methylimidazo[1,2-b]pyridazine (500 mg, 2 mmol, 1.0 equiv) and 3-(4-fluorophenyl)-1-methyl-4-(4,4,5,5-tetramethyl-1,3,2-dioxaborolan-2-yl)pyrazole (613 mg, 2 mmol, 1.0 equiv) in DMF (8 mL) were added Cs₂CO₃ (1.3 g, 4.1 mmol, 2.0 equiv) and Pd(dppf)Cl₂CH₂Cl₂ (165 mg, 0.2 mmol, 0.1 equiv) at room temperature under nitrogen atmosphere. The resulting mixture was stirred for 2 h at 80° C. under nitrogen atmosphere. The resulting mixture was concentrated under reduced pressure. The residue was purified by silica gel column chromatography, eluted with PE/EA (1:1) to afford 4-[6-chloro-2-methylimidazo[1,2-b]pyridazin-8-yl]-3-(4-fluorophenyl)-1-methylpyrazole (400 mg, 58%) as a light yellow solid. ¹HNMR (400 MHz, CDCl₃) δ 8.77 (s, 1H), 7.71 (s, 1H), 7.53-7.48 (m, 2H), 7.16-7.11 (m, 2H), 6.69 (s, 1H), 4.04 (s, 3H), 2.55 (s, 3H). LC/MS (ESI, m/z): [(M+1)]⁺=342.

### Step 3

To a stirred solution of 4-[6-chloro-2-methylimidazo[1,2-b]pyridazin-8-yl]-3-(4-fluorophenyl)-1-methylpyrazole (100 mg, 0.29 mmol, 1.0 equiv) and TEA (30 mg, 0.29 mmol, 1.0 equiv) in EA (5 mL) was added 10% Pd/C (25 mg) at room temperature under nitrogen atmosphere. The resulting mixture was stirred for 16 h at 50 degrees C. under nitrogen atmosphere. The resulting mixture was filtered and the filter cake was washed with EA (3×5 mL). The filtrate was concentrated under reduced pressure. The residue was purified by reverse flash chromatography with the following conditions: column, C18 silica gel; mobile phase, ACN in 0.1% FA aq., 5% to 40% gradient in 20 min; detector, UV 254 nm. The fractions containing desired product were concentrated to afford 3-(4-fluorophenyl)-1-methyl-4-[2-methylimidazo[1,2-b]pyridazin-8-yl]pyrazole (17.6 mg, 20%) as a white solid. ¹H NMR (300 MHz, CDCl₃) δ 8.70 (s, 1H), 7.99 (d, J=4.9 Hz, 1H), 7.76 (d, J=0.9 Hz, 1H), 7.54-7.47 (m, 2H), 7.14-7.05 (m, 2H), 6.64 (d, J=5.0 Hz, 1H), 4.03 (s, 3H), 2.55 (d, J=0.9 Hz, 3H). LC/MS (ESI, m/z): [(M+1)]⁺=308.

### Example 1-5 Preparation of Compound I-5

FIG. 5 illustrates the synthetic scheme of compound 1-5. As shown in FIG. 5, the specific synthesis steps are as follows:

### Step 1: 8-bromo-6-chloro-2-methylimidazo[1,2-b]pyridazine

To a stirred mixture of 4-bromo-6-chloropyridazin-3-amine (2 g, 9.6 mmol, 1.0 equiv) and bromoacetone (3.9 g, 28 mmol, 3.0 equiv) in IPA (50 mL) was added Na₂CO₃ (3.1 g, 28 mmol, 3.0 equiv) at room temperature under nitrogen atmosphere. The resulting mixture was stirred for 2 h at 90° C. under nitrogen atmosphere. The resulting mixture was concentrated under reduced pressure. The residue was purified by silica gel column chromatography, eluted with PE/EA (3:1) to afford 8-bromo-6-chloro-2-methylimidazo[1,2-b]pyridazine (1.6 g, 68%) as a light yellow solid. ¹HNMR (300 MHz, CDCl₃) δ 7.77 (s, 1H), 7.32 (s, 1H), 2.53 (s, 3H). LC/MS (ESI, m/z): [(M+1)]⁺=246, 248.

### Step 2

To a stirred mixture of 8-bromo-6-chloro-2-methylimidazo[1,2-b]pyridazine (500 mg, 2 mmol, 1.0 equiv) and 3-(4-fluorophenyl)-1-methyl-4-(4,4,5,5-tetramethyl-1,3,2-dioxaborolan-2-yl)pyrazole (613 mg, 2 mmol, 1.0 equiv) in DMF (8 mL) were added Cs₂CO₃ (1.3 g, 4.1 mmol, 2.0 equiv) and Pd(dppf)Cl₂CH₂Cl₂ (165 mg, 0.2 mmol, 0.1 equiv) at room temperature under nitrogen atmosphere. The resulting mixture was stirred for 2 h at 80° C. under nitrogen atmosphere. The resulting mixture was concentrated under reduced pressure. The residue was purified by silica gel column chromatography, eluted with PE/EA (1:1) to afford 4-[6-chloro-2-methylimidazo[1,2-b]pyridazin-8-yl]-3-(4-fluorophenyl)-1-methylpyrazole (400 mg, 58%) as a light yellow solid. ¹HNMR (400 MHz, CDCl₃) δ 8.77 (s, 1H), 7.71 (s, 1H), 7.53-7.48 (m, 2H), 7.16-7.11 (m, 2H), 6.69 (s, 1H), 4.04 (s, 3H), 2.55 (s, 3H). LC/MS (ESI, m/z): [(M+1)]⁺=342.

### Example 1-6 Preparation of Compound I-6

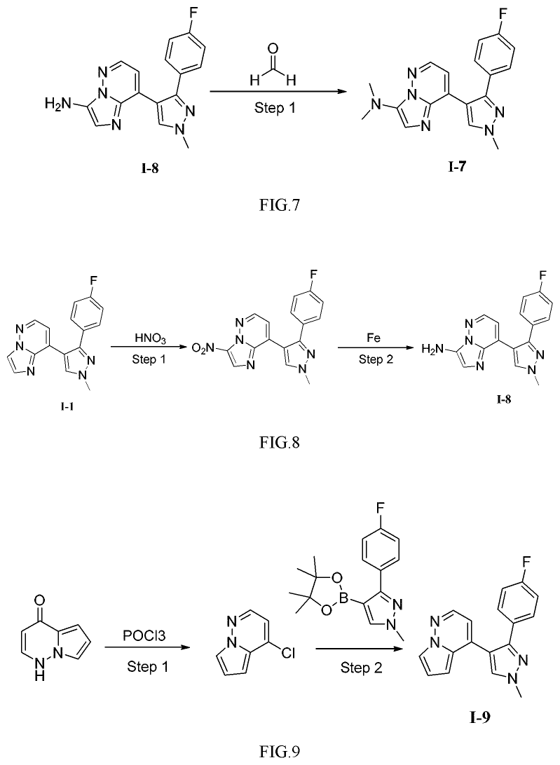

FIG. 6 illustrates the synthetic scheme of compound 1-6. As shown in FIG. 6, the specific synthesis steps are as follows:

### Step 1: 3-(4-fluorophenyl)-1-methyl-4-[3-nitroimidazo[1,2-b]pyridazin-8-yl]pyrazole

To a stirred solution of 3-(4-fluorophenyl)-4-[imidazo[1,2-b]pyridazin-8-yl]-1-methylpyrazole (200 mg, 0.68 mmol, 1.0 equiv) in H₂SO₄ (2 mL) was added HNO₃ (311 mg, 3.4 mmol, 5.0 equiv) dropwise at 0° C. under nitrogen atmosphere. The resulting mixture was stirred for 20 min at room temperature under nitrogen atmosphere. The reaction was quenched by the addition of 8 M NaOH aq (7 mL) at 0° C. The resulting mixture was extracted with EA (3×30 mL). The combined organic layers were washed with brine (2×80 mL), dried over anhydrous Na₂SO₄. After filtration, the filtrate was concentrated under reduced pressure. The residue was purified by silica gel column chromatography, eluted with PE/EA (1:1) to afford 3-(4-fluorophenyl)-1-methyl-4-[3-nitroimidazo[1,2-b]pyridazin-8-yl]pyrazole (200 mg, 87%) as a light yellow solid. ¹H NMR (300 MHz, CDCl₃) δ 8.69-8.65 (m, 1H), 8.30 (dd, J=7.2, 2.2 Hz, 1H), 8.15 (d, J=4.8 Hz, 1H), 8.04 (d, J=1.3 Hz, 1H), 7.83-7.77 (m, 2H), 7.32 (dd, J=10.5, 8.6 Hz, 1H), 6.73 (d, J=4.9 Hz, 1H), 4.06 (s, 3H). LC/MS (ESI, m/z): [(M+1)]⁺=339.

### Step 2: 8-[3-(4-fluorophenyl)-1-methylpyrazol-4-yl]imidazo[1,2-b]pyridazin-3-amine

To a stirred mixture of 3-(4-fluorophenyl)-1-methyl-4-[3-nitroimidazo[1,2-b]pyridazin-8-yl]pyrazole (200 mg, 0.59 mmol, 1.0 equiv) and NH₄Cl (95 mg, 1.8 mmol, 3.0 equiv) in EtOH (5 mL)/H₂O (1 mL) was added Fe (165 mg, 3.0 mmol, 5.0 equiv) at room temperature under nitrogen atmosphere. The resulting mixture was stirred for 1 h at 80° C. under nitrogen atmosphere. The resulting mixture was filtered and the filter cake was washed with EtOH (3×5 mL). The filtrate was concentrated under reduced pressure. The residue was purified by silica gel column chromatography, eluted with DCM/MeOH (10:1) to afford 8-[3-(4-fluorophenyl)-1-methylpyrazol-4-yl]imidazo[1,2-b]pyridazin-3-amine (160 mg, 88%) as a light yellow solid. ¹H NMR (400 MHz, CDCl₃) δ 8.76 (s, 1H), 8.09 (d, J=5.0 Hz, 1H), 7.99 (d, J=1.3 Hz, 1H), 7.78 (d, J=1.3 Hz, 1H), 7.04-6.95 (m, 2H), 6.84-6.80 (m, 2H), 4.02 (s, 3H), 3.81 (br, 2H). LC/MS (ESI, m/z): [(M+1)]⁺=309.

### Step 3: 8-[3-(4-fluorophenyl)-1-methylpyrazol-4-yl]-N-methylimidazo[1,2-b]pyridazin-3-amine

To a stirred solution of 8-[3-(4-fluorophenyl)-1-methylpyrazol-4-yl]imidazo[1,2-b]pyridazin-3-amine (140 mg, 0.45 mmol, 1.0 equiv) and formaldehyde (14 mg, 0.45 mmol, 1.0 equiv) in MeOH (7 mL) was added AcOH (41 mg, 0.68 mmol, 1.5 equiv) at room temperature under nitrogen atmosphere. The resulting mixture was stirred for 30 min at room temperature under nitrogen atmosphere. To the above mixture was added NaBH₃CN (57 mg, 0.91 mmol, 2.0 equiv) at room temperature. The resulting mixture was stirred for additional 1 h at room temperature. The resulting mixture was concentrated under reduced pressure. The residue was purified by Prep-HPLC with the following conditions (Column: X Bridge Prep C18 OBD Column, 19×150 mm 5 um; Mobile Phase A: water (10 mmol/L NH₄HCO₃), Mobile Phase B:ACN; Flow rate: 25 mL/min; Gradient: 24% B to 51% B in 8 min; 220 nm; RT1:7.12 min, Injection Volume: 0.4 ml; Number Of Runs: 8) to afford 8-[3-(4-fluorophenyl)-1-methylpyrazol-4-yl]-N-methylimidazo[1,2-b]pyridazin-3-amine (58 mg, 40%) as an off-white solid. ¹H NMR (400 MHz, CDCl₃) δ 8.79 (s, 1H), 8.07 (d, J=4.9 Hz, 1H), 7.99 (d, J=1.2 Hz, 1H), 7.77 (d, J=1.2 Hz, 1H), 6.99 (dd, J=11.5, 8.2 Hz, 11H), 6.86-6.81 (m, 2H), 6.75 (ddd, J=8.2, 4.6, 2.1 Hz, 1H), 4.03 (s, 3H), 2.83 (s, 3H). LC/MS (ESI, m/z): [(M+1)]⁺=323.

### Example 1-7 Preparation of Compound I-7

FIG. 7 illustrates the synthetic scheme of compound I-7. As shown in FIG. 7, the specific synthesis steps are as follows:

### Step 1

To a stirred solution of 8-[3-(4-fluorophenyl)-1-methylpyrazol-4-yl]imidazo[1,2-b]pyridazin-3-amine (140 mg, 0.45 mmol, 1.0 equiv) and formaldehyde (14 mg, 0.45 mmol, 1.0 equiv) in MeOH (7 mL) was added AcOH (41 mg, 0.68 mmol, 1.5 equiv) at room temperature under nitrogen atmosphere. The resulting mixture was stirred for 30 min at room temperature under nitrogen atmosphere. To the above mixture was added NaBH₃CN (57 mg, 0.91 mmol, 2.0 equiv) at room temperature. The resulting mixture was stirred for additional 1 h at room temperature. The resulting mixture was concentrated under reduced pressure. The residue was purified by Prep-HPLC with the following conditions (Column: X Bridge Prep C18 OBD Column, 19×150 mm 5 um; Mobile Phase A: water (10 mmol/L NH₄HCO₃), Mobile Phase B: ACN; Flow rate: 25 mL/min; Gradient: 24 B to 51 B in 8 min; 220 nm; RT₁:7.68 min; Injection Volume: 0.4 ml; Number Of Runs: 8) to afford 8-[3-(4-fluorophenyl)-1-methylpyrazol-4-yl]-N,N-dimethylimidazo[1,2-b]pyridazin-3-amine (28 mg, 19%) as an off-white solid. ¹H NMR (400 MHz, CDCl₃) δ 8.78 (s, 1H), 8.07 (d, J=4.9 Hz, 1H), 7.99 (d, J=1.2 Hz, 1H), 7.78 (d, J=1.3 Hz, 1H), 7.10-7.04 (m, 2H), 7.03-6.98 (m, 1H), 6.79 (d, J=5.0 Hz, 1H), 4.03 (s, 3H), 2.83 (s, 6H). LC/MS (ESI, m/z): [(M+1)]⁺=337.

### Example 1-8 Preparation of Compound I-8

FIG. 8 illustrates the synthetic scheme of compound I-8. As shown in FIG. 8, the specific synthesis steps are as follows:

### Step 1: 3-(4-fluorophenyl)-1-methyl-4-[3-nitroimidazo[1,2-b]pyridazin-8-yl]pyrazole

To a stirred solution of 3-(4-fluorophenyl)-4-[imidazo[1,2-b]pyridazin-8-yl]-1-methylpyrazole (200 mg, 0.68 mmol, 1.0 equiv) in H₂SO₄ (2 mL) was added HNO₃ (311 mg, 3.4 mmol, 5.0 equiv) dropwise at 0° C. under nitrogen atmosphere. The resulting mixture was stirred for 20 min at room temperature under nitrogen atmosphere. The reaction was quenched by the addition of 8 M NaOH aq (7 mL) at 0° C. The resulting mixture was extracted with EA (3×30 mL). The combined organic layers were washed with brine (2×80 mL), dried over anhydrous Na₂SO₄. After filtration, the filtrate was concentrated under reduced pressure. The residue was purified by silica gel column chromatography, eluted with PE/EA (1:1) to afford 3-(4-fluorophenyl)-1-methyl-4-[3-nitroimidazo[1,2-b]pyridazin-8-yl]pyrazole (200 mg, 87%) as a light yellow solid. ¹H NMR (300 MHz, CDCl₃) δ 8.69-8.65 (m, 1H), 8.30 (dd, J=7.2, 2.2 Hz, 1H), 8.15 (d, J=4.8 Hz, 1H), 8.04 (d, J=1.3 Hz, 1H), 7.83-7.77 (m, 2H), 7.32 (dd, J=10.5, 8.6 Hz, 1H), 6.73 (d, J=4.9 Hz, 1H), 4.06 (s, 3H). LC/MS (ESI, m/z): [(M+1)]⁺=339.

### Step 2

To a stirred mixture of 3-(4-fluorophenyl)-1-methyl-4-[3-nitroimidazo[1,2-b]pyridazin-8-yl]pyrazole (200 mg, 0.59 mmol, 1.0 equiv) and NH₄Cl (95 mg, 1.8 mmol, 3.0 equiv) in EtOH (5 mL)/H₂O (1 mL) was added Fe (165 mg, 3.0 mmol, 5.0 equiv) at room temperature under nitrogen atmosphere. The resulting mixture was stirred for 1 h at 80° C. under nitrogen atmosphere. The resulting mixture was filtered and the filter cake was washed with EtOH (3×5 mL). The filtrate was concentrated under reduced pressure. The residue was purified by silica gel column chromatography, eluted with DCM/MeOH (10:1) to afford 8-[3-(4-fluorophenyl)-1-methylpyrazol-4-yl]imidazo[1,2-b]pyridazin-3-amine (160 mg, 88%) as a light yellow solid. ¹H NMR (400 MHz, CDCl₃) δ 8.76 (s, 1H), 8.09 (d, J=5.0 Hz, 1H), 7.99 (d, J=1.3 Hz, 1H), 7.78 (d, J=1.3 Hz, 1H), 7.04-6.95 (m, 2H), 6.84-6.80 (m, 2H), 4.02 (s, 3H), 3.81 (br, 2H). LC/MS (ESI, m/z): [(M+1)]⁺=309.

### Example 1-9 Preparation of Compound I-9

FIG. 9 illustrates the synthetic scheme of compound I-9. As shown in FIG. 9, the specific synthesis steps are as follows:

### Step 1: 4-chloropyrrolo[1,2-b]pyridazine

Into a 40 mL vial were added 1H-pyrrolo[1,2-b]pyridazin-4-one (300.00 mg, 2.237 mmol, 1.00 equiv) and POCl₃ (3.00 mL) at room temperature. The resulting mixture was stirred for 16 h at 80° C. under air atmosphere. The reaction was monitored by LCMS. The reaction was quenched with NaHCO₃ at 0° C. The resulting mixture was extracted with EA (3×30 mL). The combined organic layers were washed with brine (1×40 mL), dried over anhydrous Na₂SO₄. After filtration, the filtrate was concentrated under reduced pressure. The residue was purified by silica gel column chromatography, eluted with PE/MBTE (10:1) to afford 4-chloropyrrolo[1,2-b]pyridazine (130 mg, 38.10%) as a colorless oil. ¹H NMR (400 MHz, Chloroform-d) δ 7.91 (d, J=4.9 Hz, 11H), 7.79 (dd, J=2.8, 1.6 Hz, 11H), 6.89 (dd, J=4.4, 2.7 Hz, 11H), 6.70 (dd, J=4.4, 1.6 Hz, 1H), 6.61 (d, J=4.9 Hz, 1H).

### Step 2

To a stirred solution of 4-chloropyrrolo[1,2-b]pyridazine (100.0 mg, 0.66 mmol, 1.00 equiv) and 3-(4-fluorophenyl)-1-methyl-4-(4,4,5,5-tetramethyl-1,3,2-dioxaborolan-2-yl)pyrazole (297.1 mg, 0.98 mmol, 1.50 equiv) in DMF (0.50 mL) were added Cs₂CO₃ (640.6 mg, 1.97 mmol, 3 equiv) and Pd(dppf)Cl₂CH₂Cl₂ (53.4 mg, 0.07 mmol, 0.1 equiv) in portions at room temperature under nitrogen atmosphere. The resulting mixture was stirred for 2 h at 90° C. under nitrogen atmosphere. The reaction was monitored by LCMS. The residue was purified by reverse flash chromatography with the following conditions: column, C18 silica gel; mobile phase, ACN in water, 10% to 80% gradient in 25 min; detector, UV 254 nm to afford 3-(4-fluorophenyl)-1-methyl-4-[pyrrolo[1,2-b]pyridazin-4-yl]pyrazole (60.5 mg, 35.09%) as a brown solid. ¹H NMR (300 MHz, Chloroform-d) δ 7.93 (d, J=4.6 Hz, 1H), 7.85-7.77 (m, 2H), 7.56-7.43 (m, 2H), 7.09-6.95 (m, 2H), 6.85 (dd, J=4.3, 2.7 Hz, 1H), 6.47 (dd, J=4.3, 1.6 Hz, 1H), 6.28 (d, J=4.7 Hz, 1H), 4.06 (s, 3H). LC/MS (ESI, m/z): [(M+1)]⁺=293.2.

### Example 1-10 Preparation of Compound I-10

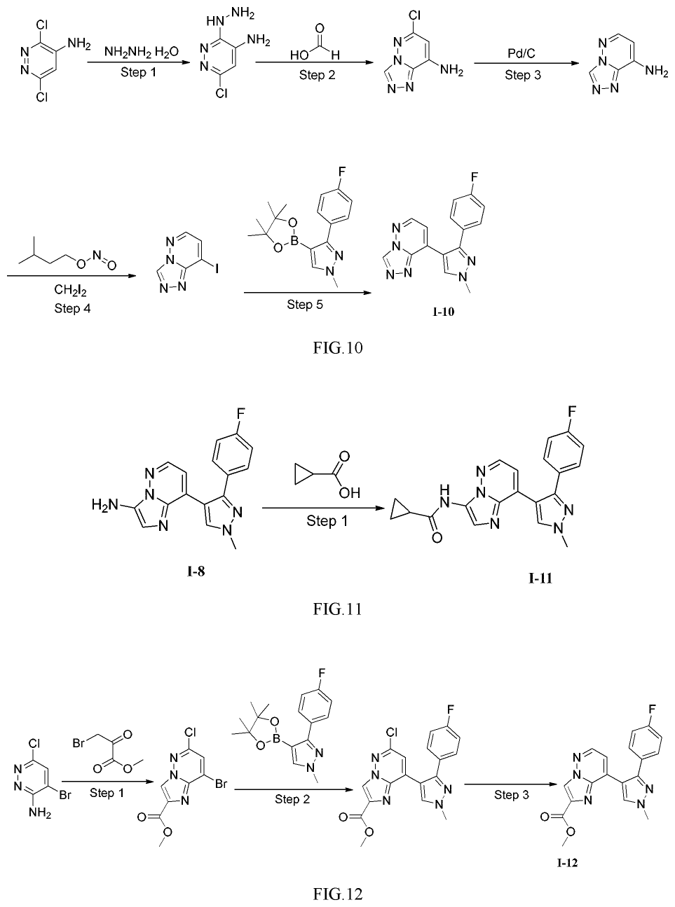

FIG. 10 illustrates the synthetic scheme of compound I-10. As shown in FIG. 10, the specific synthesis steps are as follows:

### Step 1: 6-chloro-3-hydrazinylpyridazin-4-amine

A mixture of 3,6-dichloropyridazin-4-amine (5 g, 30 mmol, 1.0 equiv) and hydrazine hydrate (22 mL, 444 mmol, 15 equiv) in H₂O (25 mL) was stirred for 1 h at 105° C. under nitrogen atmosphere. The mixture was allowed to cool down to room temperature. The resulting mixture was diluted with ice-water (20 mL). The precipitated solids were collected by filtration and washed with ice-water (3×5 mL). The residue was concentrated under vacuum to afford 6-chloro-3-hydrazinylpyridazin-4-amine (3.3 g, 68%) as an off-white solid. ¹H NMR (300 MHz, DMSO-d6) δ 7.29 (s, 1H), 6.38 (s, 1H), 6.26 (br, 2H), 4.24 (br, 2H). LC/MS (ESI, m/z): [(M+1)]⁺=160.

### Step 2: 6-chloro-[1,2,4]triazolo[4,3-b]pyridazin-8-amine

A solution of 6-chloro-3-hydrazinylpyridazin-4-amine (3.3 g, 21 mmol, 1.0 equiv) in formic acid (10 mL) was stirred for 1 h at 110° C. under nitrogen atmosphere. The mixture was allowed to cool down to room temperature. The mixture was neutralized to pH 7 with saturated NaHCO₃ (aq.). The resulting mixture was extracted with EA (3×100 mL). The combined organic layers were washed with brine (2×200 mL), dried over anhydrous Na₂SO₄. After filtration, the filtrate was concentrated under reduced pressure. The residue was purified by silica gel column chromatography, eluted with DCM/MeOH (10:1) to afford 6-chloro-[1,2,4]triazolo[4,3-b]pyridazin-8-amine (2.2 g, 63%) as an off-white solid. ¹H NMR (300 MHz, DMSO-d6) δ 9.39 (s, 1H), 7.95 (br, 2H), 6.13 (s, 1H). LC/MS (ESI, m/z): [(M+1)]⁺=170.

### Step 3: [1,2,4]triazolo[4,3-b]pyridazin-8-amine

To a solution of 6-chloro-[1,2,4]triazolo[4,3-b]pyridazin-8-amine (2.2 g, 13 mmol, 1.0 equiv) and DIEA (2.3 mL, 13 mmol, 1.0 equiv) in EtOH (70 mL) was added Pd/C (550 mg, 10%) under nitrogen atmosphere. The mixture was hydrogenated at 50° C. for 16 h under hydrogen atmosphere using a hydrogen tyre. The resulting mixture was filtered and the filter cake was washed with MeOH (3×10 mL). The filtrate was concentrated under reduced pressure to afford [1,2,4]triazolo[4,3-b]pyridazin-8-amine (3.8 g, crude) as a grey semi-solid. ¹H NMR (400 MHz, DMSO-d6) δ 9.38 (s, 1H), 8.04 (d, J=5.4 Hz, 1H), 7.48 (br, 2H), 6.09 (d, J=5.5 Hz, 1H). LC/MS (ESI, m/z): [(M+1)]⁺=136.

### Step 4: 8-iodo-[1,2,4]triazolo[4,3-b]pyridazine

To a stirred solution of [1,2,4]triazolo[4,3-b]pyridazin-8-amine (1.6 g, 12 mmol, 1.0 equiv) and CH₂I₂ (10 mL) in ACN (100 mL) was added isoamyl nitrite (6.9 g, 59 mmol, 5.0 equiv) dropwise at room temperature under nitrogen atmosphere. The resulting mixture was stirred for 1 h at 80° C. under nitrogen atmosphere. The resulting mixture was concentrated under reduced pressure. The resulting mixture was diluted with water (50 mL). The resulting mixture was extracted with DCM (3×80 mL). The combined organic layers were washed with brine (2×200 mL), dried over anhydrous Na₂SO₄. After filtration, the filtrate was concentrated under reduced pressure. The residue was purified by silica gel column chromatography, eluted with DCM/MeOH (10:1) to afford 8-iodo-[1,2,4]triazolo[4,3-b]pyridazine (410 mg, 14%) as a brown solid. ¹H NMR (400 MHz, DMSO-d6) δ 9.80 (s, 1H), 8.23 (d, J=4.5 Hz, 1H), 7.94 (d, J=4.5 Hz, 1H). LC/MS (ESI, m/z): [(M+1)]⁺=247.

### Step 5

To a stirred mixture of 8-iodo-[1,2,4]triazolo[4,3-b]pyridazine (100 mg, 0.41 mmol, 1.0 equiv) and 3-(4-fluorophenyl)-1-methyl-4-(4,4,5,5-tetramethyl-1,3,2-dioxaborolan-2-yl)pyrazole (147 mg, 0.49 mmol, 1.2 equiv) in DMF (2.5 mL) were added Cs₂CO₃ (266 mg, 0.81 mmol, 2.0 equiv) and Pd(dppf)Cl₂CH₂Cl₂ (33 mg, 0.04 mmol, 0.1 equiv) at room temperature under nitrogen atmosphere. The resulting mixture was stirred for 2 h at 80° C. under nitrogen atmosphere. The resulting mixture was concentrated under reduced pressure. The residue was purified by Prep-HPLC with the following conditions (Column: X Select CSH Prep C18 OBD Column, 19*250 mm, 5 um; Mobile Phase A: water (0.05% FA), Mobile Phase B: ACN; Flow rate: 25 mL/min; Gradient: 20 B to 45 B in 7 min, 254/220 nm; RT₁: 5.93 min; Injection Volume: 0.4 ml; Number Of Runs: 8) to afford 3-(4-fluorophenyl)-1-methyl-4-[[1,2,4]triazolo[4,3-b]pyridazin-8-yl]pyrazole (37.8 mg, 32%) as a pink solid. ¹H NMR (300 MHz, CD₃OD) δ 9.45 (s, 1H), 8.79 (s, 1H), 8.29 (d, J=4.8 Hz, 1H), 7.59-7.52 (m, 2H), 7.25-7.17 (m, 2H), 6.87 (d, J=4.8 Hz, 1H), 4.05 (s, 3H). LC/MS (ESI, m/z): [(M+1)]⁺=295.

### Example 1-11 Preparation of Compound I-11

FIG. 11 illustrates the synthetic scheme of compound I-11. As shown in FIG. 11, the specific synthesis steps are as follows:

### Step 1

A solution of cyclopropanecarboxylic acid (56 mg, 0.65 mmol, 2.0 equiv) and HATU (185 mg, 0.49 mmol, 1.5 equiv) in DMF (2 mL) was stirred for 15 min at room temperature under nitrogen atmosphere. To the above mixture was added 8-[3-(4-fluorophenyl)-1-methylpyrazol-4-yl]imidazo[1,2-b]pyridazin-3-amine (100 mg, 0.32 mmol, 1.0 equiv) and DIPEA (105 mg, 0.81 mmol, 2.5 equiv) at room temperature. The resulting mixture was stirred for additional 16 h at 50° C. The mixture was purified by Prep-HPLC with the following conditions (Column: X Select CSH Prep C18 OBD Column, 19*250 mm, 5 um; Mobile Phase A: water (0.05% FA), Mobile Phase B: ACN; Flow rate: 25 mL/min; Gradient: 15 B to 39 B in 7 min, 254/220 nm; RT₁: 6.17 min; Injection Volume: 0.4 ml; Number Of Runs: 8) to afford N-[8-[3-(4-fluorophenyl)-1-methylpyrazol-4-yl]imidazo[1,2-b]pyridazin-3-yl]cyclopropanecarboxamide (26.4 mg, 22%) as a light yellow solid. ¹H NMR (400 MHz, CDCl₃) δ 8.78 (s, 1H), 8.54 (s, 11H), 8.07 (d, J=4.9 Hz, 11H), 7.98 (d, J=1.2 Hz, 11H), 7.77 (d, J=1.3 Hz, 11H), 7.56 (s, 11H), 7.22-7.17 (m, 1H), 7.16-7.09 (m, 1H), 6.84 (d, J=4.9 Hz, 1H), 4.01 (s, 3H), 1.59-1.52 (m, 1H), 1.10-1.04 (m, 2H), 0.90-0.83 (m, 2H). LC/MS (ESI, m/z): [(M+1)]⁺=377.

### Example 1-12 Preparation of Compound I-12

FIG. 12 illustrates the synthetic scheme of compound I-12. As shown in FIG. 12, the specific synthesis steps are as follows:

### Step 1: methyl 8-bromo-6-chloroimidazo[1,2-b]pyridazine-2-carboxylate

To a stirred mixture of 4-bromo-6-chloropyridazin-3-amine (5 g, 24 mmol, 1.0 equiv) and methyl 3-bromo-2-oxopropanoate (17 g, 96 mmol, 4.0 equiv) in DME (50 mL) at room temperature under air atmosphere. The reaction stirred for 16 h at 90° C. The resulting mixture was concentrated under reduced pressure. The residue was purified by silica gel column chromatography, eluted with PE/EA (3:1) to afford methyl 8-bromo-6-chloroimidazo[1,2-b]pyridazine-2-carboxylate (5.7 g, 81%) as an off-white solid. ¹H NMR (300 MHz, CDCl₃) δ 8.50 (s, 1H), 7.47 (s, 1H), 4.00 (s, 3H). LC/MS (ESI, m/z): [(M+1)]⁺=290, 292.

### Step 2: 6-chloro-8-[3-(4-fluorophenyl)-1-methylpyrazol-4-yl]imidazo[1,2-b]pyridazine-2-carboxylate

To a stirred mixture of methyl 8-bromo-6-chloroimidazo[1,2-b]pyridazine-2-carboxylate (1 g, 3.4 mmol, 1.0 equiv) and 3-(4-fluorophenyl)-1-methyl-4-(4,4,5,5-tetramethyl-1,3,2-dioxaborolan-2-yl)pyrazole (1 g, 3.4 mmol, 1.0 equiv) in toluene (15 mL) were added Cs₂CO₃ (2.3 g, 6.9 mmol, 2.0 equiv) and Pd(dppf)Cl₂CH₂Cl₂ (280 mg, 0.34 mmol, 0.1 equiv) at room temperature under nitrogen atmosphere. The resulting mixture was stirred for 2 h at 100° C. under nitrogen atmosphere. The resulting mixture was concentrated under reduced pressure. The residue was purified by silica gel column chromatography, eluted with PE/EA (1:2) to afford methyl 6-chloro-8-[3-(4-fluorophenyl)-1-methylpyrazol-4-yl]imidazo[1,2-b]pyridazine-2-carboxylate (390 mg, 29%) as a light brown solid. ¹H NMR (400 MHz, CDCl₃) δ 8.87 (s, 1H), 8.43 (d, J=0.8 Hz, 1H), 7.51 (dd, J=8.6, 5.6 Hz, 2H), 7.16 (t, J=8.6 Hz, 2H), 6.79 (s, 1H), 4.05 (s, 3H), 4.01 (s, 3H). LC/MS (ESI, m/z): [(M+1)]⁺=386.

### Step 3

To a solution of methyl 6-chloro-8-[3-(4-fluorophenyl)-1-methylpyrazol-4-yl]imidazo[1,2-b]pyridazine-2-carboxylate (390 mg, 1.0 mmol, 1.0 equiv) in EA (10 mL) was added Pd/C (97 mg, 10%) under nitrogen atmosphere. The mixture was hydrogenated at 50° C. for 16 h under hydrogen atmosphere using a hydrogen tyre. The resulting mixture was filtered and the filter cake was washed with MeOH (3×10 mL). The filtrate was concentrated under reduced pressure. The residue was purified by Prep-HPLC with the following conditions (Column: X Select CSH Prep C18 OBD Column, 19*250 mm, 5 um; Mobile Phase A: water (0.05% FA), Mobile Phase B: ACN; Flow rate: 25 mL/min; Gradient: 30 B to 55 B in 7 min, 254/220 nm; RT₁: 6.47 min; Injection Volume: 0.4 ml; Number Of Runs: 8) to afford methyl 8-[3-(4-fluorophenyl)-1-methylpyrazol-4-yl]imidazo[1,2-b]pyridazine-2-carboxylate (29.5 mg, 8%) as an off-white solid. ¹H NMR (400 MHz, CDCl₃) δ 8.84 (s, 1H), 8.52 (s, 1H), 8.12 (d, J=4.9 Hz, 1H), 7.52-7.47 (m, 2H), 7.15-7.09 (m, 2H), 6.77 (d, J=5.0 Hz, 1H), 4.04 (s, 3H), 4.01 (s, 3H). LC/MS (ESI, m/z): [(M+1)]⁺=352.

### Example 1-13 Preparation of Compound I-13

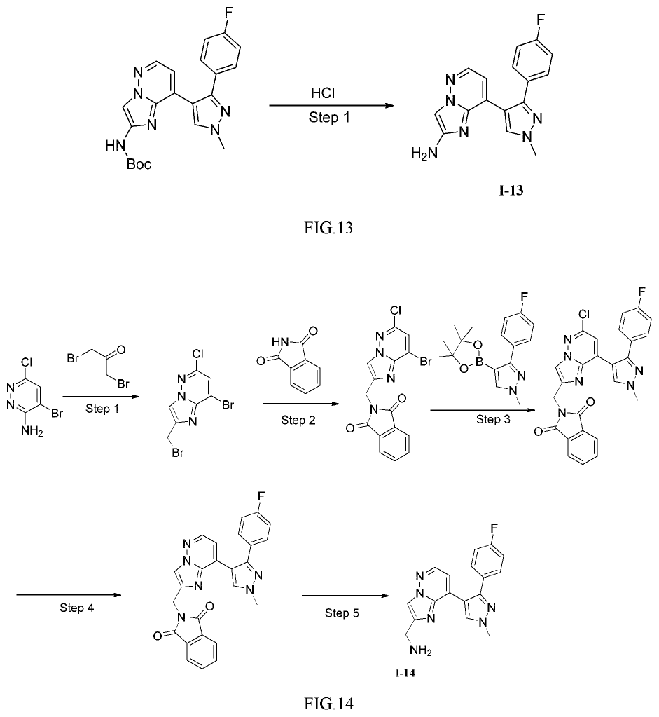

FIG. 13 illustrates the synthetic scheme of compound 1-13. As shown in FIG. 13, the specific synthesis steps are as follows:

### Step 1

A solution of tert-butyl N-[8-[3-(4-fluorophenyl)-1-methylpyrazol-4-yl]imidazo[1,2-b]pyridazin-2-yl]carbamate (40 mg, 0.1 mmol, 1.0 equiv) in HCl (5 mL, 4 M in HCl) was stirred for 1 h at room temperature under nitrogen atmosphere. The resulting mixture was concentrated under reduced pressure. The residue was purified by Prep-HPLC with the following conditions (Column: X Select CSH Prep C18 OBD Column, 19*250 mm, 5 um; Mobile Phase A: water (0.05% FA), Mobile Phase B: ACN; Flow rate: 25 mL/min; Gradient: 12 B to 38 B in 7 min, 254/220 nm; RT1:6.72 min; Injection Volume: 0.4 ml; Number Of Runs: 8) to afford 8-[3-(4-fluorophenyl)-1-methylpyrazol-4-yl]imidazo[1,2-b]pyridazin-2-amine; formic acid (17.1 mg, 49%) as a yellow solid. ¹H NMR (400 MHz, CDCl₃) δ 8.56-8.52 (m, 1H), 7.92 (td, J=3.0, 1.5 Hz, 1H), 7.53-7.46 (m, 2H), 7.36 (q, J=1.5 Hz, 1H), 7.12-7.05 (m, 2H), 6.61-6.57 (m, 1H), 4.02 (s, 3H). LC/MS (ESI, m/z): [(M+1−FA)]⁺=309.

### Example 1-14 Preparation of Compound I-14

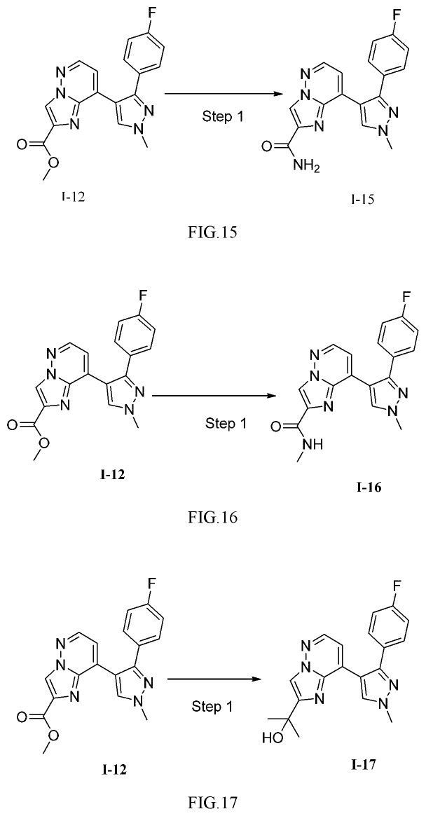

FIG. 14 illustrates the synthetic scheme of compound 1-14. As shown in FIG. 14, the specific synthesis steps are as follows:

### Step 1: 8-bromo-2-(bromomethyl)-6-chloroimidazo[1,2-b]pyridazine

To a stirred mixture of 4-bromo-6-chloropyridazin-3-amine (12.05 g, 57.810 mmol, 1.00 equiv) in DME (100.00 mL) were added 2-propanone, 1,3-dibromo-(24.96 g, 115.624 mmol, 2.00 equiv) at room temperature under air atmosphere. The solution was stirred at 90° C. for 2 h. The residue was purified by silica gel column chromatography, eluted with PE/EA (5:1) to afford 8-bromo-2-(bromomethyl)-6-chloroimidazo[1,2-b]pyridazine(7.4 g, 39.34%) as a white solid. LC/MS (ESI, m/z): [(M+1)]⁺=324.1.

### Step 2: 2-([8-bromo-6-chloroimidazo[1,2-b]pyridazin-2-yl]methyl)isoindole-1,3-dione

To a stirred solution of 8-bromo-2-(bromomethyl)-6-chloroimidazo[1,2-b]pyridazine(13.30 g, 40.874 mmol, 1.00 equiv) and phthalimide(6.62 g, 0.045 mmol, 1.10 equiv) in dioxane(200.00 mL) was added K₂CO₃ (11.30 g, 81.748 mmol, 2.00 equiv) at room temperature under air atmosphere. The mixture was stirred for 1 h at 80° C., the reaction was monitored by LCMS. The reaction mixture was concentrated under reduced pressure. The residue was purified by reverse flash chromatography with the following conditions: column, C18 silica gel; mobile phase, ACN in 0.1% FA, 30% to 50% gradient in 20 min; detector, UV 254 nm. to afford 2-([8-bromo-6-chloroimidazo[1,2-b]pyridazin-2-yl]methyl)isoindole-1,3-dione (10 g, 62.47%) as a white solid. LC/MS (ESI, m/z): [(M+1)]⁺=393.1.

### Step 3: 2-([6-chloro-8-[3-(4-fluorophenyl)-1-methylpyrazol-4-yl]imidazo[1,2-b]pyridazin-2-yl]methyl)isoindole-1,3-dione

To a stirred solution of 2-([8-bromo-6-chloroimidazo[1,2-b]pyridazin-2-yl]methyl)isoindole-1,3-dione(5.70 g, 14.555 mmol, 1.00 equiv) and 3-(4-fluorophenyl)-1-methyl-4-(4,4,5,5-tetramethyl-1,3,2-dioxaborolan-2-yl)pyrazole(6.60 g, 0.023 mmol, 1.50 equiv) in Toluene(90.00 mL) were added Pd(dppf)Cl₂·CH₂Cl₂ (1.19 g, 1.456 mmol, 0.10 equiv) and Cs₂CO₃ (14.23 g, 43.665 mmol, 3.00 equiv) at room temperature under nitrogen atmosphere. The resulting mixture was stirred for 2 h at 100° C. under nitrogen atmosphere. The reaction was monitored by LCMS. The residue was purified by reverse flash chromatography with the following conditions: column, C18 silica gel; mobile phase, ACN in 0.1% FA, 10% to 50% gradient in 10 min; detector, UV 254 nm to afford 2-([6-chloro-8-[3-(4-fluorophenyl)-1-methylpyrazol-4-yl]imidazo[1,2-b]pyridazin-2-yl]methyl)isoindole-1,3-dione(2 g, 28.22%) as a yellow solid. LC/MS (ESI, m/z): [(M+1)]⁺=487.3.

### Step 4: 2-([8-[3-(4-fluorophenyl)-1-methylpyrazol-4-yl]imidazo[1,2-b]pyridazin-2-yl]methyl)isoindole-1,3-dione

To a stirred solution of 2-([6-chloro-8-[3-(4-fluorophenyl)-1-methylpyrazol-4-yl]imidazo[1,2-b]pyridazin-2-yl]methyl)isoindole-1,3-dione (2.00 g, 4.108 mmol, 1.00 equiv) in EtOH (60.00 mL) was added Pd/C (437.14 mg, 0.411 mmol, 0.10 equiv, 10%). The resulting mixture was stirred for 48 h at 50° C. under hydrogen atmosphere. The reaction was monitored by LCMS. The resulting mixture was filtered, the filter cake was washed with DCM (3×30 mL). The filtrate was concentrated under reduced pressure. This resulted in 2-([8-[3-(4-fluorophenyl)-1-methylpyrazol-4-yl]imidazo[1,2-b]pyridazin-2-yl]methyl)isoindole-1,3-dione (1 g, 53.81%) as a yellow solid. The crude product was used to the next step directly without further purification. LC/MS (ESI, m/z): [(M+1)]⁺=453.1.

### Step 5

To a stirred solution of 2-([8-[3-(4-fluorophenyl)-1-methylpyrazol-4-yl]imidazo[1,2-b]pyridazin-2-yl]methyl)isoindole-1,3-dione (100.00 mg, 0.221 mmol, 1.00 equiv) in EtOH (1.00 mL) was added N₂H₄·H₂O (66.39 mg, 1.326 mmol, 6 equiv). The resulting mixture was stirred for 1 h at 80° C. under air atmosphere. The reaction was monitored by LCMS. The residue was purified by reverse flash chromatography with the following conditions: column, C18 silica gel; mobile phase, ACN in 0.1% FA aq., 10% to 50% gradient in 25 min; detector, UV 254 nm to afford 1-[8-[3-(4-fluorophenyl)-1-methylpyrazol-4-yl]imidazo[1,2-b]pyridazin-2-yl]methanamine (13.4 mg, 18.81%) as an off-white solid. ¹H NMR (300 MHz, Methanol-d4) δ 8.88 (s, 1H), 8.58 (s, 1H), 8.26-8.17 (m, 2H), 7.59-7.47 (m, 2H), 7.28-7.14 (m, 2H), 6.84 (d, J=4.9 Hz, 1H), 4.35 (d, J=0.7 Hz, 2H), 4.06 (s, 3H). LC/MS (ESI, m/z): [(M+1)]⁺=323.15.

### Example 1-15 Preparation of Compound I-15

FIG. 15 illustrates the synthetic scheme of compound I-15. As shown in FIG. 15, the specific synthesis steps are as follows:

### Step 1

Into a 40 mL sealed tube were added methyl 8-[3-(4-fluorophenyl)-1-methylpyrazol-4-yl]imidazo[1,2-b]pyridazine-2-carboxylate (100.00 mg, 0.285 mmol, 1.00 equiv) and NH₃·H₂O (5.00 mL), the reaction was allowed to reacted for 16 h at 80° C., then cooled down to room temperature. The solution was concentrated under reduced pressure and the residue purified by reverse flash chromatography with the following conditions: column, C18 silica gel; mobile phase, ACN in 0.1% FA aq., 30% to 50% gradient in 15 min; detector, UV 254 nm. This resulted in 8-[3-(4-fluorophenyl)-1-methylpyrazol-4-yl]imidazo[1,2-b]pyridazine-2-carboxamide (15.4 mg, 16.09%) as a yellow solid. ¹H NMR (300 MHz, Chloroform-d) δ 8.68 (s, 1H), 8.55 (s, 1H), 8.15 (d, J=4.9 Hz, 1H), 7.59-7.47 (m, 2H), 7.23 (s, 1H), 7.21-7.09 (m, 2H), 6.81 (d, J=4.9 Hz, 1H), 5.60 (s, 1H), 4.09 (s, 3H). LC/MS (ESI, m/z): [(M+1)]⁺=337.1.

### Example 1-16 Preparation of Compound I-16

FIG. 16 illustrates the synthetic scheme of compound I-16. As shown in FIG. 16, the specific synthesis steps are as follows:

### Step 1

Into a 40 mL sealed tube were added methyl 8-[3-(4-fluorophenyl)-1-methylpyrazol-4-yl]imidazo[1,2-b]pyridazine-2-carboxylate (100.00 mg, 0.285 mmol, 1.00 equiv) and methylamine in THF (5 mL, 2M), the solution was allowed to reacted for 16 h at 100° C. The reaction was cooled down to room temperature, then concentrated under reduced pressure. The residue was purified by reverse flash chromatography with the following conditions: column, C18 silica gel; mobile phase, ACN in 0.1% FA aq., 40% to 60% gradient in 15 min; detector, UV 254 nm. This resulted in 8-[3-(4-fluorophenyl)-1-methylpyrazol-4-yl]-N-methylimidazo[1,2-b]pyridazine-2-carboxamide(25.7 mg, 25.77%) as an off-white solid. 1H NMR (300 MHz, Chloroform-d) δ 8.65 (s, 1H), 8.53 (s, 1H), 8.15 (d, J=4.9 Hz, 1H), 7.58-7.47 (m, 2H), 7.36 (s, 1H), 7.15 (t, J=8.7 Hz, 2H), 6.80 (d, J=4.9 Hz, 1H), 4.10 (s, 3H), 3.11 (d, J=5.1 Hz, 3H). LC/MS (ESI, m/z): [(M+1)]⁺=351.2

### Example 1-17 Preparation of Compound I-17

FIG. 17 illustrates the synthetic scheme of compound I-17. As shown in FIG. 17, the specific synthesis steps are as follows:

### Step 1

To a stirred mixture of methyl 8-[3-(4-fluorophenyl)-1-methylpyrazol-4-yl]imidazo[1,2-b]pyridazine-2-carboxylate (100 mg, 0.285 mmol, 1.00 equiv) in THF (10.00 mL) was added MeMgBr (0.60 mL, 1.800 mmol, 6.32 equiv) dropwise at 0° C. under air atmosphere. The reaction was reacted for 1 hour at room temperature. The reaction was monitored by LCMS, then quenched by NHCl₄ aq., concentrated under reduced pressure. The residue was purified by reverse flash chromatography with the following conditions: column, C18 silica gel; mobile phase, ACN in 0.1% FA aq., 35% to 55% gradient in 15 min; detector, UV 254 nm. This resulted in 2-[8-[3-(4-fluorophenyl)-1-methylpyrazol-4-yl]imidazo[1,2-b]pyridazin-2-yl]propan-2-ol (29.5 mg, 29.50%) as a light yellow solid. H NMR (300 MHz, Chloroform-d) δ 8.80 (s, 1H), 8.08 (s, 1H), 7.89 (s, 1H), 7.53 (dd, J=8.6, 5.4 Hz, 2H), 7.14 (t, J=8.7 Hz, 2H), 6.76 (s, 1H), 4.08 (s, 3H), 1.74 (s, 6H). LC/MS (ESI, m/z): [(M+1)]⁺=352.2.

### Example 1-18 Preparation of Compound I-18

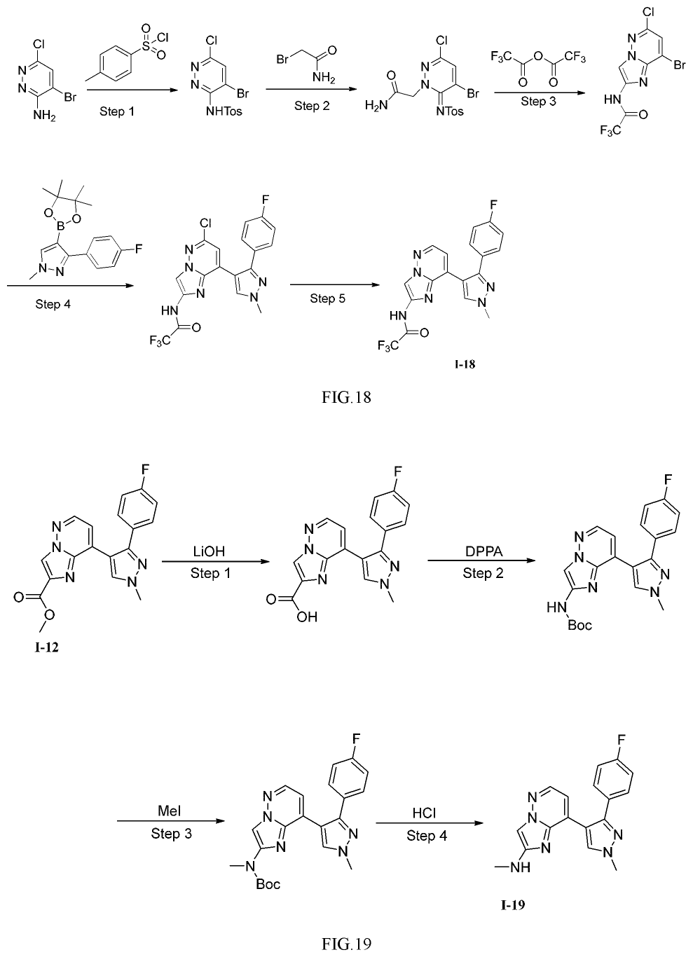

FIG. 18 illustrates the synthetic scheme of compound I-18. As shown in FIG. 18, the specific synthesis steps are as follows:

### Step 1: N-(4-bromo-6-chloropyridazin-3-yl)-4-methylbenzenesulfonamide

To a stirred mixture of 4-bromo-6-chloropyridazin-3-amine (10.00 g, 47.975 mmol, 1.00 equiv) in THF (100.00 mL) was added NaH (3.84 g, 96.009 mmol, 2.00 equiv, 60%) in portions at 0° C. under N₂ atmosphere. The resulting mixture was stirred for 15 min at room temperature under nitrogen atmosphere. To the above mixture was added P-toluenesulfonyl chloride (10.98 g, 57.570 mmol, 1.20 equiv) in THF (100.00 mL) in portions at 0° C. under N₂ atmosphere. The resulting mixture was stirred for 2 h at RT under N₂ atmosphere. Desired product could be detected by LCMS. The reaction was quenched with H₂O (20 ml) at RT. The resulting mixture was concentrated under reduced pressure. The residue was purified by silica gel column chromatography, eluted with DCM/MeOH (1:10) to afford N-(4-bromo-6-chloropyridazin-3-yl)-4-methylbenzenesulfonamide (9.1 g, 52.31%) as a light yellow solid. ¹H NMR (300 MHz, Chloroform-d) δ 7.97 (s, 2H), 7.66 (s, 1H), 7.29 (s, 2H), 2.41 (s, 3H). LC/MS (ESI, m/z): [(M+1)]⁺=362.0, 364.0.

### Step 2: 2-[(6Z)-5-bromo-3-chloro-6-[(4-methylbenzenesulfonyl)imino]pyridazin-1-yl]acetamide

To a stirred mixture of N-(4-bromo-6-chloropyridazin-3-yl)-4-methylbenzenesulfonamide (100.00 mg, 0.276 mmol, 1.00 equiv), DIEA (891.01 mg, 6.895 mmol, 5.00 equiv) and bromoacetamide (190.23 mg, 1.380 mmol, 5.00 equiv) in DMF (5.00 mL) at RT under N₂ atmosphere. The resulting mixture was stirred for 4 h at 80° C. under N₂ atmosphere. The resulting mixture was concentrated under reduced pressure. The residue was purified by silica gel column chromatography, eluted with DCM/MeOH (1:10) to afford crude, The crude was purified by reverse flash chromatography with the following conditions: column, C18 silica gel; detector, UV 254 nm/220 nm; mobile phase ACN in water (0.1% mol/L FA), 20% to 50% gradient in 30 min to afford 2-[(6Z)-5-bromo-3-chloro-6-[(4-methylbenzenesulfonyl)imino]pyridazin-1-yl]acetamide (100 mg, 17.28%) as a yellow solid. LC/MS (ESI, m/z): [(M+1)]⁺=419.1, 421.1.

### Step 3: N-[8-bromo-6-chloroimidazo[1,2-b]pyridazin-2-yl]-2,2,2-trifluoroacetamide

To a stirred mixture of 2-[(6Z)-5-bromo-3-chloro-6-[(4-methylbenzenesulfonyl)imino]pyridazin-1-yl]acetamide (420.00 mg, 1.001 mmol, 1.00 equiv) in DCM (8.00 mL) was added trifluoroacetic anhydride (2.1 mL) in portions at 0° C. The resulting mixture was stirred for 1 h at room temperature. Desired product could be detected by LCMS. The resulting mixture was concentrated under reduced pressure. The residue was purified by silica gel column chromatography, eluted with DCM/MeOH (1:10) to afford N-[8-bromo-6-chloroimidazo[1,2-b]pyridazin-2-yl]-2,2,2-trifluoroacetamide (285 mg, 82.91%) as a yellow oil. LC/MS (ESI, m/z): [(M+1)]⁺=342.9, 344.9.

### Step 4: N-[6-chloro-8-[3-(4-fluorophenyl)-1-methylpyrazol-4-yl]imidazo[1,2-b]pyridazin-2-yl]-2,2,2-trifluoroacetamide

To a stirred mixture of N-[8-bromo-6-chloroimidazo[1,2-b]pyridazin-2-yl]-2,2,2-trifluoroacetamide (500.00 mg, 1.456 mmol, 1.00 equiv), 3-(4-fluorophenyl)-1-methyl-4-(4,4,5,5-tetramethyl-1,3,2-dioxaborolan-2-yl)pyrazole (439.84 mg, 1.456 mmol, 1.00 equiv), Pd(dppf)Cl₂CH₂Cl₂ (118.58 mg, 0.146 mmol, 0.10 equiv) and Cs₂CO₃ (948.56 mg, 2.911 mmol, 2.00 equiv) in DMF (15.00 mL) at RT under N₂ atmosphere. The resulting mixture was stirred for 12 h at 90° C. under N₂ atmosphere. Desired product could be detected by LCMS. The resulting mixture was purified by reverse flash chromatography with the following conditions: column, C18 silica gel; detector, UV 254 nm/220 nm; mobile phase ACN in water (0.1% mol/L FA), 40% to 80% gradient in 40 min to afford crude. The crude was purified by silica gel column chromatography, eluted with DCM/MeOH (1:10) to afford N-[6-chloro-8-[3-(4-fluorophenyl)-1-methylpyrazol-4-yl]imidazo[1,2-b]pyridazin-2-yl]-2,2,2-trifluoroacetamide (53 mg, 8.30%) as a yellow solid. LC/MS (ESI, m/z): [(M+1)]⁺=439.2.

### Step 5

To a stirred mixture of N-[6-chloro-8-[3-(4-fluorophenyl)-1-methylpyrazol-4-yl]imidazo[1,2-b]pyridazin-2-yl]-2,2,2-trifluoroacetamide (53.00 mg, 0.121 mmol, 1.00 equiv) in MeOH (2.00 mL) and EA (2.00 mL) was added Pd/C (12.85 mg, 0.012 mmol, 0.10 equiv, 10%) at RT under H₂ atmosphere. The resulting mixture was stirred for 12 h at 50° C. under H₂ atmosphere. Desired product could be detected by LCMS. The resulting mixture was filtered, the filtrate was concentrated under reduced pressure. The residue was purified by reverse flash chromatography with the following conditions: column, C18 silica gel; detector, UV 254 nm/220 nm; mobile phase ACN in water (0.1% mol/L FA), 30% to 60% gradient in 30 min to afford 2,2,2-trifluoro-N-[8-[3-(4-fluorophenyl)-1-methylpyrazol-4-yl]imidazo[1,2-b]pyridazin-2-yl]acet amide (15 mg, 30.71%) as a light yellow solid. 1H NMR (400 MHz, Chloroform-d) δ 8.94 (s, 1H), 8.49 (d, J=4.7 Hz, 1H), 8.30 (s, 1H), 7.83 (dd, J=8.8, 5.4 Hz, 2H), 7.16-7.08 (m, 4H), 4.10 (s, 3H). LC/MS (ESI, m/z): [(M+1)]⁺=405.1.

### Example 1-19 Preparation of Compound I-19

FIG. 19 illustrates the synthetic scheme of compound I-19. As shown in FIG. 19, the specific synthesis steps are as follows:

### Step 1: 8-[3-(4-fluorophenyl)-1-methylpyrazol-4-yl]imidazo[1,2-b]pyridazine-2-carboxylic acid

To a stirred solution of methyl 8-[3-(4-fluorophenyl)-1-methylpyrazol-4-yl]imidazo[1,2-b]pyridazine-2-carboxylate (970 mg, 2.8 mmol, 1.0 equiv) in MeOH (7 mL)/THF (7 mL)/H₂O (7 mL) was added LiOH·H₂O (348 mg, 8.3 mmol, 3.0 equiv) at room temperature under nitrogen atmosphere. The resulting mixture was stirred for 2 h at room temperature under nitrogen atmosphere. The resulting mixture was concentrated under reduced pressure. The residue was acidified to pH 4 with 3 N HCl (aq.). The resulting mixture was extracted with CHCl₃/IPA (3×80 mL). The combined organic layers were washed with brine (2×200 mL), dried over anhydrous Na₂SO₄. After filtration, the filtrate was concentrated under reduced pressure. The residue was purified by silica gel column chromatography, eluted with DCM/MeOH (10:1) to afford 8-[3-(4-fluorophenyl)-1-methylpyrazol-4-yl]imidazo[1,2-b]pyridazine-2-carboxylic acid (280 mg, 30%) as a light brown solid. LC/MS (ESI, m/z): [(M+1)]⁺=338.

### Step 2: tert-butyl N-[8-[3-(4-fluorophenyl)-1-methylpyrazol-4-yl]imidazo[1,2-b]pyridazin-2-yl]carbamate

To a stirred solution of 8-[3-(4-fluorophenyl)-1-methylpyrazol-4-yl]imidazo[1,2-b]pyridazine-2-carboxylic acid (270 mg, 0.8 mmol, 1.0 equiv) and Et₃N (162 mg, 1.6 mmol, 2.0 equiv) in t-BuOH (15 mL) was added DPPA (330 mg, 1.2 mmol, 1.5 equiv) at room temperature under nitrogen atmosphere. The resulting mixture was stirred for 10 min at room temperature under nitrogen atmosphere. Then it was stirred for 2 h at 100 degrees C. under nitrogen atmosphere. The resulting mixture was concentrated under reduced pressure. The resulting mixture was diluted with EA (30 mL). The mixture was neutralized to pH 8 with saturated NaHCO₃ (aq.). The resulting mixture was extracted with EA (3×30 mL). The combined organic layers were washed with brine (2×80 mL), dried over anhydrous Na₂SO₄. After filtration, the filtrate was concentrated under reduced pressure. The residue was purified by silica gel column chromatography, eluted with PE/EA (1:1) to afford tert-butyl N-[8-[3-(4-fluorophenyl)-1-methylpyrazol-4-yl]imidazo[1,2-b]pyridazin-2-yl]carbamate (150 mg, 46%) as a light yellow solid. LC/MS (ESI, m/z): [(M+1)]⁺=409.

### Step 3: tert-butyl N-[8-[3-(4-fluorophenyl)-1-methylpyrazol-4-yl]imidazo[1,2-b]pyridazin-2-yl]-N-methylcarbamate

To a stirred solution of tert-butyl N-[8-[3-(4-fluorophenyl)-1-methylpyrazol-4-yl]imidazo[1,2-b]pyridazin-2-yl]carbamate (70 mg, 0.17 mmol, 1.0 equiv) in THF (2 mL) was added NaH (14 mg, 0.34 mmol, 2.0 equiv, 60%) in portions at 0° C. under nitrogen atmosphere. The resulting mixture was stirred for 15 min at 0° C. under nitrogen atmosphere. To the above mixture was added Mel (37 mg, 0.26 mmol, 1.5 equiv) dropwise at 0 degrees C. The resulting mixture was stirred for additional 1 h at room temperature. The reaction was quenched by the addition of sat. NH₄Cl(aq.) (5 mL) at 0° C. The resulting mixture was extracted with EA (3×15 mL). The combined organic layers were washed with brine (2×30 mL), dried over anhydrous Na₂SO₄. After filtration, the filtrate was concentrated under reduced pressure. The residue was purified by silica gel column chromatography, eluted with PE/EA (1:1) to afford tert-butyl N-[8-[3-(4-fluorophenyl)-1-methylpyrazol-4-yl]imidazo[1,2-b]pyridazin-2-yl]-N-methylcarbamate (60 mg, 83%) as a light yellow solid. LC/MS (ESI, m/z): [(M+1)]⁺=423.

### Step 4

A solution of tert-butyl N-[8-[3-(4-fluorophenyl)-1-methylpyrazol-4-yl]imidazo[1,2-b]pyridazin-2-yl]-N-methylcarbamate (60 mg, 0.14 mmol, 1.0 equiv) in HCl (5 mL, 4 M in EA) was stirred for 1 h at room temperature under nitrogen atmosphere. The resulting mixture was concentrated under reduced pressure. The residue was purified by Prep-HPLC with the following conditions (Column: X Select CSH Prep C18 OBD Column, 19*250 mm, 5 um; Mobile Phase A: water (0.05% FA), Mobile Phase B: ACN; Flow rate: 25 mL/min; Gradient: 18 B to 48 B in 7 min, 254/220 nm; RT₁: 6.22 min; Injection Volume: 0.4 ml; Number Of Runs: 8) to afford 8-[3-(4-fluorophenyl)-1-methylpyrazol-4-yl]-N-methylimidazo[1,2-b]pyridazin-2-amine (14.3 mg, 31%) as a yellow solid. ¹H NMR (300 MHz, CDCl₃) δ 8.54 (s, 1H), 7.89 (d, J=5.1 Hz, 1H), 7.54-7.47 (m, 2H), 7.25 (s, 1H), 7.13-7.03 (m, 2H), 6.58 (d, J=5.1 Hz, 1H), 4.01 (s, 3H), 2.94 (s, 311). LC/MS (ESI, m/z): [(M+1)]⁺=323.

### Example 1-20 Preparation of Compound I-20

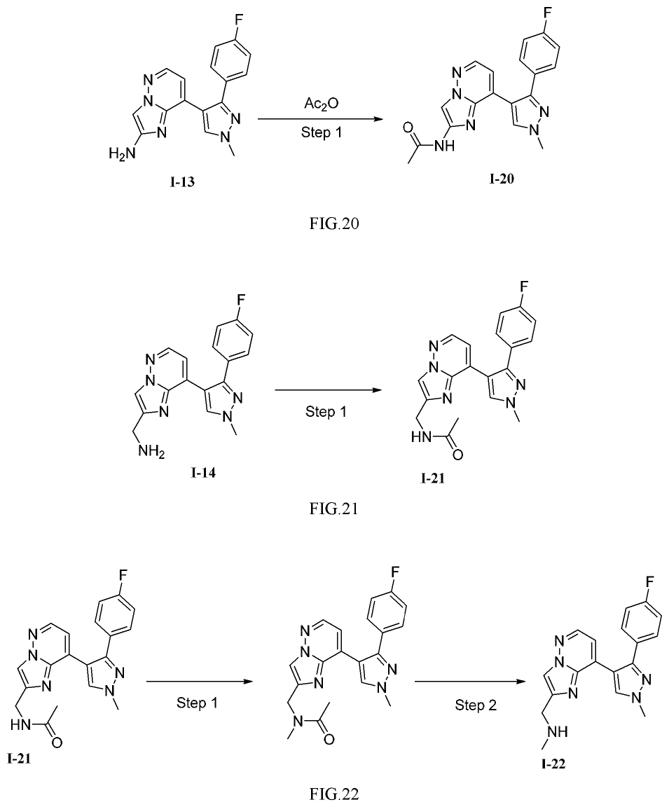

FIG. 20 illustrates the synthetic scheme of compound I-20. As shown in FIG. 20, the specific synthesis steps are as follows:

### Step 1

To a stirred solution of 8-[3-(4-fluorophenyl)-1-methylpyrazol-4-yl]imidazo[1,2-b]pyridazin-2-amine (30 mg, 0.1 mmol, 1.0 equiv) and TEA (30 mg, 0.29 mmol, 3.0 equiv) in DCM (3 mL) were added DMAP (2.4 mg, 0.02 mmol, 0.2 equiv) and Ac₂O (11 mg, 0.11 mmol, 1.1 equiv) at room temperature under nitrogen atmosphere. The resulting mixture was stirred for 3 h at room temperature under nitrogen atmosphere. The resulting mixture was concentrated under reduced pressure. The residue was purified by Prep-HPLC with the following conditions (Column: X Select CSH Prep C18 OBD Column, 19*250 mm, 5 um; Mobile Phase A: water (0.1% FA), Mobile Phase B: ACN; Flow rate: 25 mL/min; Gradient: 18 B to 48 B in 7 min, 254/220 nm; RT₁:6.22 min; Injection Volume: 0.4 ml; Number Of Runs: 8) to afford N-[8-[3-(4-fluorophenyl)-1-methylpyrazol-4-yl]imidazo[1,2-b]pyridazin-2-yl]acetamide (6.7 mg, 20%) as an off-white solid. H NMR (300 MHz, CDCl₃) δ 8.49 (s, 1H), 8.42 (s, 1H), 8.14 (br, 1H), 8.06 (d, J=5.0 Hz, 1H), 7.53-7.46 (m, 2H), 7.14-7.06 (m, 2H), 6.71 (d, J=5.0 Hz, 1H), 4.02 (s, 3H), 2.23 (s, 3H). LC/MS (ESI, m/z): [(M+1)]⁺=351.

### Example 1-21 Preparation of Compound I-21

FIG. 21 illustrates the synthetic scheme of compound I-21. As shown in FIG. 21, the specific synthesis steps are as follows:

### Step 1

To a stirred solution of 1-[8-[3-(4-fluorophenyl)-1-methylpyrazol-4-yl]imidazo[1,2-b]pyridazin-2-yl]methanamine(50.00 mg, 0.155 mmol, 1.00 equiv) and TEA (23.54 mg, 0.233 mmol, 1.5 equiv) in DCM (0.50 mL) was added acetyl chloride(14.61 mg, 0.186 mmol, 1.20 equiv) at 0° C. under air atmosphere. The resulting mixture was stirred for 1 h at room temperature under air atmosphere. The reaction was monitored by LCMS. The residue was purified by reverse flash chromatography with the following conditions: column, C18 silica gel; mobile phase, ACN in 0.1% FA aq., 10% to 50% gradient in 20 min; detector, UV 254 nm to afford N-([8-[3-(4-fluorophenyl)-1-methylpyrazol-4-yl]imidazo[1,2-b]pyridazin-2-yl]methyl)acetamide (20 mg, 35.39%) as an off-white solid. ¹H NMR (300 MHz, Chloroform-d) δ 8.69 (s, 1H), 8.10 (d, J=5.0 Hz, 1H), 7.95 (s, 1H), 7.57-7.47 (m, 2H), 7.13 (t, J=8.6 Hz, 2H), 6.76 (d, J=4.9 Hz, 1H), 6.25 (s, 1H), 4.69 (d, J=4.7 Hz, 2H), 4.08 (s, 3H), 2.09 (s, 3H). LC/MS (ESI, m/z): [(M+1)]⁺=365.1.

### Example 1-22 Preparation of Compound I-22

FIG. 22 illustrates the synthetic scheme of compound I-22. As shown in FIG. 22, the specific synthesis steps are as follows:

### Step 1: N-([8-[3-(4-fluorophenyl)-1-methylpyrazol-4-yl]imidazo[1,2-b]pyridazin-2-yl]methyl)-N-methyl acetamide

To a stirred solution of N-([8-[3-(4-fluorophenyl)-1-methylpyrazol-4-yl]imidazo[1,2-b]pyridazin-2-yl]methyl)acetamide (50.00 mg, 0.137 mmol, 1.00 equiv) in DMF (1.00 mL) was added NaH (10.98 mg, 0.274 mmol, 2 equiv, 60%) at room temperature under nitrogen atmosphere. The resulting mixture was stirred for 30 min at room temperature under nitrogen atmosphere. To the above mixture was added CH₃I (38.95 mg, 0.274 mmol, 2 equiv). The resulting mixture was stirred for additional 16 h at 80° C. under nitrogen atmosphere. The reaction was monitored by LCMS. The residue was purified by reverse flash chromatography with the following conditions: column, C18 silica gel; mobile phase, ACN in 0.1% FA aq., 10% to 50% gradient in 10 min; detector, UV 254 nm to afford N-([8-[3-(4-fluorophenyl)-1-methylpyrazol-4-yl]imidazo[1,2-b]pyridazin-2-yl]methyl)-N-methyl acetamide (50 mg, 96.29%) as a yellow solid. LC/MS (ESI, m/z): [(M+1)]⁺=379.3.

### Step 2

To a stirred solution of N-([8-[3-(4-fluorophenyl)-1-methylpyrazol-4-yl]imidazo[1,2-b]pyridazin-2-yl]methyl)-N-methyl acetamide(50.00 mg, 0.132 mmol, 1.00 equiv) in EtOH(0.50 mL) was added NaOH(4M)(0.50 mL). The resulting mixture was stirred for 4 h at 90° C. under air atmosphere. The reaction was monitored by LCMS. The residue was purified by reverse flash chromatography with the following conditions: column, C18 silica gel; mobile phase, ACN in 0.1% FA aq., 10% to 50% gradient in 10 min; detector, UV 254 nm to afford ([8-[3-(4-fluorophenyl)-1-methylpyrazol-4-yl]imidazo[1,2-b]pyridazin-2-yl]methyl)(methyl)amine (14 mg, 31.50%) as a white solid. H NMR (300 MHz, Chloroform-d) δ 8.91 (s, 1H), 8.53 (s, 1H), 8.05 (d, J=5.3 Hz, 2H), 7.56-7.45 (m, 2H), 7.19-7.07 (m, 2H), 6.73 (d, J=5.0 Hz, 1H), 4.25 (s, 2H), 4.07 (s, 3H), 2.69 (s, 3H). LC/MS (ESI, m/z): [(M+1)]⁺=337.15.

### Example 1-23 Preparation of Compound I-23

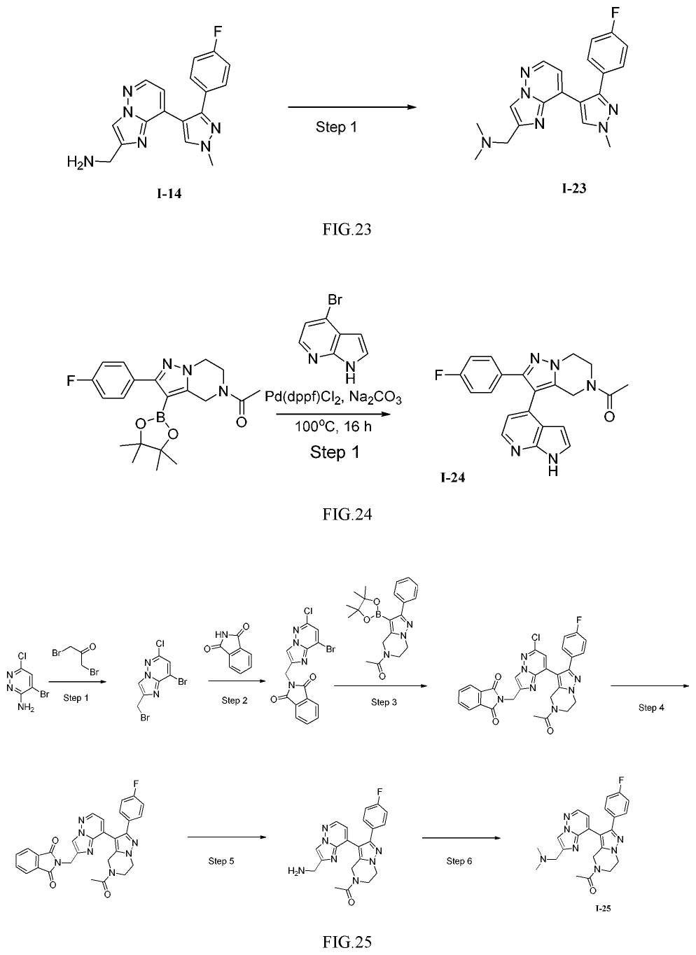

FIG. 23 illustrates the synthetic scheme of compound I-23. As shown in FIG. 23, the specific synthesis steps are as follows:

### Step 1

To a stirred solution of (8-(3-(4-fluorophenyl)-1-methyl-1H-pyrazol-4-yl)imidazo[1,2-b]pyridazin-2-yl)methanamine (44.11 mg, 0.137 mmol, 1.00 equiv) in DMF (1.00 mL) was added NaH (10.98 mg, 0.274 mmol, 2 equiv, 60%) at room temperature under nitrogen atmosphere. The resulting mixture was stirred for 30 min at room temperature under nitrogen atmosphere. To the above mixture was added CH₃I (38.95 mg, 0.274 mmol, 2 equiv). The resulting mixture was stirred for additional 16 h at 80° C. under nitrogen atmosphere. The reaction was monitored by LCMS. The residue was purified by reverse flash chromatography with the following conditions: column, C18 silica gel; mobile phase, ACN in 0.1% FA aq., 10% to 50% gradient in 10 min; detector, UV 254 nm to afford 1-(8-(3-(4-fluorophenyl)-1-methyl-1H-pyrazol-4-yl)imidazo[1,2-b]pyridazin-2-yl)-N,N-dimethyl methanamine (12 mg, 25%) as a yellow solid. LC/MS (ESI, m/z): [(M+1)]⁺=351.4.

### Example 1-24 Preparation of Compound I-24

FIG. 24 illustrates the synthetic scheme of compound I-24. As shown in FIG. 24, the specific synthesis steps are as follows:

### Step 1

To a solution of 1-[2-(4-fluorophenyl)-3-(4,4,5,5-tetramethyl-1,3,2-dioxaborolan-2-yl)-4H,6H,7H-pyrazolo[1,5-a]pyrazin-5-yl]ethanone (1000 mg, 2.5958 mmol) in 1,4-Dioxane/H₂O=10:1 (30 mL), 4-bromo-1H-pyrrolo[2,3-b]pyridine (767.18 mg, 3.8937 mmol), Na₂CO₃ (825.46 mg, 7.7874 mmol), Pd(dppf)Cl₂ (379.51 mg, 0.5191 mmol) was added. The reaction mixture was stirred at 100° C. for 16 h. The reaction was complete detected by lcms. The solution was filtered, filtrate was collected. The reaction mixture was concentrated under pressure. The crude material was added to a silica gel column and was eluted with DCM/MeOH (10:1). Chemical Formula: calculated for (M+H+) C₂₁H₁₈FN₅O: 375.1, Found: 376.0.

### Example 1-25 Preparation of Compound I-25

FIG. 25 illustrates the synthetic scheme of compound I-25. As shown in FIG. 25, the specific synthesis steps are as follows:

### Step 1: 8-bromo-2-(bromomethyl)-6-chloroimidazo[1,2-b]pyridazine

To a stirred mixture of 4-bromo-6-chloropyridazin-3-amine (12.05 g, 57.810 mmol, 1.00 equiv) in DME (100.00 mL) were added 2-propanone, 1,3-dibromo-(24.96 g, 115.624 mmol, 2.00 equiv) at room temperature under air atmosphere. The solution was stirred at 90° C. for 2 h. The residue was purified by silica gel column chromatography, eluted with PE/EA (5:1) to afford 8-bromo-2-(bromomethyl)-6-chloroimidazo[1,2-b]pyridazine(7.4 g, 39.34%) as a white solid. LC/MS (ESI, m/z): [(M+1)]⁺=324.1.

### Step 2: 2-([8-bromo-6-chloroimidazo[1,2-b]pyridazin-2-yl]methyl)isoindole-1,3-dione

To a stirred solution of 8-bromo-2-(bromomethyl)-6-chloroimidazo[1,2-b]pyridazine(13.30 g, 40.874 mmol, 1.00 equiv) and phthalimide(6.62 g, 0.045 mmol, 1.10 equiv) in dioxane(200.00 mL) was added K₂CO₃ (11.30 g, 81.748 mmol, 2.00 equiv) at room temperature under air atmosphere. The mixture was stirred for 1 h at 80° C., the reaction was monitored by LCMS. The reaction mixture was concentrated under reduced pressure. The residue was purified by reverse flash chromatography with the following conditions: column, C18 silica gel; mobile phase, ACN in 0.1% FA, 30% to 50% gradient in 20 min; detector, UV 254 nm. to afford 2-([8-bromo-6-chloroimidazo[1,2-b]pyridazin-2-yl]methyl)isoindole-1,3-dione (10 g, 62.47%) as a white solid. LC/MS (ESI, m/z): [(M+1)]⁺=393.1.

### Step 3: 2-((8-(5-acetyl-2-(4-fluorophenyl)-4,5,6,7-tetrahydropyrazolo[1,5-a]pyrazin-3-yl)-6-chloroimidazo[1,2-b]pyridazin-2-yl)methyl)isoindoline-1,3-dione

To a stirred solution of 2-([8-bromo-6-chloroimidazo[1,2-b]pyridazin-2-yl]methyl)isoindole-1,3-dione(5.70 g, 14.555 mmol, 1.00 equiv) and 1-(2-(4-fluorophenyl)-3-(4,4,5,5-tetramethyl-1,3,2-dioxaborolan-2-yl)-6,7-dihydropyrazolo[1,5-a]pyrazin-5(4H)-yl)ethan-1-one (8.85 g, 0.023 mmol, 1.50 equiv) in Toluene(90.00 mL) were added Pd(dppf)Cl₂·CH₂Cl₂ (1.19 g, 1.456 mmol, 0.10 equiv) and Cs₂CO₃ (14.23 g, 43.665 mmol, 3.00 equiv) at room temperature under nitrogen atmosphere. The resulting mixture was stirred for 2 h at 100° C. under nitrogen atmosphere. The reaction was monitored by LCMS. The residue was purified by reverse flash chromatography with the following conditions: column, C18 silica gel; mobile phase, ACN in 0.1% FA, 10% to 50% gradient in 10 min; detector, UV 254 nm to afford 2-((8-(5-acetyl-2-(4-fluorophenyl)-4,5,6,7-tetrahydropyrazolo[1,5-a]pyrazin-3-yl)-6-chloroimidazo[1,2-b]pyridazin-2-yl)methyl)isoindoline-1,3-dione (2.34 g, 28.22%) as a yellow solid. LC/MS (ESI, m/z): [(M+1)]±=570.9.

### Step 4: 2-((8-(5-acetyl-2-(4-fluorophenyl)-4,5,6,7-tetrahydropyrazolo[1,5-a]pyrazin-3-yl)imidazo[1,2-b]pyridazin-2-yl)methyl)isoindoline-1,3-dione

To a stirred solution of 2-((8-(5-acetyl-2-(4-fluorophenyl)-4,5,6,7-tetrahydropyrazolo[1,5-a]pyrazin-3-yl)-6-chloroimidazo[1,2-b]pyridazin-2-yl)methyl)isoindoline-1,3-dione (2.34 g, 4.108 mmol, 1.00 equiv) in EtOH (60.00 mL) was added Pd/C (437.14 mg, 0.411 mmol, 0.10 equiv, 10%). The resulting mixture was stirred for 48 h at 50° C. under hydrogen atmosphere. The reaction was monitored by LCMS. The resulting mixture was filtered, the filter cake was washed with DCM (3×30 mL). The filtrate was concentrated under reduced pressure. This resulted in 2-((8-(5-acetyl-2-(4-fluorophenyl)-4,5,6,7-tetrahydropyrazolo[1,5-a]pyrazin-3-yl)imidazo[1,2-b]pyridazin-2-yl)methyl)isoindoline-1,3-dione (1 g, 53.81%) as a yellow solid. The crude product was used to the next step directly without further purification. LC/MS (ESI, m/z): [(M+1)]±=536.5.

### Step 5: 1-(3-(2-(aminomethyl)imidazo[1,2-b]pyridazin-8-yl)-2-(4-fluorophenyl)-6,7-dihydropyrazolo[1, 5-a]pyrazin-5(4H)-yl)ethan-1-one

To a stirred solution of 2-((8-(5-acetyl-2-(4-fluorophenyl)-4,5,6,7-tetrahydropyrazolo[1,5-a]pyrazin-3-yl)imidazo[1,2-b]pyridazin-2-yl)methyl)isoindoline-1,3-dione (118.00 mg, 0.221 mmol, 1.00 equiv) in EtOH (1.00 mL) was added N₂H₄·H₂O (66.39 mg, 1.326 mmol, 6 equiv). The resulting mixture was stirred for 1 h at 80° C. under air atmosphere. The reaction was monitored by LCMS. The residue was purified by reverse flash chromatography with the following conditions: column, C18 silica gel; mobile phase, ACN in 0.1% FA aq., 10% to 50% gradient in 25 min; detector, UV 254 nm to afford 1-(3-(2-(aminomethyl)imidazo[1,2-b]pyridazin-8-yl)-2-(4-fluorophenyl)-6,7-dihydropyrazolo[1, 5-a]pyrazin-5(4H)-yl)ethan-1-one (62.6 mg, 70%) as an off-white solid.

### Step 6

To a stirred solution of 1-(3-(2-(aminomethyl)imidazo[1,2-b]pyridazin-8-yl)-2-(4-fluorophenyl)-6,7-dihydropyrazolo[1, 5-a]pyrazin-5(4H)-yl)ethan-1-one (55.00 mg, 0.137 mmol, 1.00 equiv) in DMF (1.00 mL) was added NaH (10.98 mg, 0.274 mmol, 2 equiv, 60%) at room temperature under nitrogen atmosphere. The resulting mixture was stirred for 30 min at room temperature under nitrogen atmosphere. To the above mixture was added CH₃I (38.95 mg, 0.274 mmol, 2 equiv). The resulting mixture was stirred for additional 16 h at 80° C. under nitrogen atmosphere. The reaction was monitored by LCMS. The residue was purified by reverse flash chromatography with the following conditions: column, C18 silica gel; mobile phase, ACN in 0.1% FA aq., 10% to 50% gradient in 10 min; detector, UV 254 nm to afford 1-(3-(2-((dimethylamino)methyl)imidazo[1,2-b]pyridazin-8-yl)-2-(4-fluorophenyl)-6,7-dihydropyrazolo[1,5-a]pyrazin-5(4H)-yl)ethan-1-one (50 mg, 96.29%) as a yellow solid. LC/MS (ESI, m/z): [(M+1)]⁺=434.4.

### Example 1-26 Preparation of Compound I-26

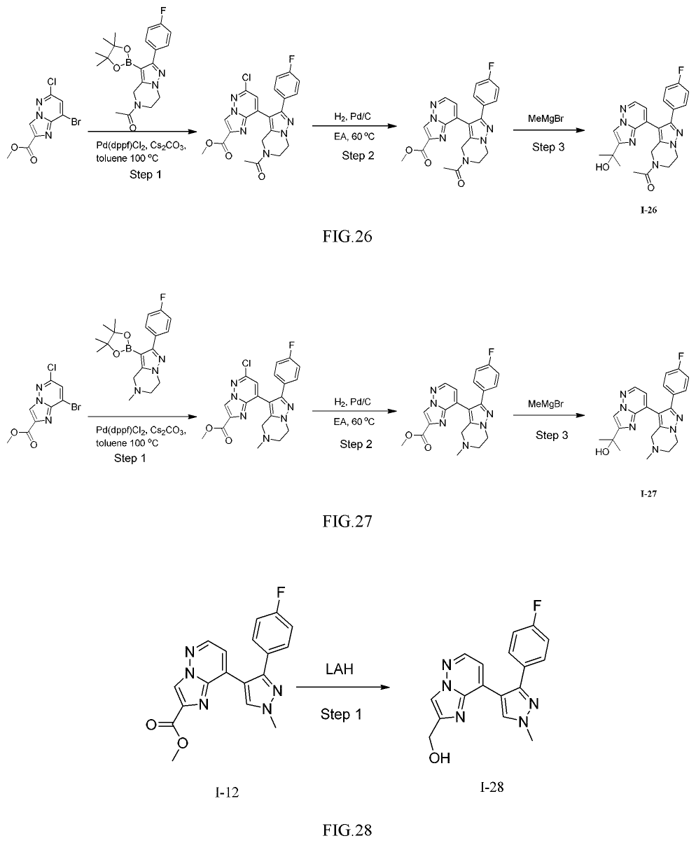

FIG. 26 illustrates the synthetic scheme of compound 1-26. As shown in FIG. 26, the specific synthesis steps are as follows:

### Step 1: methyl 8-(5-acetyl-2-(4-fluorophenyl)-4,5,6,7-tetrahydropyrazolo[1,5-a]pyrazin-3-yl)-6-chloroimidazo[1,2-b]pyridazine-2-carboxylate

To a stirred mixture of methyl 8-bromo-6-chloroimidazo[1,2-b]pyridazine-2-carboxylate (1 g, 3.4 mmol, 1.0 equiv) and 1-(2-(4-fluorophenyl)-3-(4,4,5,5-tetramethyl-1,3,2-dioxaborolan-2-yl)-6,7-dihydropyrazolo[1,5-a]pyrazin-5(4H)-yl)ethan-1-one (1.3 g, 3.4 mmol, 1.0 equiv) in toluene (15 mL) were added Cs₂CO₃ (2.3 g, 6.9 mmol, 2.0 equiv) and Pd(dppf)Cl₂CH₂Cl₂ (280 mg, 0.34 mmol, 0.1 equiv) at room temperature under nitrogen atmosphere. The resulting mixture was stirred for 2 h at 100° C. under nitrogen atmosphere. The resulting mixture was concentrated under reduced pressure. The residue was purified by silica gel column chromatography, eluted with PE/EA (1:2) to afford methyl 8-(5-acetyl-2-(4-fluorophenyl)-4,5,6,7-tetrahydropyrazolo[1,5-a]pyrazin-3-yl)-6-chloroimidazo[1,2-b]pyridazine-2-carboxylate (490 mg, 29%) as a light brown solid.

### Step 2: methyl 8-(5-acetyl-2-(4-fluorophenyl)-4,5,6,7-tetrahydropyrazolo[1,5-a]pyrazin-3-yl)imidazo[1,2-b]pyridazine-2-carboxylate

To a solution of methyl methyl 8-(5-acetyl-2-(4-fluorophenyl)-4,5,6,7-tetrahydropyrazolo[1,5-a]pyrazin-3-yl)-6-chloroimidazo[1,2-b]pyridazine-2-carboxylate (468 mg, 1.0 mmol, 1.0 equiv) in EA (10 mL) was added Pd/C (97 mg, 10%) under nitrogen atmosphere. The mixture was hydrogenated at 50° C. for 16 h under hydrogen atmosphere using a hydrogen tyre. The resulting mixture was filtered and the filter cake was washed with MeOH (3×10 mL). The filtrate was concentrated under reduced pressure. The residue was purified by Prep-HPLC with the following conditions (Column: X Select CSH Prep C18 OBD Column, 19*250 mm, 5 um; Mobile Phase A: water (0.05% FA), Mobile Phase B: ACN; Flow rate: 25 mL/min; Gradient: 30 B to 55 B in 7 min, 254/220 nm; RT₁: 6.47 min; Injection Volume: 0.4 ml; Number Of Runs: 8) to afford methyl 8-(5-acetyl-2-(4-fluorophenyl)-4,5,6,7-tetrahydropyrazolo[1,5-a]pyrazin-3-yl)imidazo[1,2-b]pyridazine-2-carboxylate (295 mg, 68%) as an off-white solid.

### Step 3

To a stirred mixture of methyl 8-(5-acetyl-2-(4-fluorophenyl)-4,5,6,7-tetrahydropyrazolo[1,5-a]pyrazin-3-yl)imidazo[1,2-b]pyridazine-2-carboxylate (123 mg, 0.285 mmol, 1.00 equiv) in THF (10.00 mL) was added MeMgBr (0.60 mL, 1.800 mmol, 6.32 equiv) dropwise at 0° C. under air atmosphere. The reaction was reacted for 1 hour at room temperature. The reaction was monitored by LCMS, then quenched by NHCl₄ aq., concentrated under reduced pressure. The residue was purified by reverse flash chromatography with the following conditions: column, C18 silica gel; mobile phase, ACN in 0.1% FA aq., 35% to 55% gradient in 15 min; detector, UV 254 nm. This resulted in 1-(2-(4-fluorophenyl)-3-(2-(2-hydroxypropan-2-yl)imidazo[1,2-b]pyridazin-8-yl)-6,7-dihydropyrazolo[1,5-a]pyrazin-5(4H)-yl)ethan-1-one (29.5 mg, 24.0%) as a light yellow solid.

### Example 1-27 Preparation of Compound I-27

FIG. 27 illustrates the synthetic scheme of compound 1-27. As shown in FIG. 27, the specific synthesis steps are as follows:

### Step 1: methyl 6-chloro-8-(2-(4-fluorophenyl)-5-methyl-4,5,6,7-tetrahydropyrazolo[1,5-a]pyrazin-3-yl)imidazo[1,2-b]pyridazine-2-carboxylate

To a stirred mixture of methyl 8-bromo-6-chloroimidazo[1,2-b]pyridazine-2-carboxylate (1 g, 3.4 mmol, 1.0 equiv) and 1-(2-(4-fluorophenyl)-3-(4,4,5,5-tetramethyl-1,3,2-dioxaborolan-2-yl)-6,7-dihydropyrazolo[1,5-a]pyrazin-5(4H)-yl)ethan-1-one (1.3 g, 3.4 mmol, 1.0 equiv) in toluene (15 mL) were added Cs₂CO₃ (2.3 g, 6.9 mmol, 2.0 equiv) and Pd(dppf)Cl₂CH₂Cl₂ (280 mg, 0.34 mmol, 0.1 equiv) at room temperature under nitrogen atmosphere. The resulting mixture was stirred for 2 h at 100° C. under nitrogen atmosphere. The resulting mixture was concentrated under reduced pressure. The residue was purified by silica gel column chromatography, eluted with PE/EA (1:2) to afford methyl 6-chloro-8-(2-(4-fluorophenyl)-5-methyl-4,5,6,7-tetrahydropyrazolo[1,5-a]pyrazin-3-yl)imidazo[1,2-b]pyridazine-2-carboxylate (460 mg, 28%) as a light brown solid.

### Step 2: methyl 8-(2-(4-fluorophenyl)-5-methyl-4,5,6,7-tetrahydropyrazolo[1,5-a]pyrazin-3-yl)imidazo[1,2-b]pyridazine-2-carboxylate

To a solution of methyl 6-chloro-8-(2-(4-fluorophenyl)-5-methyl-4,5,6,7-tetrahydropyrazolo[1,5-a]pyrazin-3-yl)imidazo[1,2-b]pyridazine-2-carboxylate (440 mg, 1.0 mmol, 1.0 equiv) in EA (10 mL) was added Pd/C (97 mg, 10%) under nitrogen atmosphere. The mixture was hydrogenated at 50° C. for 16 h under hydrogen atmosphere using a hydrogen tyre. The resulting mixture was filtered and the filter cake was washed with MeOH (3×10 mL). The filtrate was concentrated under reduced pressure. The residue was purified by Prep-HPLC with the following conditions (Column: X Select CSH Prep C18 OBD Column, 19*250 mm, 5 um; Mobile Phase A: water (0.05% FA), Mobile Phase B: ACN; Flow rate: 25 mL/min; Gradient: 30 B to 55 B in 7 min, 254/220 nm; RT₁: 6.47 min; Injection Volume: 0.4 ml; Number Of Runs: 8) to afford methyl 8-(2-(4-fluorophenyl)-5-methyl-4,5,6,7-tetrahydropyrazolo[1,5-a]pyrazin-3-yl)imidazo[1,2-b]pyridazine-2-carboxylate (280 mg, 69%) as an off-white solid.

### Step 3

To a stirred mixture of methyl 8-(2-(4-fluorophenyl)-5-methyl-4,5,6,7-tetrahydropyrazolo[1,5-a]pyrazin-3-yl)imidazo[1,2-b]pyridazine-2-carboxylate (123 mg, 0.285 mmol, 1.00 equiv) in THF (10.00 mL) was added MeMgBr (0.60 mL, 1.800 mmol, 6.32 equiv) dropwise at 0° C. under air atmosphere. The reaction was reacted for 1 hour at room temperature. The reaction was monitored by LCMS, then quenched by NHCl₄ aq., concentrated under reduced pressure. The residue was purified by reverse flash chromatography with the following conditions: column, C18 silica gel; mobile phase, ACN in 0.1% FA aq., 35% to 55% gradient in 15 min; detector, UV 254 nm. This resulted in 2-(8-(2-(4-fluorophenyl)-5-methyl-4,5,6,7-tetrahydropyrazolo[1,5-a]pyrazin-3-yl)imidazo[1,2-b]pyridazin-2-yl)propan-2-ol (29.5 mg, 24.0%) as a light yellow solid.

### Example 1-28 Preparation of Compound I-28

FIG. 28 illustrates the synthetic scheme of compound 1-28. As shown in FIG. 28, the specific synthesis steps are as follows:

### Step 1

To a solution of methyl 8-[3-(4-fluorophenyl)-1-methylpyrazol-4-yl]imidazo[1,2-b]pyridazine-2-carboxylate [100 mg, 0.2846 mmol] in THF [5 mL], then was added LAH [32.44 mg, 0.8538 mmol], The reaction mixture was stirred at 25° C. for 2 h. The reaction was monitored by LCMS, then quenched by H₂O, concentrated under reduced pressure. The residue was purified by reverse flash chromatography with the following conditions: column, C18 silica gel; mobile phase, ACN in 0.1% FA aq., 35% to 55% gradient in 15 min; detector, UV 254 nm. This resulted in (8-(3-(4-fluorophenyl)-1-methyl-1H-pyrazol-4-yl)imidazo[1,2-b]pyridazin-2-yl)methanol (29.5 mg, 29.50%) as a light yellow solid.

### Example 1-29 Preparation of Compound I-29

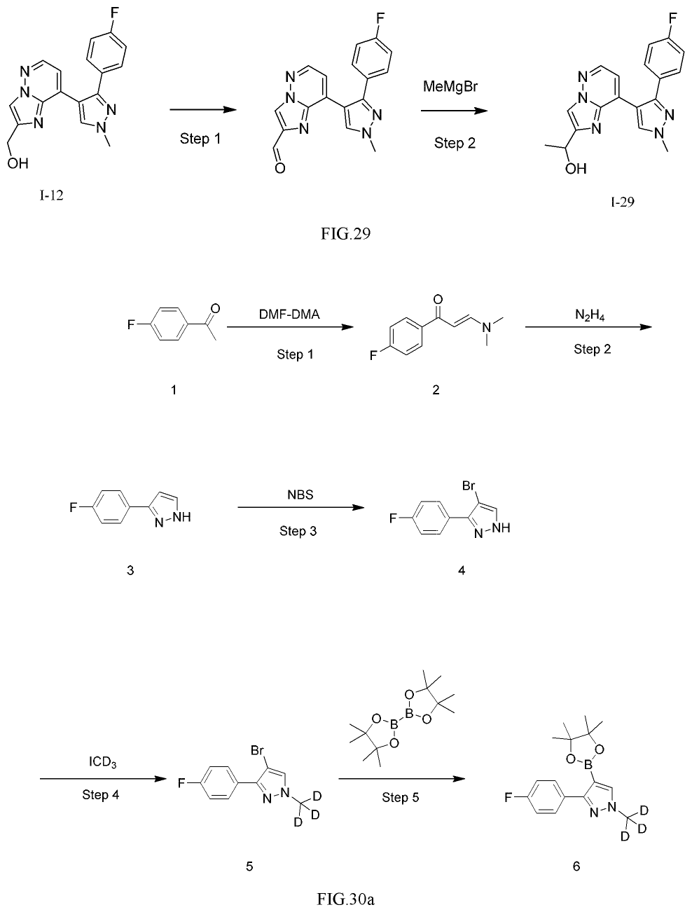

FIG. 29 illustrates the synthetic scheme of compound 1-29. As shown in FIG. 29, the specific synthesis steps are as follows:

### Step 1: 8-(3-(4-fluorophenyl)-1-methyl-1H-pyrazol-4-yl)imidazo[1,2-b]pyridazine-2-carbaldehyde

(8-(3-(4-fluorophenyl)-1-methyl-1H-pyrazol-4-yl)imidazo[1,2-b]pyridazin-2-yl)methanol (985 mg, 3.05 mmol), 4-methylmorpholine N-oxide (465 mg, 3.97 mmol), and 4 Å sieves (600 mg) were dissolved in anhydrous methylene chloride (30 mL) and placed under argon atmosphere. Tetrapropylammonium perruthenate (107 mg, 0.305 mmol) was added portionwise as a solid to the stirring solution, the resulting solution was then stirred at 25° C. for 16 hours. The crude reaction mixture was filtered over a celite pad and concentrated in vacuo. The crude product was purified using silica gel chromatography (300 g, using 15-75% ethyl acetate in hexane gradient) to afford the 8-(3-(4-fluorophenyl)-1-methyl-1H-pyrazol-4-yl)imidazo[1,2-b]pyridazine-2-carbaldehyde as a colorless oil.

### Step 2

To a stirred mixture of 8-(3-(4-fluorophenyl)-1-methyl-1H-pyrazol-4-yl)imidazo[1,2-b]pyridazine-2-carbaldehyde (100 mg, 0.311 mmol, 1.00 equiv) in THF (10.00 mL) was added MeMgBr (0.60 mL, 1.800 mmol, 6.32 equiv) dropwise at 0° C. under air atmosphere. The reaction was reacted for 1 hour at room temperature. The reaction was monitored by LCMS, then quenched by NHCl₄ aq., concentrated under reduced pressure. The residue was purified by reverse flash chromatography with the following conditions: column, C18 silica gel; mobile phase, ACN in 0.1% FA aq., 35% to 55% gradient in 15 min; detector, UV 254 nm. This resulted in 1-(8-(3-(4-fluorophenyl)-1-methyl-1H-pyrazol-4-yl)imidazo[1,2-b]pyridazin-2-yl)ethan-1-ol (29.5 mg, 28%) as a light yellow solid.

### Example 1-30 Preparation of Compound I-30

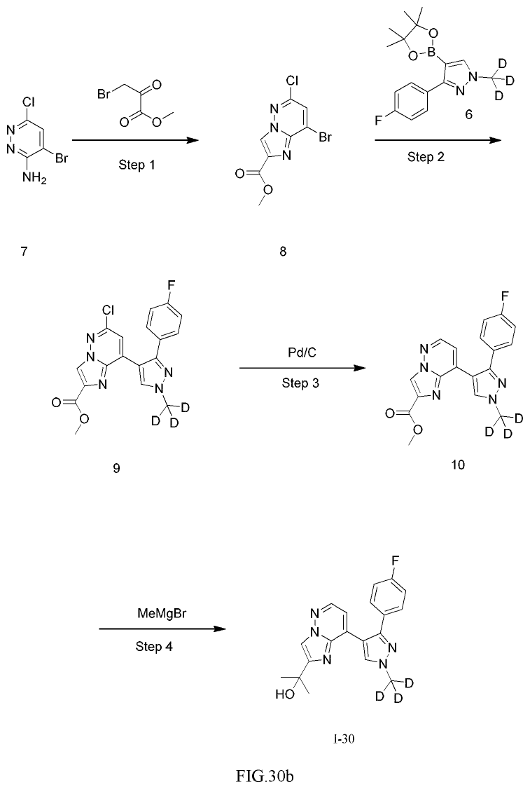

FIG. 30a and FIG. 30b illustrate the synthetic scheme of compound 1-30. As shown in FIG. 30a, the specific synthesis steps are as follows:

### Step 1: (E)-3-(dimethylamino)-1-(4-fluorophenyl)prop-2-en-1-one (2)

A solution of 1-(4-fluorophenyl)ethan-1-one (5.00 g, 36.20 mmol, 1.0 eq) in DMF-DMA (20 ml) was stirred at 100° cfor 2 hrs. After reaction completed, The mixture was concentrated to give the crude product (E)-3-(dimethylamino)-1-(4-fluorophenyl)prop-2-en-1-one (6.92 g, 35.81 mmol, 98.92%) as a yellow solid, which used for next step directly. Chemical Formula: calculated for (M+H⁺) C₁₁H₁₂FNO: 193.22, Found: 194.

### Step 2: 3-(4-fluorophenyl)-1H-pyrazole (3)

To a solution of (E)-3-(dimethylamino)-1-(4-fluorophenyl)prop-2-en-1-one (6.80 g, 35.19 mmol, 1.0 eq) in EtOH (30 ml) was added Hydrazine monohydrate, 98+% (2.29 g, 45.75 mmol, 1.3 eq) at rt, after addition, the mixture was stirred at 85° C. for 12 hrs. LCMS showed SM disappeared, the mixture was concentrated and purified by silical gel column to give 3-(4-fluorophenyl)-1H-pyrazole (4.57 g, 28.18 mmol, 80.08%) as a yellow solid. Chemical Formula: calculated for (M+H⁺) C₉H₇FN₂: 162.17, Found: 163.

### Step 3: 4-bromo-3-(4-fluorophenyl)-1H-pyrazole (4)

To a solution of 3-(4-fluorophenyl)-1H-pyrazole (4.50 g, 27.75 mmol, 1.0 eq) in DMF (25 mL) was added NBS (5.92 g, 33.30 mmol, 1.2 eq). After addition, the mixture was stirred at 60° C. for 12 hrs. LCMS showed SM disappeared. The mixture was poured into water(200.0 mL), extracted with EtOAc(10 0 mL×3 times), the combined organic phases were washed with water(50.0 mL) and brine(50.0 mL), dried by Na₂SO₄, concentrated and purified by silical gel column to give the crude 4-bromo-3-(4-fluorophenyl)-1H-pyrazole (5.04 g, 20.91 mmol, 75.36%) as a yellow solid. Chemical Formula: calculated for (M+H⁺) C₉H₆BrFN₂: 241.06, Found: 241,243.

### Step 4:4-bromo-3-(4-fluorophenyl)-1-(methyl-d3)-1H-pyrazole (5)

To a solution 4-bromo-3-(4-fluorophenyl)-1H-pyrazole of (4.90 g, 20.33 mmol, 1.0 eq) in DMF (50 mL) was added iodomethane-d3 (3.24 g, 22.36 mmol, 1.1 eq) and CS₂CO3 (13.25 g, 40.66 mmol, 2.0 eq) at rt. after addition, the mixture was stirred at 10c for 10 hrs. LCMS showed SM disappeared. The mixture was poured into water(250.0 mL), extracted with EtOAc(100.0 mL×3 times), the combined organic phase was washed with water(50 mL) and brine(50 mL), dried by Na₂SO₄, concentrated to give 4-bromo-3-(4-fluorophenyl)-1-(methyl-d3)-1H-pyrazole (3.95 g, 15.31 mmol, 75.33%) Chemical Formula: calculated for (M+H)C₁₀H₅D₃BrFN₂:258.11, Found: 258,260.

### Step 5: 3-(4-fluorophenyl)-1-(methyl-d3)-4-(4,4,5,5-tetramethyl-1,3,2-dioxaborolan-2-yl)-1H-pyrazole (6)

To a solution of 4-bromo-3-(4-fluorophenyl)-1-(methyl-d3)-1H-pyrazole(3.80 g, 14.72 mmol, 1.0 eq) in dioxane (38 mL) and was added 4,4,4′,4′,5,5,5′,5′-octamethyl-2,2′-bi(1,3,2-dioxaborolane) (4.49 g, 17.66 mmol, 1.2 eq), Pd(dppf)Cl2 (538.54 mg, 0.736 mmol) and AcOK (2.89 g, 29.44 mmol, 2.0 eq) at rt, after addition, the mixture was stirred at 100° C. for 6 hrs under N₂, the mixture was concentrated and purified by Flash (Pet. ether:EtOAc=60:40) to give 3-(4-fluorophenyl)-1-(methyl-d3)-4-(4,4,5,5-tetramethyl-1,3,2-dioxaborolan-2-yl)-1H-pyrazole (3.75 g, 12.29 mmol, 83.49%) as a white solid. Chemical Formula: calculated for (M+H⁺)C₁₆H₁₇D₃BFN₂O₂: 305.17, Found: 306.

As shown in FIG. 30b, the specific synthesis steps are as follows:

### Step 1: methyl 8-bromo-6-chloroimidazo[1,2-b]pyridazine-2-carboxylate (8)

To a stirred mixture of 4-bromo-6-chloropyridazin-3-amine (5.00 g, 23.99 mmol, 1.0 equiv) and methyl 3-bromo-2-oxopropanoate (17.37 g, 95.96 mmol, 4.0 equiv) in DME (50 mL) at room temperature under air atmosphere. The reaction stirred for 16 hrs at 90° C. The resulting mixture was concentrated under reduced pressure. The residue was purified by silica gel column chromatography, eluted with PE/EA (3:1) to afford methyl 8-bromo-6-chloroimidazo[1,2-b]pyridazine-2-carboxylate (5.73 g, 19.72 mmol, 82.20%) as an off-white solid. Chemical Formula: calculated for (M+H⁺)C₈H₅BrClN₃O₂: 290.50, Found: 290, 292.

### Step 2: methyl 6-chloro-8-(3-(4-fluorophenyl)-1-(methyl-d3)-1H-pyrazol-4-yl)imidazo[1,2-b]pyridazine-2-carboxylate (9)

To a stirred mixture of methyl 8-bromo-6-chloroimidazo[1,2-b]pyridazine-2-carboxylate (3.50 g, 12.07 mmol, 1.0 equiv) and 3-(4-fluorophenyl)-1-(methyl-d3)-4-(4,4,5,5-tetramethyl-1,3,2-dioxaborolan-2-yl)-1H-pyrazole (3.68 g, 12.07 mmol, 1.0 equiv) in DME (60 mL) and water (10 ml) were added Cs₂CO₃ (7.87 g, 24.14 mmol, 2.0 equiv) and Pd(dppf)Cl₂CH₂Cl₂ (995 mg, 1.207 mmol, 0.1 equiv) at room temperature under nitrogen atmosphere. The resulting mixture was stirred for 2 h at 100° C. under nitrogen atmosphere. The resulting mixture was concentrated under reduced pressure. The residue was purified by silica gel column chromatography, eluted with PE/EA (1:2) to afford methyl 6-chloro-8-(3-(4-fluorophenyl)-1-(methyl-d3)-1H-pyrazol-4-yl)imidazo[1,2-b]pyridazine-2-carboxylate (3.53 g, 9.08 mmol, 75.26%) as a light brown solid. Chemical Formula: calculated for (M+H⁺)C₁₈H₁₀D₃ClFN₅O₂:388.80, Found: 389.

### Step 3: methyl 8-(3-(4-fluorophenyl)-1-(methyl-d3)-1H-pyrazol-4-yl)imidazo[1,2-b]pyridazine-2-carboxylate(10)

To a solution of methyl 6-chloro-8-(3-(4-fluorophenyl)-1-(methyl-d3)-1H-pyrazol-4-yl)imidazo[1,2-b]pyridazine-2-carboxylate (3.40 g, 8.74 mmol, 1.0 equiv) in EA (35 mL) was added 10% Pd/C (340 mg, 10% w/w) under nitrogen atmosphere. The mixture was hydrogenated at 25° C. for 16 hrs under hydrogen atmosphere. After reaction completed, the resulting mixture was filtered and the filter cake was washed with MeOH (50 mL×3 times). The filtrate was concentrated under reduced pressure. The residue was purified by Prep-HPLC to afford methyl 8-(3-(4-fluorophenyl)-1-(methyl-d3)-11H-pyrazol-4-yl)imidazo[1,2-b]pyridazine-2-carboxylate(2.75 g, 7.76 mmol, 88.78%) as an off-white solid. Chemical Formula: calculated for (M+H⁺) C₁₈H₁₁D₃FN₅O₂:354.36, Found: 355.

### Step 4:2-(8-(3-(4-fluorophenyl)-1-(methyl-d3)-1H-pyrazol-4-yl)imidazo[1,2-b]pyridazin-2-yl)propan-2-ol (I-30

To a stirred mixture of methyl 8-(3-(4-fluorophenyl)-1-(methyl-d3)-11H-pyrazol-4-yl)imidazo[1,2-b]pyridazine-2-carboxylate (500 mg, 1.41 mmol, 1.00 equiv) in THF (10.00 mL) was added MeMgBr (3.53 mL, 7.05 mmol, 5.0 equiv) dropwise at 0° C. under N₂ atmosphere. The reaction was reacted for 1 hour at room temperature. The reaction was monitored by LCMS, then quenched by NHCl₄ aq, concentrated under reduced pressure. The residue was purified by reverse flash chromatography to afford 2-(8-(3-(4-fluorophenyl)-1-(methyl-d3)-1H-pyrazol-4-yl)imidazo[1,2-b]pyridazin-2-yl)propan-2-ol (344.22 mg, 0.96 mmol, 68.32%) as a light yellow solid. Chemical Formula: calculated for (M+H⁺)Cl₉H₁₅D₃FN₅O: 354.40, Found: 355.

### Example 1-31 Preparation of Compound I-31

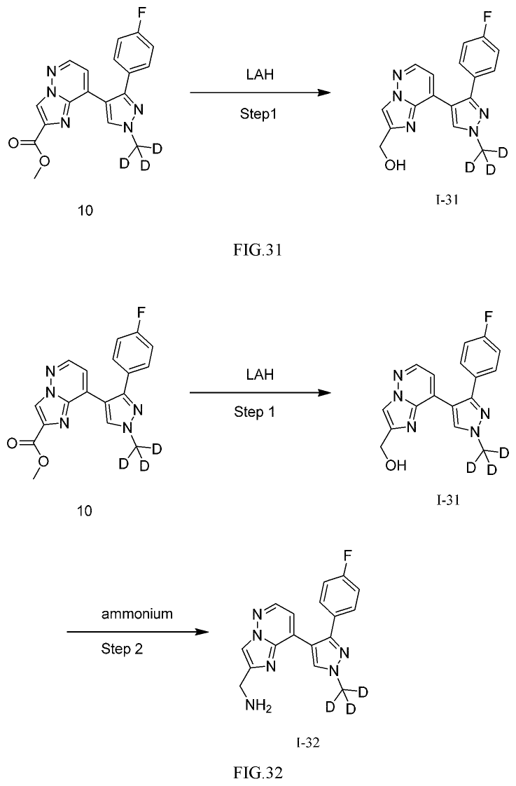

FIG. 31 illustrates the synthetic scheme of compound I-31. As shown in FIG. 31, the specific synthesis steps are as follows:

### Step 1:(8-(3-(4-fluorophenyl)-1-(methyl-d3)-1H-pyrazol-4-yl)imidazo[1,2-b]pyridazin-2-yl)methanol (I-31)

To a stirred mixture of methyl 8-(3-(4-fluorophenyl)-1-(methyl-d3)-11H-pyrazol-4-yl)imidazo[1,2-b]pyridazine-2-carboxylate (1.80 g, 5.08 mmol, 1.00 equiv) in THF (15.00 mL) was added H₄AlLi (385.57 mg, 10.16 mmol, 2.0 equiv) at 0° C. under N₂ atmosphere. The reaction was reacted for 1 hour at room temperature. The reaction was monitored by LCMS, then quenched by NHCl₄ aq, extracted with EtOAc(20.0 ml×3 times), the combined organic phase was washed with water(10 mL) and brine(10 mL), dried by Na₂SO₄, concentrated under reduced pressure. The residue was purified by reverse flash chromatography to afford (8-(3-(4-fluorophenyl)-1-(methyl-d3)-1H-pyrazol-4-yl)imidazo[1,2-b]pyridazin-2-yl)methanol (1.25 g, 3.83 mmol, 75.31%) as a oil. Chemical Formula: calculated for (M+H⁺)C₁₇H₁₁D₃FN₅O: 326.35, Found: 327.

### Example 1-32 Preparation of Compound I-32

FIG. 32 illustrates the synthetic scheme of compound I-32. As shown in FIG. 32, the specific synthesis steps are as follows:

### Step 1:(8-(3-(4-fluorophenyl)-1-(methyl-d3)-1H-pyrazol-4-yl)imidazo[1,2-b]pyridazin-2-yl)methanol (I-31)

To a stirred mixture of methyl 8-(3-(4-fluorophenyl)-1-(methyl-d3)-11H-pyrazol-4-yl)imidazo[1,2-b]pyridazine-2-carboxylate (1.80 g, 5.08 mmol, 1.00 equiv) in THF (15.00 mL) was added H₄AlLi (385.57 mg, 10.16 mmol, 2.0 equiv) at 0° C. under N₂ atmosphere. The reaction was reacted for 1 hour at room temperature. The reaction was monitored by LCMS, then quenched by NHCl₄ aq, extracted with EtOAc(20.0 ml×3 times), the combined organic phase was washed with water(10 mL) and brine(10 mL), dried by Na₂SO₄, concentrated under reduced pressure. The residue was purified by reverse flash chromatography to afford (8-(3-(4-fluorophenyl)-1-(methyl-d3)-1H-pyrazol-4-yl)imidazo[1,2-b]pyridazin-2-yl)methanol (1.25 g, 3.83 mmol, 75.31%) as a oil. Chemical Formula: calculated for (M+H⁺)Cl₇H₁₁D₃FN₅O: 326.35, Found: 327.

### Step 2:(8-(3-(4-fluorophenyl)-1-(methyl-d3)-1H-pyrazol-4-yl)imidazo[1,2-b]pyridazin-2-yl)methanamine (I-32)

(8-(3-(4-fluorophenyl)-1-(methyl-d3)-1H-pyrazol-4-yl)imidazo[1,2-b]pyridazin-2-yl)methanol (450 mg, 1.38 mmol, 1.00 equiv) was added in ammonium solution in MeOH (10 ml). The reaction was reacted for 10 hrs at 40° C. The reaction was monitored by LCMS, The reaction system was concentrated under reduced pressure. The residue was purified by reverse flash chromatography to afford (8-(3-(4-fluorophenyl)-1-(methyl-d3)-1H-pyrazol-4-yl)imidazo[1,2-b]pyridazin-2-yl)methanami ne (292.83 mg, 0.90 mmol, 65.26%) as a oil. Chemical Formula: calculated for (M+H⁺)C₁₇H₁₂D₃FN₆:325.37, Found: 326.

### Example 1-33 Preparation of Compound I-33

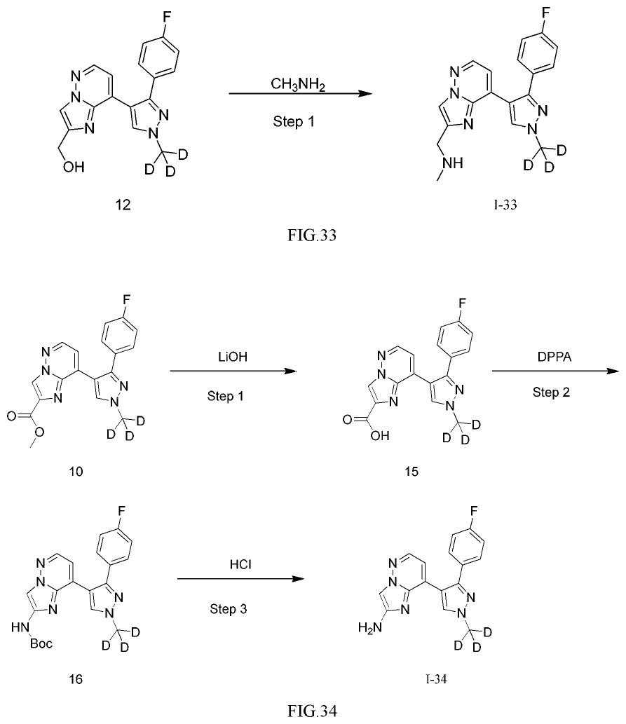

FIG. 33 illustrates the synthetic scheme of compound I-33. As shown in FIG. 33, the specific synthesis steps are as follows:

### Step 1:1-(8-(3-(4-fluorophenyl)-1-(methyl-d3)-1H-pyrazol-4-yl)imidazo[1,2-b]pyridazin-2-yl)-N-methylmethanamine (I-33)

(8-(3-(4-fluorophenyl)-1-(methyl-d3)-1H-pyrazol-4-yl)imidazo[1,2-b]pyridazin-2-yl)methanol (450 mg, 1.38 mmol, 1.00 equiv) was added in methylamine solution in MeOH (10 ml). The reaction was reacted for 10 hrs at 40° C., The reaction was monitored by LCMS, The reaction system was concentrated under reduced pressure. The residue was purified by reverse flash chromatography to afford 1-(8-(3-(4-fluorophenyl)-1-(methyl-d3)-1H-pyrazol-4-yl)imidazo[1,2-b]pyridazin-2-yl)-N-methylmethanamine (329.21 mg, 0.97 mmol, 70.15%) as a white solid. Chemical Formula: calculated for (M+H⁺) C₁₈H₁₄D₃FN₆:339.39, Found: 340.

### Example 1-34 Preparation of Compound I-34

FIG. 34 illustrates the synthetic scheme of compound I-34. As shown in FIG. 34, the specific synthesis steps are as follows:

### Step 1: 8-(3-(4-fluorophenyl)-1-(methyl-d3)-11H-pyrazol-4-yl)imidazo[1,2-b]pyridazine-2-carboxylic acid(15)

To a stirred mixture of methyl 8-(3-(4-fluorophenyl)-1-(methyl-d3)-11H-pyrazol-4-yl)imidazo[1,2-b]pyridazine-2-carboxylate (450 mg, 1.27 mmol, 1.0 equiv) was added in MeOH (10 ml) and water (5 ml), LiOH·H₂O (133.43 mg, 3.18 mmol, 2.5 equiv) was added in. The reaction was reacted for 4 hrs at 35° C. The reaction was monitored by LCMS, The PH of reaction system was adjusted to 5˜6 and extracted with EtOAc(15.0 ml×3 times), the combined organic phase was washed with water(10 mL) and brine(10 mL), dried by Na₂SO₄, The combined organic phase was concentrated under reduced pressure. The residue was purified by reverse flash chromatography to afford 8-(3-(4-fluorophenyl)-1-(methyl-d3)-11H-pyrazol-4-yl)imidazo[1,2-b]pyridazine-2-carboxylic acid (377.77 mg, 1.11 mmol, 87.20%) as a oil. Chemical Formula: calculated for (M+H⁺) C₁₇H₉D₃FN₅O₂:340.33, Found: 341.

### Step 2: tert-butyl (8-(3-(4-fluorophenyl)-1-(methyl-d3)-1H-pyrazol-4-yl)imidazo[1,2-b]pyridazin-2-yl)carbamate (16)

To a stirred mixture of 8-(3-(4-fluorophenyl)-1-(methyl-d3)-1H-pyrazol-4-yl)imidazo[1,2-b]pyridazine-2-carboxylic acid (350 mg, 1.03 mmol, 1.0 equiv) was added in t-BuOH (10 ml), DPPA (341.25 mg, 1.24 mmol, 1.2 equiv) and TEA (261.07 mg, 2.58 mmol, 2.5 equiv) was added in. The reaction was reacted for 4 hrs at 85° C., The reaction was monitored by LCMS, The reaction system was quenched by water and extracted with EtOAc(15.0 ml×3 times), the combined organic phase was washed with water(10 mL) and brine(10 mL), dried by Na₂SO₄, The combined organic phase was concentrated under reduced pressure. The residue was purified by reverse flash chromatography to afford tert-butyl (8-(3-(4-fluorophenyl)-1-(methyl-d3)-1H-pyrazol-4-yl)imidazo[1,2-b]pyridazin-2-yl)carbamate (308.60 mg, 0.75 mmol, 73.18%) as a white solid. Chemical Formula: calculated for (M+H⁺)C₂₁H₁₈D₃FN₆O₂:411.46, Found: 412.

### Step 3:8-(3-(4-fluorophenyl)-1-(methyl-d3)-1H-pyrazol-4-yl)imidazo[1,2-b]pyridazin-2-amine (I-34)

To a stirred mixture of tert-butyl (8-(3-(4-fluorophenyl)-1-(methyl-d3)-1H-pyrazol-4-yl)imidazo[1,2-b]pyridazin-2-yl)carbamate(300 mg, 0.73 mmol, 1.0 equiv) was added in DCM (10 ml), TFA (0.5 ml) was added in. The reaction was reacted for 4 hrs at 25° C. The reaction was monitored by LCMS, The reaction system was concentrated under reduced pressure. The residue was purified by reverse flash chromatography to afford 8-(3-(4-fluorophenyl)-1-(methyl-d3)-1H-pyrazol-4-yl)imidazo[1,2-b]pyridazin-2-amine (155.67 mg, 0.50 mmol, 68.37%) as a white solid. Chemical Formula: calculated for (M+H⁺)C₁₆H₁₀D₃FN₆:311.34, Found: 312.

### Example 2 Assay of Biological Activities

The CK16 kinase assay was performed with a buffer (40 μL, pH 7.5) containing 50 mM Tris (tris-(hydroxymethyl)-aminomethane), 10 mM MgCl₂, 1 mM dithiothreitol, 100 μg/mL BSA (bovine serum albumin) with 10 μM ATP (adenosine triphosphate), 2 nM wild type CK16, and 42 μM peptide substrate PLSRTLpSVASLPGL (Flotow et al., 1990) in the presence of 1 μL of a CK16 inhibitor (e.g., a compound of the present application) or 4% DMSO (e.g., as control). The reaction mixture was incubated for 85 min at 25° C.; detection was carried out as described for the Kinase-Glo Assay (Promega). Luminescent output was measured on the Perkin Elmer Envision plate reader (PerkinElmer, Waltham, MA).

Bmal1-dLuc or Per2-dLuc U2OS cells were suspended in the culture medium (DMEM supplemented with 10% fetal bovine serum, 0.29 mg/mL L-glutamine, 100 units/mL penicillin, and 100 mg/mL streptomycin) and plated onto 96-well white solid-bottom plates at 200 μL (10,000 cells) per well. After 2 days, 100 μL of the explant medium (DMEM supplemented with 2% B27, 10 mM HEPES, 0.38 mg/mL sodium bicarbonate, 0.29 mg/mL L-glutamine, 100 units/mL penicillin, 100 mg/mL streptomycin, 0.1 mg/mL gentamicin, and 1 mM luciferin, pH 7.2) was dispensed to each well, followed by the application of 1 L of a compound of the present application (dissolved in DMSO; final concentration was 0.7% in DMSO). The plate was covered with an optically clear film and set to microplate reader (Infinite M200, Tecan). The luminescence was recorded every 1 h for 3-4 days. The period parameter was obtained from the luminescence rhythm by curve fitting program CellulaRhythm or MultiCycle (Actimetrics), both of which generated similar results.

The CK1δ inhibition results (EC50) are summarized in Table 2.

TABLE 2

| Compound | CK1δ (EC50, μM) |
|---|---|
| I-1 | 15.0 |
| I-4 | 2.7 |
| I-8 | 3.3 |
| I-9 | 2.5 |
| I-10 | 7.9 |
| I-12 | 8.6 |
| I-13 | 2.8 |
| I-14 | 0.2 |
| I-15 | 0.5 |
| I-16 | 0.7 |
| I-17 | 0.4 |
| I-19 | 25 |
| I-20 | 2.5 |
| I-21 | 5.3 |
| I-22 | 0.3 |
| I-24 | 0.8 |

### Example 3 Assay of Drug Transport

### 1. Preparation of Caco-2 Cells

1) 50 μL and 25 mL of cell culture medium were added to each well of the Transwell insert and reservoir, respectively. And then the HTS transwell plates were incubated at 37° C., 5% CO₂ for 1 hour before cell seeding.

2) Caco-2 cells were diluted to 6.86×10⁵ cells/mL with culture medium and 50 μL of cell suspension were dispensed into the filter well of the 96-well HTS Transwell plate. Cells were cultivated for 14-18 days in a cell culture incubator at 37° C., 5% CO₂, 95% relative humidity. Cell culture medium was replaced every other day, beginning no later than 24 hours after initial plating.

### 2. Preparation of Stock Solutions

10 mM stock solutions of test compounds were prepared in DMSO. The stock solutions of positive controls were prepared in DMSO at the concentration of 10 mM. Digoxin and propranolol were used as control compounds in this assay.

### 3. Assessment of Cell Monolayer Integrity

1) Medium was removed from the reservoir and each Transwell insert and replaced with prewarmed fresh culture medium.

2) Transepithelial electrical resistance (TEER) across the monolayer was measured using Millicell Epithelial Volt-Ohm measuring system (Millipore, USA).

3) The Plate was returned to the incubator once the measurement was done.

The TEER value was calculated according to the following equation: TEER measurement (ohms)*Area of membrane (cm²)=TEER value (ohm·cm²) TEER value should be greater than 230 ohm·cm², which indicates the well-qualified Caco-2 monolayer.

### 4. Assay Procedures

1) The Caco-2 plate was removed from the incubator and washed twice with pre-warmed HBSS (10 mM HEPES, pH 7.4), and then incubated at 37° C. for 30 minutes.

2) The stock solutions of control compounds and test compounds were diluted in DMSO to get 1 mM solutions and then diluted with HBSS (10 mM HEPES, pH 7.4) get 5 μM working solutions. The final concentration of DMSO in the incubation system was 0.5%.

3) To determine the rate of drug transport in the apical to basolateral direction. 125 μL of 5 μM working solution of control compound and test compounds were added to the Transwell insert (apical compartment), and transfer 50 μL sample (DO sample) immediately from the apical compartment to a new 96-well plate. Fill the wells in the receiver plate (basolateral compartment) with 235 μL of HBSS (10 mM HEPES, pH 7.4).

4) To determine the rate of drug transport in the basolateral to apical direction. 285 μL of 5 μM working solution of control compound and test compounds were to the receiver plate wells (basolateral compartment), and transfer 50 μL sample (DO sample) immediately from the basolateral compartment to a new 96-well plate. Fill the wells in the Transwell insert (apical compartment) with 75 μL of HBSS (10 mM HEPES, pH 7.4). The assay was performed in duplicate.

5) The plates were incubated at 37° C. for 2 hours.

6) At the end of the incubation, 50 μL samples from donor sides (apical compartment for Ap→Bl flux, and basolateral compartment for Bl→Ap) and receiver sides (basolateral compartment for Ap→Bl flux, and apical compartment for Bl→Ap) were transferred to wells of a new 96-well plate, followed by the addition of 4 volume of cold methanol containing appropriate internal standards (IS). Samples were Vortexed for 5 minutes and then centrifuged at 3,220 g for 40 minutes. An aliquot of 100 μL of the supernatant was mixed with an appropriate volume of ultra-pure water before LC-MS/MS analysis.

7) To determine the Lucifer Yellow leakage after 2 hour transport period, stock solution of Lucifer yellow was prepared in water and diluted with HBSS (10 mM HEPES, pH 7.4) to reach the final concentration of 100 μM. 100 μL of the Lucifer yellow solution was added to each Transwell insert (apical compartment), followed by filling the wells in the receiver plate (basolateral compartment) with 300 μL of HBSS (10 mM HEPES, pH 7.4). The plates were Incubated at 37° C. for 30 mins. 80 μL samples were removed directly from the apical and basolateral wells (using the basolateral access holes) and transferred to wells of new 96 wells plates. The Lucifer Yellow fluorescence (to monitor monolayer integrity) signal was measured in a fluorescence plate reader at 485 nM excitation and 530 nM emission.

### 5. Data Analysis

The apparent permeability coefficient (Pₐₚₚ), in units of centimeter per second, can be calculated for Caco-2 drug transport assays using the following equation:

$${P}_{app} = ( {V}_{A} \times {[ drug ]}_{acceptor} ) / ( Area \times Time \times {[ drug ]}_{initial , donor} )$$

Where V_{A} is the volume (in mL) in the acceptor well, Area is the surface area of the membrane (0.143 cm² for Transwell-96 Well Permeable Supports), and time is the total transport time in seconds.

The efflux ratio will be determined using the following equation:

$$Efflux Ratio = {P}_{app ( B - A )} / {P}_{app ( A - B )}$$

Where P_{app (B-A)} indicates the apparent permeability coefficient in basolateral to apical direction, and P_{app (A-B)} indicates the apparent permeability coefficient in apical to basolateral direction.

The recovery can be determined using the following equation:

$$Recovery \% = ( {V}_{A} \times {[ drug ]}_{acceptor} + {V}_{D} \times {[ drug ]}_{donor} ) / ( {V}_{D} \times {[ drug ]}_{initial , donor} )$$

Where V_{A} is the volume (in mL) in the acceptor well (0.235 mL for Ap→Bl flux, and 0.075 mL for Bl→Ap), V_{D} is the volume (in mL) in the donor well (0.075 mL for Ap→BI flux, and 0.235 mL for Bl→Ap)

The leakage of Lucifer Yellow, in unit of percentage (%), can be calculated using the following equation:

$$\% LY leakage = 100 \times {[ LY ]}_{acceptor} / ( {[ LY ]}_{donor} + {[ LY ]}_{acceptor} )$$

LY leakage of <1% is acceptable to indicate the well-qualified Caco-2 monolayer.

The P_{app (B-A)}, P_{app (A-B)} and Efflux ratio are summarized in Table 3.

TABLE 3

| | P_{app (B-A)} | P_{app (A-B)} | |
|---|---|---|---|
| Compound | (10⁻⁶, cm/s) | (10⁻⁶, cm/s) | Efflux Ratio |
| Digoxin | 0.30 | 16.98 | 56.52 |
| Propranolol | 26.55 | 13.63 | 0.51 |
| I-14 | 12.98 | 31.49 | 2.43 |
| I-15 | 24.78 | 25.20 | 1.02 |
| I-16 | 22.43 | 20.84 | 0.93 |
| I-17 | 25.26 | 19.35 | 0.77 |
| I-22 | 12.74 | 32.13 | 2.52 |
| I-24 | 5.28 | 18.45 | 3.49 |

### Example 4 Assay of Intrinsic Clearance

1. The master solution was prepared according to Table 4.

TABLE 4

| Reagent | Stock Concentration | Volume | Final Concentration |
|---|---|---|---|
| Phosphate buffer | 200 mM | 200 μL | 100 mM |
| Ultra-pure H₂O | — | 106 μL | — |
| MgCl₂ solution | 50 mM | 40 μL | 5 mM |

2. Three separated experiments were performed as follows. a) With NADPH: 10 μL of 20 mg/mL liver microsomes and 40 μL of 10 mM NADPH were added to the incubations. The final concentrations of microsomes and NADPH were 0.5 mg/mL and 1 mM, respectively. b) Without NADPH: 10 μL of 20 mg/mL liver microsomes and 40 μL of ultra-pure H₂O were added to the incubations. The final concentration of microsomes was 0.5 mg/mL. c) Heat-inactivated microsomes without NADPH: 10 μL of 20 mg/mL heat-inactivated liver microsomes and 40 μL of ultra-pure H₂O were added to the incubations. The final concentration of microsomes was 0.5 mg/mL.

3. The reaction was started with the addition of 4 μL of 200 μM test compound solution or control compound solution at the final concentration of 2 μM and carried out at 37° C.

4. Aliquots of 50 μL were taken from the reaction solution at 0, 15, 30, 45 and 60 min. The reaction was stopped by the addition of 4 volumes of cold acetonitrile with IS (100 nM alprazolam, 200 nM labetalol, 200 nM caffeine and 2 μM ketoprofen). Samples were centrifuged at 3, 220 g for 40 minutes. That is to say, the centrifugal force is 3220 times the acceleration of gravity. Aliquot of 100 μL of the supernatant was mixed with 100 μL of ultra-pure H₂O and then used for LC-MS/MS analysis.

5. Data Analysis

All calculations were carried out using Microsoft Excel.

Peak areas were determined from extracted ion chromatograms. The slope value, k, was determined by linear regression of the natural logarithm of the remaining percentage of the parent drug vs. incubation time curve.

The in vitro half-life (in vitro t_{1/2}) was determined from the slope value:

in vitro t_{1/2}=−(0.693/k)

Conversion of the in vitro t_{1/2} (min) into the in vitro intrinsic clearance (in vitro CLᵢₙₜ, in μL/min/mg protein) was done using the following equation (mean of duplicate determinations):

$$in vitro {CL}_{i n t} = ( 0.693 * volume of incubation ( µl ) ) / ( in vitro {t}_{1 / 2} * amount of proteins ( mg ) )$$

Conversion of the in vitro t_{1/2} (min) into the scale-up unbound intrinsic clearance (Scale-up CLᵢₙₜ, in mL/min/kg) was done using the following equation (mean of duplicate determinations):

The Scaling Factors for Intrinsic Clearance Prediction in Liver Microsomes are summarized in Table 5.

TABLE 5

| | Liver Weight | Microsomal | Liver blood | |
|---|---|---|---|---|
| | (g liver/kg | Concentration | flow (Q, | Scaling |
| Species | body weight)^{a} | (mg/g liver)^{b} | mL/min/kg)^{a} | Factor |
| Human | 25.7 | 48.8 | 20.7 | 1254.2 |
| Monkey | 30.0 | 50.0 | 43.6 | 1500.0 |
| Dog | 32.0 | 77.9 | 30.9 | 2492.8 |
| Rat | 40.0 | 44.8 | 55.2 | 1792.0 |
| Mouse | 88.0 | 50.0 | 90.0 | 4400.0 |

^{a}Iwatsubo et al, Davies and Morris, 1993, 10 (7) pp 1093-1095.
^{b}Barter et al, 2007, Curr Drug Metab, 8(1), pp 33-45; Iwatsubo et al, 1997, JPET, 283 pp 462-469.

The in vitro CLᵢₙₜ is summarized in Table 6.

TABLE 6

| | | | in vitro CLᵢₙₜ |
|---|---|---|---|
| | | in vitro t_{1/2} | (μL/min/mg |
| Compound | Species | (min) | protein) |
| I-14 | Human | 1493.02 | 0.93 |
| | Mouse | 234.96 | 5.90 |
| I-15 | Mouse | 35.72 | 38.80 |
| I-16 | Human | 111.01 | 12.49 |
| | Mouse | 20.74 | 66.84 |
| I-17 | Human | 426.61 | 3.25 |
| | Mouse | 72.56 | 19.10 |
| I-22 | Human | 525.37 | 2.64 |
| | Mouse | 73.16 | 18.94 |
| I-24 | Human | 185.62 | 7.47 |
| | Mouse | 56.10 | 24.70 |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A compound having the structure of formula (I), 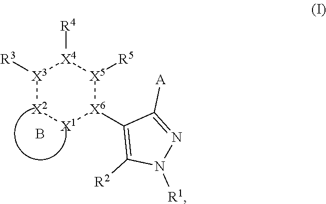
or a pharmaceutically acceptable salt, or prodrug thereof, or a solvate or hydrate of any of the forgoing, or a composition comprising any of the forgoing and optionally a pharmaceutically acceptable carrier,
wherein,
each A and B is independently selected from the group consisting of optionally substituted C₆-C₁₄ aryl, and optionally substituted C₂-C₉ heteroaryl;
each dashed line ( [symbol] ) represents a single or double bond, each X¹, X², X³, X⁴, X⁵ and X⁶ is independently selected from the group consisting of C, N, and optionally substituted CH;
R¹ is selected from the group consisting of hydrogen, protium, deuterium, tritium, halogen, cyano, nitro, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl;
R² is selected from the group consisting of hydrogen, protium, deuterium, tritium, halogen, cyano, nitro, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl,
or R¹ and R² combined with the atoms to which they are attached form an optionally substituted ring;
each R³, R⁴ and R⁵ is independently absent or is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl,
or R³ and R⁴ combined with the atoms to which they are attached form an optionally substituted ring,
or R³ and R⁵ combined with the atoms to which they are attached form an optionally substituted ring,
or R⁴ and R⁵ combined with the atoms to which they are attached form an optionally substituted ring.

2. The compound of claim 1, wherein, said B is optionally substituted C₂-C₉ heteroaryl; or
said B is selected from the group consisting of optionally substituted pyrazole, optionally substituted imidazole, optionally substituted thiophene, optionally substituted pyrrole, and optionally substituted triazole; or said B is optionally substituted imidazole; or
said B is optionally substituted imidazole.

3. (canceled)

4. (canceled)

5. The compound of claim 1, wherein, said B is substituted with one or more R⁶, each R⁶ is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl.

6. The compound of claim 5, wherein, each R⁶ is independently selected from the group consisting of hydrogen, halogen, ═O, optionally substituted (C₁-C₆)alkyl, optionally substituted (C₁-C₆)acyl, and optionally substituted amino; or
each R⁶ is independently selected from the group consisting of optionally substituted methyl, optionally substituted ethyl and optionally substituted isopropyl.

7. (canceled)

8. The compound of claim 5, wherein, said R⁶ is substituted with one or more R⁷, each R⁷ is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl; or
said R⁷ is independently selected from the group consisting of hydrogen, optionally substituted (C₁-C₆)alkyl, optionally substituted (C₁-C₆)acyl, optionally substituted amino, and optionally substituted hydroxy.

9. (canceled)

10. The compound of claim 8, wherein, said R⁷ is substituted with one or more R⁸, each R⁸ is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl or
said R⁸ is independently selected from the group consisting of hydrogen, optionally substituted (C₁-C₆)alkyl, optionally substituted (C₃-C₁₀)carbocycle, and optionally substituted (C₁-C₆)acyl; or
said R⁸ is independently selected from the group consisting of optionally substituted methyl and optionally substituted cyclopropyl.

11. (canceled)

12. (canceled)

13. The compound of claim 10, wherein, said R⁸ is substituted with one or more R⁹, each R⁹ is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl; or
said R⁹ is independently selected from the group consisting of hydrogen, halogen and optionally substituted (C₁-C₆)alkyl.

14. (canceled)

15. The compound of claim 1, wherein, each X¹ and X² is independently selected from the group consisting of C and N; or
said X¹ is C and said X² is N.

16. (canceled)

17. The compound of claim 1, wherein the compound has the structure selected from the group consisting of

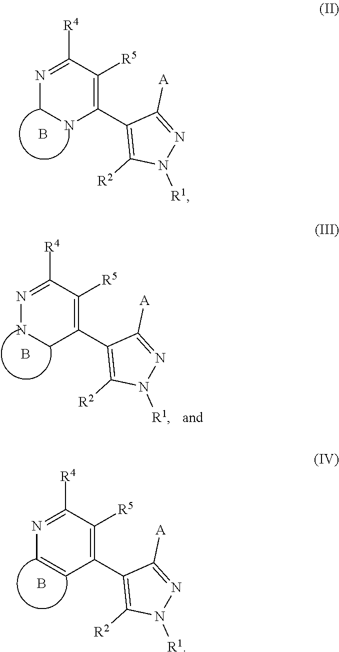

18. The compound of claim 1, wherein, each R⁴ and R⁵ is independently selected from the group consisting of hydrogen, protium, deuterium, tritium, halogen, cyano, nitro, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl
each R⁴ and R⁵ is independently selected from the group consisting of hydrogen and halogen; or
said R⁴ is hydrogen and said R⁵ is hydrogen.

19. (canceled)

20. (canceled)

21. The compound of claim 1, wherein, said R¹ is optionally substituted (C₁-C₆)alkyl; or said R¹ is optionally substituted methyl.

22. (canceled)

23. (canceled)

24. The compound of claim 1, wherein, R¹ and R² combined with the atoms to which they are attached form an optionally substituted ring C, said ring C is selected from the group consisting of optionally substituted (C₃-C₁₀) carbocycle, optionally substituted (C₂-C₉) heterocycle, optionally substituted (C₆-C₁₀) aryl, and optionally substituted (C₁-C₉) heteroaryl; or
said ring C is optionally substituted (C_{Z}-C_{g}) heterocycle; or
said ring C is optionally substituted piperazine.

25. (canceled)

26. (canceled)

27. The compound of claim 24, wherein, said ring C is substituted with one or more R¹⁰, each R¹⁰ is independently absent or is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl; or
said R¹⁰ is independently selected from the group consisting of optionally substituted (C₁-C₆)acyl and optionally substituted (C₁-C₆)alkyl.

28. (canceled)

29. The compound of claim 1, wherein, said A is optionally substituted C₆-C₁₄ aryl; or said A is optionally substituted phenyl.

30. (canceled)

31. The compound of claim 1, wherein, said A is substituted with one or more R¹¹, each R¹¹ is independently absent or is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆)acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀)aryl, and optionally substituted (C₁-C₉)heteroaryl; or
said R¹¹ is halogen; or
said R¹¹ is F.

32. (canceled)

33. (canceled)

34. The compound of claim 1, wherein, said compound is selected from the group consisting of

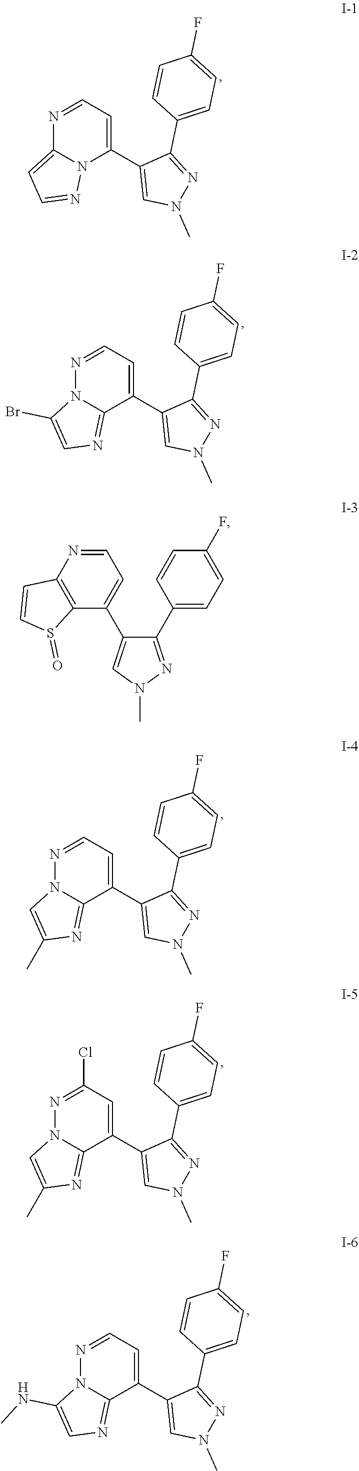

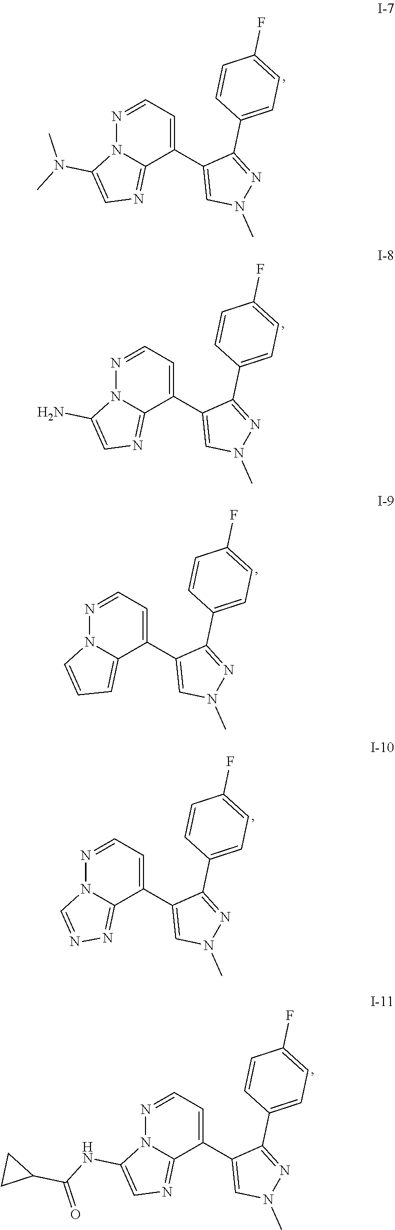

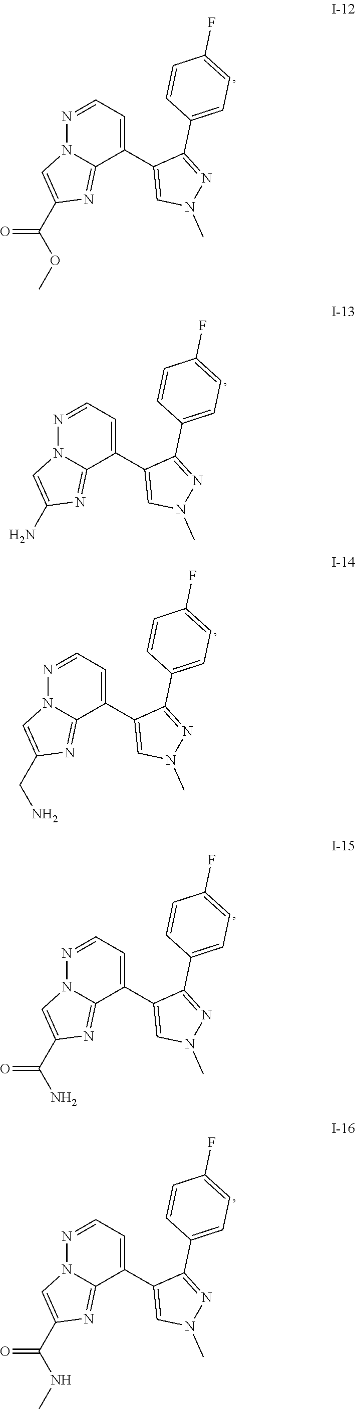

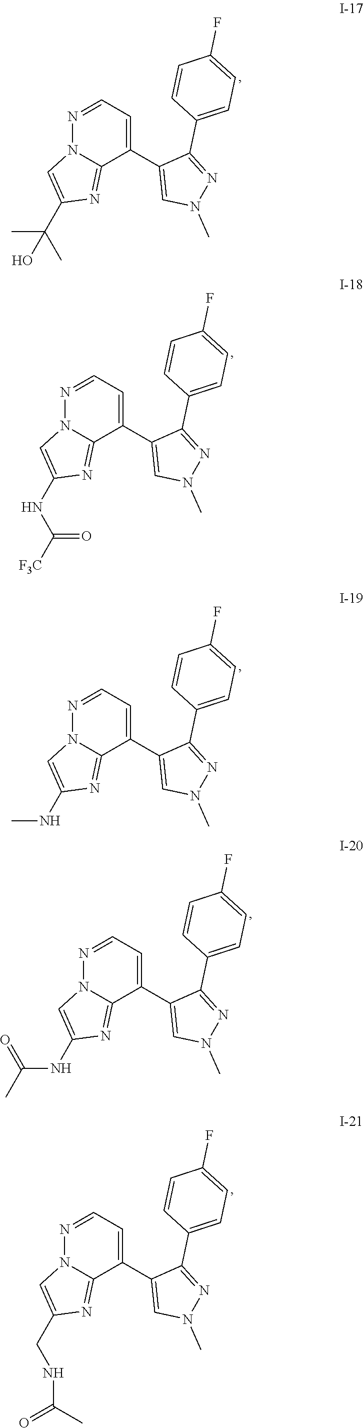

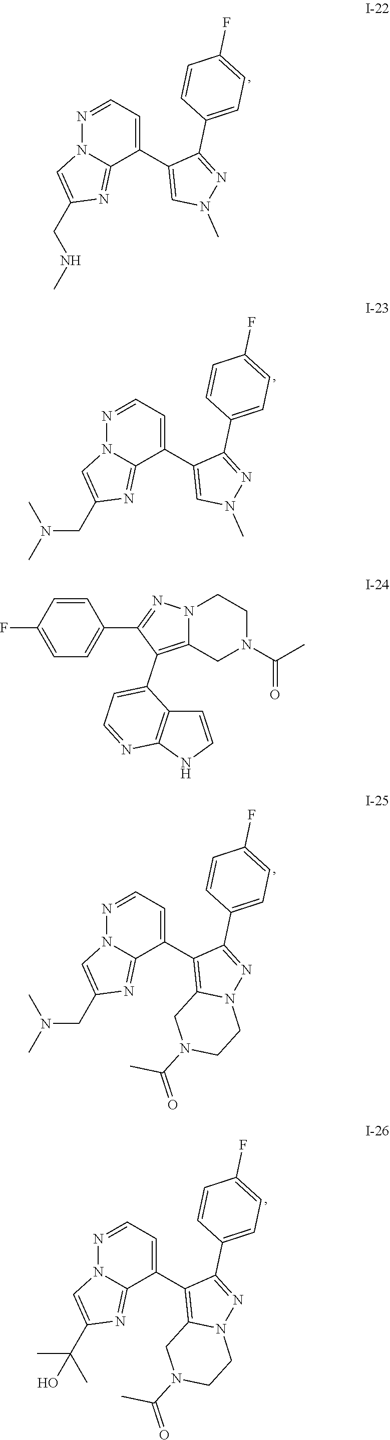

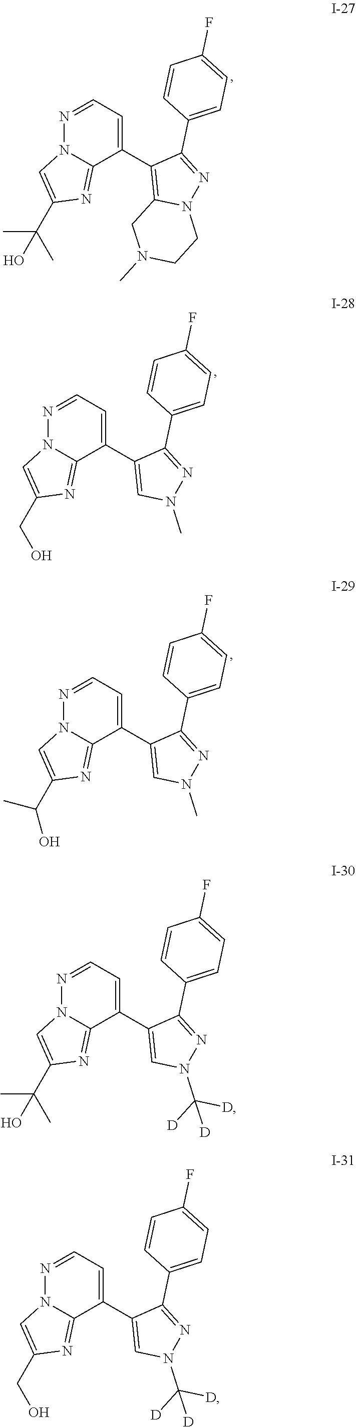

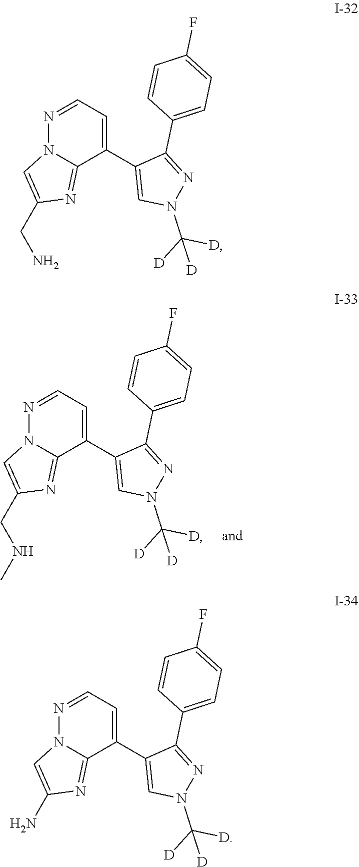

35. (canceled)

36. A method for inhibiting casein kinase (CK) activity, said method comprising administering to a subject in need thereof an effective amount of the compound of claim 1, or a pharmaceutically acceptable salt, prodrug, or metabolite thereof, or a solvate or hydrate of any of the foregoing.

37. The method of claim 36, wherein said casein kinase (CK) is selected from the group consisting of casein kinase I alpha (CK1α), casein kinase I delta (CK1δ) and casein kinase I epsilon (CK1ε) or said method is selected from the group consisting of an in vitro method, an ex vivo method, and an in vivo method.

38. (canceled)

39. A method for preventing and/or treating a disease or disorder, said method comprising administering to a subject in need thereof an effective amount of the compound of claim 1, or a pharmaceutically acceptable salt, prodrug, or metabolite thereof, or a solvate or hydrate of any of the foregoing.

40. The method of claim 39, wherein said disease or disorder is selected from the group consisting of neurological disease and psychiatric disease; or said disease or disorder is selected from the group consisting of mood disorder, sleep disorder, and circadian disorder: or said disease or disorder is selected from the group consisting of depressive disorder and bipolar disorder.

41. (canceled)

42. (canceled)
